(19) United States
(12) Patent Application Publication
(10) Pub. No.: US 2026/0199505 A1
(43) Pub. Date: Jul. 16, 2026

(54) COMBINATION THERAPY COMPRISING BISPECIFIC ANTIBODIES COMPRISING AN NRP1 BINDING DOMAIN

(71) Applicant: Pinetree Therapeutics, Inc., Cambridge, MA (US)

(72) Inventor: Hojuhn SONG, Andover, MA (US)

(21) Appl. No.: 19/134,151

(22) PCT Filed: Aug. 28, 2023
(86) PCT No.: PCT/US2023/031268

§ 371 Date: May 29, 2025

Related U.S. Application Data
(60) Provisional application No. 63/423,456, filed on Nov. 7, 2022.

(51) Int. Cl. A61K 47/68; A61K 31/4545; A61K 31/506; A61K 31/519; A61P 35/00

(52) U.S. Cl. CPC A61K 47/6849; A61K 31/4545; A61K 31/506; A61K 31/519; A61P 35/00

(57) ABSTRACT

A method of enhancing the therapeutic effects of a therapeutic agent in a subject having a cancer that is resistant to the therapeutic agent includes administering to the subject (i) a therapeutic agent and (ii) a target protein degrader that synergistically increases the therapeutic activity of the therapeutic agent by reversing or reducing the cancer resistance to the therapeutic agent; and thereby enhancing the therapeutic effects of the therapeutic agent in the subject.

### RELATED APPLICATIONS

This application claims priority to U.S. Provisional Application No. 63/423,456, filed Nov. 7, 2022, the entire contents of which is hereby incorporated by reference.

### BACKGROUND

Targeted therapy is well advanced and initially beneficial to patients with certain oncogenic mutations. However, despite the initial clinical responses to therapeutic drugs, long-term efficacy is not currently possible because acquired drug resistance to targeted therapies hampers the effectiveness of these therapies. Effective treatments are needed.

### SUMMARY

The present disclosure relates to cancer therapies, as well as preventing or reducing drug resistance in cancer. In particular, the present disclosure relates to combination therapies using a targeted protein degrader and one or more therapeutic agents. The present disclosure relates to methods of enhancing the therapeutic effects of a therapeutic agent in a subject having a cancer that is resistant to the therapeutic agent, wherein the therapeutic agent is administered in combination with the targeted protein degrader.

Accordingly, in one aspect, the disclosure pertains to a method of enhancing effects of a therapeutic agent in a subject having a cancer that is resistant or refractory to the therapeutic agent, the method comprising:
administering to the subject (i) the therapeutic agent; and (ii) a target protein degrader, such that the effects of the therapeutic agent are enhanced as compared to administering the therapeutic agent alone;
wherein the target protein degrader comprises a bispecific binding molecule comprising:
   (a) a target protein binding domain that specifically binds to a target protein on the subject's cancer cells; and
   (b) a neuropilin-1 (NRP1) binding domain that binds to NRP1 comprising an antibody or NRP-1-binding fragment thereof.

In an embodiment, the target protein is a receptor tyrosine kinase (RTK). In other embodiments, the receptor tyrosine kinase is selected from epidermal growth factor receptors (EGFRs), platelet-derived growth factor receptors (PDGFRs), fibroblast growth factor receptors (FGFRs), Met receptors tyrosine kinase (METs), and vascular endothelial growth factors (VEGFRs). In an embodiment, the receptor tyrosine kinase is EGFR. In an embodiment, the receptor tyrosine kinase is cMET. In an embodiment, the receptor tyrosine kinase is HER2. In an embodiment, the receptor tyrosine kinase is IGF1R.

In an embodiment, the target cell is a cancer cell, such as a cancer cell selected from the group consisting of lung cancer, breast cancer, colon and rectum cancer, head and neck cancer, esophagogastric cancer, liver cancer, glioblastoma, prostate cancer, cervical cancer, ovarian cancer, bladder cancer, kidney cancer, and pancreatic cancer. In an embodiment, the cancer cell is a non-small cell lung cancer (NSCLC) cell.

In an embodiment, the target protein binding domain and the NRP1 binding domain are each independently selected from the group consisting of IgG, half antibodies, single-domain antibodies, nanobodies, Fabs, monospecific Fab2, Fc, scFv, minibodies, IgNAR, V-NAR, hcIgG, VHH domain, camelid antibodies, and peptibodies.

In an embodiment, the NRP1 binding domain comprises:
(i) an antibody heavy chain variable (VH) domain comprising CDR1, CDR2 and CDR3 regions (HCDR1, HCDR2 and HCDR3, respectively), wherein HCDR1 consists of the sequence shown in SEQ ID NO: 79, HCDR2 consists of the sequence shown in SEQ ID NO: 80, and HCDR3 consists of the sequence shown in any one of SEQ ID NOs: 81-84; and
(ii) an antibody light chain variable (VL) domain comprising CDR1, CDR2 and CDR3 regions (LCDR1, LCDR2 and LCDR3, respectively), wherein LCDR1 consists of the sequence shown in any one of SEQ ID NOs: 85-87, LCDR2 consists of the sequence shown in SEQ ID NO: 88, and LCDR3 consists of the sequence shown in SEQ ID NO: 89.

In an embodiment,
(i) HCDR1 consists of the sequence shown in SEQ ID NO: 79, HCDR2 consists of the sequence shown in SEQ ID NO: 80, and HCDR3 consists of the sequence shown in any one of SEQ ID NO: 84; and
(ii) LCDR1 consists of the sequence shown in SEQ ID NO: 85, LCDR2 consists of the sequence shown in SEQ ID NO: 88, and LCDR3 consists of the sequence shown in SEQ ID NO: 89.

In an embodiment, the enhanced effects comprise increased tumor growth inhibition. In an embodiment, the enhanced effects comprise increased median survival time.

In an embodiment, the therapeutic agent targets the EGFR pathway, non-limiting examples of which are disclosed herein. In an embodiment, the therapeutic agent targeting the EGFR pathway is Osimertinib.

In an embodiment, the therapeutic agent targets the cMET pathway, non-limiting examples of which are disclosed herein. In an embodiment, the therapeutic agent targeting the cMET pathway is Crizotinib.

In an embodiment, the therapeutic agent targets a KRAS protein (e.g., a mutant KRAS protein), non-limiting examples of which are disclosed herein. In an embodiment, the therapeutic agent targeting a KRAS protein is sotorasib.

In other embodiments, the therapeutic agent targets, for example, a protein selected from the group consisting of HER2, IGF1R, ALK, Braf, VEGF, and PDGF.

In an embodiment, the method further comprises administering to the subject a second therapeutic agent, wherein the effects of the second therapeutic agent are enhanced by the protein target degrader as compared to administering the second therapeutic agent alone. In an embodiment, the therapeutic agent targets EGFR and the second therapeutic agent targets cMET, for example in an embodiment the therapeutic agent is Osimertinib and the second therapeutic agent is sotorasib.

In another aspect, the disclosure pertains to a method of enhancing effects of a receptor tyrosine kinase (RTK) inhibitor in a subject having a cancer that is resistant or refractory to the RTK inhibitor, the method comprising:
administering to the subject (i) the RTK inhibitor; and (ii) a target protein degrader, such that the effects of the RTK inhibitor are enhanced as compared to administering the RTK inhibitor alone;
wherein the target protein degrader comprises a bispecific binding molecule comprising:
   (a) a target protein binding domain that specifically binds to an RTK on the subject's cancer cells; and
   (b) a neuropilin-1 (NRP1) binding domain that binds to NRP1 comprising an antibody or NRP-1-binding fragment thereof.

In another aspect, the disclosure pertains to a method of enhancing effects of an epidermal growth factor receptor (EGFR) inhibitor in a subject having a cancer that is resistant or refractory to the EGFR inhibitor, the method comprising:
administering to the subject (i) the EGFR inhibitor; and (ii) a target protein degrader, such that the effects of the EGFR inhibitor are enhanced as compared to administering the EGFR inhibitor alone;
wherein the target protein degrader comprises a bispecific binding molecule comprising:
   (a) a target protein binding domain that specifically binds to EGFR on the subject's cancer cells; and
   (b) a neuropilin-1 (NRP1) binding domain that binds to NRP1 comprising an antibody or NRP-1-binding fragment thereof.

The present disclosure provides relates to methods of enhancing the therapeutic effects of a therapeutic agent in a subject having a cancer that is resistant to the therapeutic agent: (i) administering to the subject a target protein degrader and a therapeutic agent that synergistically increases the therapeutic activity of the therapeutic agent by reversing or reducing the cancer resistance to the cancer therapeutic agent in the subject.

In some embodiments, the cancer is resistant to a chemotherapeutic agent.

The present disclosure provides methods of sensitizing a therapeutic agent-resistant cancer in a subject to the therapeutic agent. The methods include administering to the subject a target protein degrader that degrades the target protein and thereby inducing tumor regression in a subject.

In some embodiments, the methods include a pharmaceutical composition comprising: a bispecific binding molecule that degrades a target protein. In some embodiments, the pharmaceutical composition comprises the therapeutically effective amount of the bispecific binding molecule and a pharmaceutically acceptable carrier or diluent.

In some embodiments, the medicaments provide a kit, and the kit also comprises a package insert comprising instructions for using a pharmaceutical composition comprising the therapeutically effective amount of the bispecific binding molecule and a pharmaceutically acceptable carrier or diluent.

In some embodiments, the bispecific binding molecule comprises an EGFR degrader. In some embodiments, the bispecific binding molecule comprises a first polypeptide having a sequence of SEQ ID NO: 11 and a second polypeptide having a sequence of SEQ ID NO:12.

In some embodiments, the cancer comprises lung cancer, breast cancer, colon and rectum cancer, head and neck cancer, esophagogastric cancer, liver cancer, glioblastoma, prostate cancer, cervical cancer, ovarian cancer, bladder cancer, kidney cancer, and pancreatic cancer. In some embodiments, the cancer is non-small cell lung cancer (NSCLC).

In some embodiments, the therapeutic agent comprises the receptor tyrosine kinase comprises epidermal growth factor receptors (EGFRs) inhibitors, ERBB inhibitors, c-MET inhibitors, fibroblast growth factor receptors (FGFRs) and platelet-derived growth factor receptors (PDGFRs) inhibitors, CSF1R inhibitors, cKIT inhibitors, FTL3 inhibitors, and receptors vascular endothelial growth factors (VEGFRs) inhibitors, TGFIβ1R and TGFβ2R inhibitors, Integrin inhibitors, and IGF1R and IR inhibitors.

In some embodiments, the EGFR inhibitor is selected from the group consisting of Lazertinib, Osimertinib (AZD9291), WZ4002, Cyasterone, Erlotinib (OSI-774) HCl, efitinib (ZD1839), Lapatinib (GW-572016) Ditosylate, Afatinib (BIBW2992), Saracatinib (AZD0530), Vandetanib (ZD6474), Neratinib (HKI-272), Canertinib (CI-1033), Lapatinib (GW-572016), AG-490 (Tyrphostin B42), CP-724714, Dacomitinib (PF-00299804), Sapitinib (AZD8931), CUDC-101, AG-1478 (Tyrphostin AG-1478), PD153035 HCl, Pelitinib (EKB-569), AC480 (BMS-599626), AEE788 (NVP-AEE788), AP26113-analog (ALK-IN-1), OSI-420, WZ3146, HER2-Inhibitor-1, WZ8040, Allitinib tosylate, Rociletinib (CO-1686), Genistein (NPI 031L), Varlitinib, TQB33804 (EGFR-IN-7), Icotinib (BPI-2009H), TAK-285, Daphnetin, Tyrphostin 9, AG-18, AG 555, AZ5104, CL-387785 (EKI-785), Tyrphostin AG-258, AG-556, Tucatinib, Erlotinib (OSI-774), Gefitinib-based PROTAC 3, Zorifertinib (AZD 3759), ErbB2, AV-412 free base, AST-1306, JND3229, BI-4020, Theliatinib (HMPL-309), BDTX-189, Lifirafenib (BGB-283), Pyrotinib (SHR-1258), O-Demethyl-Gefitinib, Epertinib hydrochloride, SU5214, Avitinib (ACO0010), AG 494, and Poziotinib (HM781-36B). In certain embodiments, the EGFR inhibitor is N-(2-{2-dimethylaminoethyl-methylamino}-4-methoxy-5-{[4-(1-methylindol-3-yl)pyrindin-2-yl]amino}phenyl)prop-2-enamide mesylate salt (Osimertinib).

In some embodiments, the therapeutic agent comprises ERBB inhibitors. In some embodiments, the ERBB inhibitor is selected from the group consisting of Tucatinib, HER2-inhibitor-1, Afatinib (BIBW2992), Neratinib (HKI-272), CP-724714, Mubritinib (TAK 165), AC480 (BMS-599626), AEE778, TAK-285, Tyrphostin AG 879, Tyrphostin AG-528, SU5204, Poziotinib (HM781-36B), TAS0728, BDTX-189, Pyrotinib, and Epertinib Hydrochloride.

In some embodiments, the therapeutic agent comprises cMET inhibitors. In some embodiments, the cMET inhibitor is selected from the group consisting of Crizotinib, Cabozzantinib, Foretinib, PHA-665752, SU11274, SGX-523, BMS-777607, Tivantinib, JNJ-38877605, PF-04217903, Amuvatinib (MP-470), MGCD-265 analog, Capmatinib, BMS-754807, BMS-794833, AMG-208, MK-2461, Golvatinib, AMG-458, NVP-BVU972, AMG 337, Merestinib, JNJ-38877618, Crizotinib hydrochlorode, Ningetinib, AMG-1, UNC2025, Pamufetinib, Altiratinib, NPS-1304, and Savolitinib.

In some embodiments, the therapeutic agent comprises PDGFR inhibitors and FGFR inhibitors.

In some embodiments, the PDGFR inhibitor and FGFR inhibitor is selected from the group consisting of Ponatinib (AP24534), Infigratinib (BGJ398), Nintedanib (BIBF 1120), Pazopanib HCl (GW786034 HCl), Pazopanib, AZD4547, Tyrphostin AG 1296, SSR128129E, LY2874455, Derazantinib (ARQ-087), SU5402, ODM-203, Pemigatinib (INCB054828), Lucitanib (E3810) hydrochloride, Ferulic Acid, Masitinib mesylate, Fisogatinib (BLU-554), PRN1371, ON123300, FIIN-3, Roblitinib (FGF401), Futibatinib (TAS-120), FIIN-2, Zoligratinib (Debio-1347), Nintedanib Ethanesulfonate Salt, BLU9931, Sulfatinib, and combination thereof.

In some embodiments, the therapeutic agent comprises CSF1R inhibitors. In some embodiments, the CSF1R inhibitor is selected from the group consisting of CSF1R-IN-1, Ki20227, Pazopanib, Elzovantinib (TPX-0022), ARRY-382, PRN1371, ENMD-2076, PF-477736, Sulfatinib, and combination thereof.

In some embodiments, the therapeutic agent comprises cKIT inhibitors. In some embodiments, the cKIT inhibitor is selected from the group consisting of Dasatinib (BMS-354825), Sorafenib (BAY 43-9006) tosylate, Imatinib (ST1571) Mesylate, Sunitinib (SU11248) malate, Ponatinib (AP24534), Axitinib (AG 013736), Imatinib (ST1571), Nintedanib (BIBF 1120), Regorafenib (BAY 73-4506), Pazopanib HCl (GW786034 HCl), Linifanib (ABT-869), Crenolanib (CP-868596), Masitinib (AB1010), Amuvatinib (MP-470), Orantinib (SU6668), CP-673451, Telatinib, PP121, Pazopanib, Tyrphostin AG 1296, Tyrphostin 9, SU14813, Regorafenib Hydrochloride, Tyrphostin AG1433, Sunitinib (SU11248), Ripretinib (DCC-2618), Masitinib mesylate, AZD3229, ON123300, Avapritinib (BLU-285), Seralutinib (GB002), AZD2932, JNJ-10198409, Nintedanib Ethanesulfonate Salt, Flumatinib (HH-GV-678), Regorafenib (BAY-734506) Monohydrate, and combination thereof.

In some embodiments, the therapeutic agent comprises FLT3 inhibitors. In some embodiments, the FLT3 inhibitor is selected from the group consisting of Pacritinib (SB1518), TCS 359, Linifanib (ABT-869), Zotiraciclib, Cediranib (AZD2171), Dovitinib (TK1258) Lactate, UNC2025 HCl, SU5614, FLT3-IN-2, FLT3-IN-4, FF-10101, Merestinib (LY2801653), Emavusertib (CA-4948), Tandutinib (MLN518), R406 (free base), 5′-Fluoroindirubinoxime, Tozasertib, Quizartinib (AC220), R406, AST-487 (NVP-AST487), Sorafenib (BAY 43-9006) tosylate, BMS-794833, Sorafenib (BAY 43-9006), 4SC-203, Dovitinib (TKI-258), Isoguanosine, TAK-659, ATH686, SGI-1776 free base, MK-2461, Fostamatinib (R788) disodium, MRX-2843, Brigatinib (AP26113), GW2580, Rebastinib (DCC-2036), BMS-754807, UNC2025, Fedratinib (TG101348), FLT3-IN-3, G-749, Crenolanib (CP-868596), BPR1K871, PHA-680632, Entospletinib (GS-9973), HPK1-IN-2, SP600125, SKLB4771 (FLT3-IN-1), KW-2449, Gilteritinib (ASP2215), CCT241736, PRT062607 (P505-15) HCl, ENMD-2076, FN-1501, Ceritinib (LDK378), Silmitasertib (CX-4945), AZD2932, Fostamatinib (R788), Tivozanib (AV-951), PF-477736, BPR1J-097, Pexidartinib (PLX3397), Go6976, HM43239, OSI-930, TG101209, PLX5622, Amuvatinib (MP-470), GNF-2, Midostaurin (PKC412), AMG 925, and ENMD-2076 L-(+)-Tartaric acid.

In some embodiments, the therapeutic agent comprises VEGFR1, VEGFR2, VEGFR3, and VEGFR4 inhibitors. In some embodiments, the VEGFR1, VEGFR2, VEGFR3, or VEGFR4 inhibitor is selected from the group consisting of Sorafenib (BAY 43-9006) tosylate, Sunitinib (SU11248) malate, Lenalidomide (CC-5013), Cabozantinib (BMS-907351), Ponatinib (AP24534), Axitinib (AG 013736), Foretinib (GSK1363089), Vandetanib (ZD6474), Nintedanib (BIBF 1120), Regorafenib (BAY 73-4506), Pazopanib HCl (GW786034 HCl), Cediranib (AZD2171), PD173074, Dovitinib (TKI-258), Linifanib (ABT-869), Vatalanib (PTK787) 2HC1, RAF265 (CHIR-265), Tivozanib (AV-951), Motesanib Diphosphate (AMG-706), Lenvatinib (E7080), Brivanib (BMS-540215), MGCD-265 analog, AEE788 (NVP-AEE788), ENMD-2076, OSI-930, CYC116, Ki8751, Telatinib, PP121, Pazopanib, KRN 633, SAR131675, BMS-794833, Apatinib (YN968D1) mesylate, Sorafenib (BAY 43-9006), Cabozantinib malate, Brivanib Alaninate (BMS-582664), Golvatinib (E7050), Semaxanib (SU5416), ZM 306416, ZM 323881 HCl, ENMD-2076 L-(+)-Tartaric acid, LY2874455, BAW2881 (NVP-BAW2881), WHI-P180, SU14813, ZD-4190, SU1498, SU5402, PDGFR inhibitor 1, Ki20227, Dovitinib (TK1258) Lactate, Toceranib phosphate, Cediranib Maleate, Apatinib, BFH772, Lenvatinib (E7080) Mesylate, SU5614, Regorafenib Hydrochloride, SU5204, SU5208, Fruquintinib (HMPL-013), hVEGF-IN-1, ODM-203, Erdafitinib (JNJ-42756493), Tyrphostin AG1433, MAZ51, SKLB 610, Sunitinib (SU11248), 4SC-203, Sitravatinib (MGCD516), R1530, Donafenib (Sorafenib D3), Emvododstat (PTC299), AG-13958, SKLB1002, Motesanib (AMG-706), 4,4′-Bis(4-aminophenoxy)biphenyl, Lucitanib (E3810) hydrochloride, Oglufanide, Chiauranib, Ningetinib, X-82 (Vorolanib), Pamufetinib (TAS-115), Cassia seed Extract, CS-2660 (JNJ-38158471), WAY-340935, (20R)-Protopanaxadiol, Semen litchi Extract, Altiratinib, Vitamin E, SU5408, AZD2932, Anlotinib (AL3818) dihydrochloride, Nintedanib Ethanesulfonate Salt, Chebulinic acid, SU5205, SU5214, Regorafenib (BAY-734506) Monohydrate, Taxifolin (Dihydroquercetin), Sulfatinib, XL092, and combination thereof.

In some embodiments, the therapeutic agent comprises TGFP1R and TGFβ2R inhibitors. In some embodiments, the TGFP1R or TGFP2R inhibitor is selected from the group consisting of SD-208, GW788388, A-83-01, Disitertide (P144), SR1-011381, TP0427736 HCl, LY2109761, Ophiopogonin D, SB505124, SIS3 HCl, BIBF-0775, LY 3200882, LSKL, Inhibitor of Thrombospondin (TSP-1), Galunisertib (LY2157299), Ginsenoside Rh4, LDN-193189, A77-01, LDN-193189 2HC1, Vactosertib (TEW-7197), Halofuginone hydrobromide, Halofuginone, Sulfasalazine (NSC 667219), BMS-986260, XAV-939, LY364947, Oxymatrine, Pirfenidone (S-7701), Hypaconitine, SB525334, ITD-1, Gamabufotalin, TA-02, PD 169316, and combination thereof.

In some embodiments, the therapeutic agent comprises Integrin inhibitors. In some embodiments, the Integrin inhibitor is selected from the group consisting of Cilengitide trifluoroacetate, RGD (Arg-Gly-Asp) Peptides, A-205804, SB273005, Cilengitide, RGD peptide (GRGDNP), OSU-T315, ILK-IN-3, Cyclo (-RGDfK), A286982, Cyclo(RGDyK), A286982, and combination thereof.

In some embodiments, the therapeutic agent comprises IGF1R inhibitors and IR inhibitors. In some embodiments, the IGF1R inhibitor or IR inhibitor is selected from the group consisting of Luminespib (NVP-AUY922), Linsitinib (OSI-906), NVP-AEW541, GSK1904529A, NVP-ADW742, BMS-536924, Ceritinib (LDK378), AG-1024, GSK1838705A, BMS-754807, PQ 401, ZD3463, Nordihydroguaiaretic acid (NDGA), NT157, Insulin (human), Ceritinib dihydrochloride, Dioscoreae Nipponicae Rhizoma Extract, MID-1, Brigatinib (AP26113), Picropodophyllin (PPP), MSDC-0160, Insulin Degludec, Chromium picolinate, SBI-477, XL228, and combination thereof.

In some embodiments, the therapeutic agent comprises neuropilin-associated receptor inhibitors. In some embodiments, the neuropilin-associated receptor inhibitors comprise receptor tyrosine kinase, receptor serine/threonine kinase, G-protein coupled receptors, ion channel receptors, CXCR, and immune checkpoint modulators.

In some embodiments, the therapeutic agent comprises PARP inhibitors. In some embodiments, the PARP inhibitor is selected from the group consisting of PJ34 HCl, AZD2461, Olaparib (AZD2281), Veliparib (ABT-888), XAV-939, Rucaparib (AG-014699) phosphate, Iniparib (BSI-201), Talazoparib (BMN 673), AG-14361, 3-Aminobenzamide, A-966492, Niraparib (MK-4827), UPF 1069, ME0328, Licochalcone D, DR2313, MN 64, 4′,5,7-Trimethoxyflavone, Rucaparib, M2912, GeA-69, BYK204165, BGP-15 2HC1, Atamparib (RBN-2397), Venadaparib (IDX-1197), Niraparib (MK-4827) tosylate, NU1025, Rucaparib Camsylate, Berberine chloride (NSC 646666), Pamiparib (BGB-290), Fluzoparib (SHR-3162), G007-LK, NVP-TNKS656, Berberine chloride hydrate, HI-TOPK-032, Stenoparib (E7449), 4-Hydroxyquinazoline, NMS-P118, WIKI4, RBN012759, AZD5305, AZD-9574, RK-287107, Benzamide, JW55, Picolinamide, and combination thereof.

In some embodiments, the therapeutic agent comprises Raf inhibitors. In some embodiments, the Raf inhibitor is selected from the group consisting of Vemurafenib (PLX4032), B-Raf inhibitor 1 (Compound 13) dihydrochloride, Raf inhibitor 1, Raf inhibitor 2, Sorafenib (BAY 43-9006) tosylate, PLX-4720, Dabrafenib (GSK2118436), Regorafenib (BAY 73-4506), Doramapimod (BIRB 796), GDC-0879, RAF265 (CHIR-265), AZ 628, NVP-BHG712, SB590885, ZM 336372, Sorafenib (BAY 43-9006), GW5074, TAK-632, Agerafenib (RXDX-105), Encorafenib (LGX818), BAW2881 (NVP-BAW2881), PLX8394, TBAP-001, Regorafenib Hydrochloride, MCP110, Naporafenib (LXH254), B-Raf IN 1, Donafenib (Sorafenib D3), CCT196969, RAF709, Lifirafenib (BGB-283), L-779450, PLX7904, LY3009120, Dabrafenib Mesylate, R05126766 (CH5126766), AZ304, Belvarafenib (HM95573), Regorafenib (BAY-734506) Monohydrate, Tovorafenib (MLN2480), and combination thereof.

In some embodiments, the therapeutic agent comprises autophagy activators. In some embodiments, the autophagy activator is selected from the group consisting of Enzalutamide (MDV3100), Obatoclax Mesylate (GX15-070), SRT1720 HCl, Fulvestrant (ICI-182780), Bicalutamide (ICI-176334), Resveratrol (SRT501), Colforsin, Rosiglitazone (BRL-49653) maleate, Rosiglitazone (BRL 49653), Mifepristone (RU486), Purmorphamine, Clemastine (HS-592) fumarate, GW4064, Chloroquine diphosphate, Ivermectin (MK-933), Loperamide HCl, Melatonin (NSC 113928), Methylprednisolone (NSC-19987), Clonidine HCl, Flubendazole, Fenofibrate (NSC-281 319), Montelukast Sodium, LYN-1604, EN6, Eprenetapopt (APR-246), 3BDO, MHY1485, Methylprednisolone Acetate, QX77, Anisomycin, β-Elemene, Spermidine trihydrochloride, Troglitazone (CS-045), Corynoxine, Obeticholic Acid, SMER28, BC1618, Monomethyl Fumarate, PCNA-I1, CA77.1, Spermidine, Xylitol, Isorhychophylline, MPP+ iodide, and combination thereof.

In some embodiments, the therapeutic agent comprises autophagy inhibitors. In some embodiments, the autophagy inhibitor is selected from the group consisting of MK-2206 2HC1, Bortezomib (PS-341), Olaparib (AZD2281), Vemurafenib (PLX4032), Vorinostat (SAHA), ABT-737, Y27632 2HC1, Dactolisib (BEZ235), Sorafenib (BAY 43-9006) tosylate, Dasatinib (BMS-354825), Rapamycin (AY-22989), Crizotinib (PF-02341066), Erlotinib (OSI-774) HCl, Everolimus (RAD001), Gefitinib (ZD1839), Veliparib (ABT-888), Entinostat (MS-275), BI 2536, Pictilisib (GDC-0941), Laduviglusib (CHIR-99021) HCl, LY294002, Trametinib (GSK1120212), Ruxolitinib (INCB018424), Panobinostat (LBH589), Imatinib (ST1571) Mesylate, KU-55933 (ATM Kinase Inhibitor), Alisertib (MLN8237), Afatinib (BIBW2992), U0126-EtOH, Idelalisib, Tozasertib, AZD8055, Saracatinib (AZD0530), Paclitaxel (NSC 125973), SP600125, Ponatinib (AP24534), Tanespimycin (17-AAG), YM155 (Sepantronium Bromide), DAPT (GSI-IX), Cisplatin (NSC 119875), Imatinib (STI571), Nilotinib (AMN-107), Temsirolimus (CCI-779), PI-103, Luminespib (NVP-AUY922), Vandetanib (ZD6474), Gemcitabine (LY-188011) HCl, Mocetinostat (MGCD0103), Regorafenib (BAY 73-4506), SRT1720 HCl, Pazopanib HCl (GW786034 HCl), Bosutinib (SKI-606), Cediranib (AZD2171), Belinostat (PXD101), GSK690693, SB202190 (FHPI), Carfilzomib (PR-171), Fulvestrant (ICI-182780), SB216763, SU11274, Linifanib (ABT-869), Pemetrexed (LY-231514) disodium, Torkinib (PP242), Etoposide (VP-16), Wortmannin (KY 12420), LY2109761, Danusertib (PHA-739358), Silmitasertib (CX-4945), Venetoclax (ABT-199), Flavopiridol (L86-8275), SGI-1776 free base, Lapatinib (GW-572016), Vincristine (NSC-67574) sulfate, Temozolomide (CCRG 81045), Tacrolimus (FK506), Metformin HCl, Fasudil (HA-1077) HCl, Oxaliplatin (NSC 266046), Rabusertib (LY2603618), 3-Methyladenine (3-MA), Flavopiridol (L86-8275) HCl, (+)-JQ1, Zoledronic acid (ZOL 446), Momelotinib (CYT387), Ixazomib Citrate (MLN9708) Analogue, Tamoxifen (ICI 46474) Citrate, Letrozole (CGS 20267), Topotecan (NSC609699) HCl, Torin 1, Omipalisib (GSK2126458), Degrasyn (WP1130), 2-Methoxyestradiol (2-MeOE2), Azacitidine (5-Azacytidine), BX-795, OSI-027, Ixazomib (MLN2238), TWS119, Apitolisib (GDC-0980), BI-D1870, Resveratrol (SRT501), Dexamethasone (MK-125), Cytarabine (U-19920A), Simvastatin (MK 733), Vistusertib (AZD2014), CCT128930, Idarubicin HCl, Gemcitabine (LY-188011), Verteporfin (CL 318952), PF-4708671, Torin 2, Streptozotocin (STZ), H 89 2HC1, Brefeldin A, Geldanamycin (NSC 122750), MK-5108 (VX-689), Colforsin, Valproic Acid (NSC 93819) sodium salt, Pitavastatin (NK-104) calcium, Dorsomorphin (Compound C) 2HC1, Lovastatin (MK-803), Rosiglitazone (BRL-49653) maleate, Necrostatin-1, Pazopanib, Nocodazole (R17934), Daporinad, Clofarabine, Cabazitaxel (XRP6258), Atorvastatin Calcium, (R)-(−)-Gossypol acetic acid, Pifithrin-α (PFTa) HBr, SN-38, GSK343, BIX 01294, Nutlin-3a, Tigecycline (GAR-936), STF-62247, Binimetinib (MEK162), Itraconazole (R 51211), Milciclib (PHA-848125), Sorafenib (BAY 43-9006), 10058-F4, YM201636, C646, Hydroxyurea (NSC-32065), Bardoxolone Methyl, Sodium butyrate, PR-619, Linagliptin (BI-1356), Niclosamide (BAY2353), Nitazoxanide (NSC 697855), UNC1999, Heparin sodium, Dynasore, Curcumin, Celastrol (NSC 70931), GSK2606414, Honokiol (NSC 293100), Laduviglusib (CHIR-99021), GANT61, Omeprazole, Amiodarone (NSC 85442) HCl, Dexamethasone Sodium Phosphate, Aspirin (NSC 27223), AZD3463, GSK2656157, Carbamazepine, Bafilomycin Al (Baf-Al), Azithromycin (CP-62993), Nimodipine, IU1, Sulfasalazine (NSC 667219), Pifithrin-μ, PF-543 hydrochloride, Tubastatin A, Nitrendipine, DC661, KB-R7943 mesylate, Tubastatin A TFA, PFK15, Nordihydroguaiaretic acid (NDGA), Nilotinib hydrochloride monohydrate, Salirasib, Neferine, Emetine hydrochloride, SBI-0206965, Apatinib, EAD1, Lanatoside C, Ruxolitinib Phosphate, Losmapimod (GW856553X), STO-609, IITZ-01, Vacuolin-1, Entrectinib (RXDX-101), Sunitinib (SU11248), Valproic acid (VPA), URMC-099, Spautin-1, Erlotinib (OSI-774), Pemetrexed Disodium Hydrate, Crizotinib hydrochloride, Atorvastatin, Berberine chloride (NSC 646666), Quercetin (NSC 9221), VLX600, Afatinib (BIBW2992) Dimaleate, FL-411, Pemetrexed, Autophinib, Cryptotanshinone, Nortriptyline hydrochloride, Dihydroartemisinin (DHA), KN-93 Phosphate, Telaglenastat (CB-839), Purvalanol A, Brevilin A, Chloroquine (NSC-187208), PFK158, Sulfacetamide sodium salt hydrate, OTS964, Sinomenine hydrochloride, MRT67307 HCl, Lys05, Sulfacetamide Sodium, Resatorvid (TAK-242), DMH1, Hydroxychloroquine Sulfate (NSC 4375), MRT68921 HCl, PHY34, Vinorelbine ditartrate (KW-2307), CA-5f, VPS34-IN1, DMOG, ICCB-19 hydrochloride, AS1842856, SAR405, PIK-Ill, ULK-101, Vinblastine (NSC-49842) sulfate, SP2509, ROC-325, Codonopsis Pilosula Extract, ABTL-0812, ML-9 HCl, AZD1208, Lucanthone, Leonurine, VER155008, RA-190, LY3009120, 4-Phenylbutyric acid (4-PBA), NSC 185058, E260, Daurisoline, Dorsomorphin (Compound C), Tamoxifen (ICI 46474), Deferoxamine mesylate (Ba 33112), Pepstatin A, Trametinib DMSO solvate, and Concanavalin A.

In some embodiments, the therapeutic agent comprises mTOR inhibitors. In some embodiments, the mTOR inhibitor is selected from the group consisting of mTOR inhibitor-1, Everolimus (RAD001), KU-0063794, Dactolisib (BEZ235), Rapamycin (AY-22989), AZD8055, Temsirolimus (CCI-779), PI-103, NU7441 (KU-57788), Torkinib (PP242), Ridaforolimus (Deforolimus, MK-8669), Sapanisertib (MLN0128), Voxtalisib (XL765) Analogue, Torin 1, Omipalisib (GSK2126458), OSI-027, PF-04691502, Apitolisib (GDC-0980), GSK1059615, Gedatolisib (PKI-587), WYE-354, Vistusertib (AZD2014), Torin 2, WYE-125132 (WYE-132), BGT226 (NVP-BGT226) maleate, Palomid 529 (P529), PP121, WYE-687, Nitazoxanide (NSC 697855), WAY-600, ETP-46464, GDC-0349, XL388, 4EGI-1, JR-AB2-011, Rotundic acid, Lanatoside C, Compound 401, Astragaloside IV, Ginkgolide K, CC-115, Zotarolimus (ABT-578), Paxalisib (GDC-0084), CZ415, SF2523, Bimiralisib (PQR309), Voxtalisib (XL765), Chrysophanic Acid, Onatasertib (CC 223), 3-Hydroxyanthranilic acid, Samotolisib (LY3023414), MTI-31, ABTL-0812, PQR620, MHY-1685, GNE-477, GNE-493, and combination thereof.

In some embodiments, the therapeutic agent comprises PI3K activators and PI3K inhibitors. In some embodiments, the PI3K activator is selected from the group consisting of Demethyl-Coclaurine, Cinobufagin, Resibufogenin, 740 Y-P (PDGFR 740Y-P), Erucic acid, Amarogentin, YS-49, and combination thereof. In some embodiments, the PI3K inhibitor is selected from the group consisting of PI-103, XL147 analogue, 3-Methyladenine (3-MA), Apitolisib (GDC-0980), TG100713, Taselisib (GDC 0032), Dactolisib (BEZ235), Pictilisib (GDC-0941), LY294002, Idelalisib, Buparlisib (BKM120), NU7441 (KU-57788), TGX-221, IC-87114, Wortmannin (KY 12420), ZSTK474, Alpelisib (BYL719), AS-605240, PIK-75 HCl, Rigosertib (ON-01910), A66, Voxtalisib (XL765) Analogue, Omipalisib (GSK2126458), PIK-90, AZD6482, PF-04691502, GSK1059615, Duvelisib (IPI-145), Gedatolisib (PKI-587), TG100-115, AS-252424, NU7026, BGT226 (NVP-BGT226) maleate, Fimepinostat (CUDC-907), PIK-294, AS-604850, GSK2636771, Copanlisib (BAY 80-6946), YM201636, CH5132799, CAY10505, PIK-293, PKI-402, VS-5584 (SB2343), KU-0060648, CZC24832, Demethyl-Coclaurine, Oroxin B, Homosalate, AMG319, Cinobufagin, Resibufogenin, Hispidulin, GSK2292767, Lanatoside C, Zeaxanthin, Disitertide (P144), Cafestol, Paxalisib (GDC-0084), SKI-V, MTX-211, Seletalisib (UCB-5857), Trichosanthis Pericarpium Extract, Dioscoreae Nipponicae Rhizoma Extract, GDC-0326, 740 Y-P (PDGFR 740Y-P), PIK-108, Parsaclisib (INCB050465) Hydrochloride, HS-173, SF2523, leniolisib (CDZ 173), Lupenone, Serabelisib (TAK-117), Eganelisib (IPI-549), Quercetin (NSC 9221), Bimiralisib (PQR309), VPS34 inhibitor 1 (Compound 19), 1HMT-PI3K6-372, Voxtalisib (XL765), Autophinib, GNE-317, (E)-Akt inhibitor-IV, Notoginsenoside RI, Tenalisib (RP6530), Solasodine, Gallein, Pectolinarin, Inavolisib (GDC-0077), SRX3207, a-Linolenic acid, umbralisib (TGR-1202), acalisib (GS-9820), ME-401, 3-Hydroxyanthranilic acid, Nemiralisib, Samotolisib (LY3023414), VPS34-IN1, Ailanthone, Tripterygium wilfordii Extract, IPI-3063, SAR405, PIK-III, IPI-3063, Parsaclisib (INCB050465), Quercetin Dihydrate, Pilaralisib (XL147), SPP-86, AZD8835, Trigonelline, Deguelin, Selective PI3K6Inhibitor 1 (compound 7n), Loureirin A, PF-4989216, AZD8186, GNE-477, GNE-493, and combination thereof.

In some embodiments, the therapeutic agent comprises proteasome inhibitors. In some embodiments, the proteasome inhibitor is selected from the group consisting of Salinosporanide A (NPI-0052), Bortezomib (PS-341), MG132, Carfilzomib (PR-171), Ixazomib Citrate (MLN9708), Ixazomib (MLN2238), ONX-0914 (PR-957), Oprozomib (ONX 0912), Delanzomib (CEP-18770), Celastrol (NSC 70931), Epoxomicin (BU-4061T), Shikonin (C.I. 75535), VR23, Isoginkgetin, RA-190, and PI-1840.

In some embodiments, the therapeutic agent comprises JNK inhibitors. In some embodiments, the JNK inhibitor is selected from the group consisting of JNK Inhibitor VIII, JNK Inhibitor IX, JNK-IN-8, BI-78D3, SP600125, Doramapimod (BIRB 796), Metformin HCl, DB07268, 3′-Hydroxypterostilbene, KB-R7943 mesylate, JNK-IN-7, Loureirin B, Astragaloside IV, Cucurbitacin IIb, Trans-Zeatin, Ezatiostat, IQ 3, Berberine chloride (NSC 646666), SU3327, Bentamapimod (AS602801), Indirubin-3′-oxime, Falcarindiol, Tanzisertib (CC-930), NDMC101, Mulberroside A, C-401 Hydrochloride, RPI-1, Ginsenoside Re, IQ-1S, Urolithin B, and combination thereof.

In some embodiments, the therapeutic agent comprises NF-κB inhibitors. In some embodiments, the NF-κB inhibitor is selected from the group consisting of Phospho-IKB alpha (S32) Rabbit Recombinant mAb, Ophiopogonin D, Cornuside, Rubiadin 1-methyl ether, SM-7368, NF-κB-IN-1, Chitosan oligosaccharide, Cynanchi Atrati Extract, IAXO-102, Adjudin, CBL0137, IQ 3, Hypaphorine, Isoliquiritin apioside, Jaceosidin, Bortezomib (PS-341), Urolithin B, Sulfasalazine (NSC 667219), Acetylcysteine (N-acetylcysteine), Erdosteine, Curcumin, Andrographolide, Dihydroartemisinin (DHA), Indole-3-carbinol, Magnolol, (−)-Parthenolide, Evodiamine, Chondroitin sulfate, TPCA-1, Sodium salicylate, Methylthiouracil, Articaine HCl, L-Quebrachitol, UCB-9260, Zeaxanthin, Tectochrysin, Myrislignan, Gardenoside, Caulophylline (N-Methylcytisine), Demethyleneberberine, 3-Hydroxyanthranilic acid, (E/Z)-IT-603, Triptolide (PG490), Sarsasapogenin, Berbamine dihydrochloride, Pyrrolidinedithiocarbamate ammonium, Stachydrine hydrochloride, Sodium Aescinate, Stachydrine, Tyrosol, Mangiferin, Scutellarin, Ginsenoside Re, Dehydroevodiamine, (E)-Cardamonin, Muscone, Guaiacol, Curcumenol, Schisantherin A, Hederagenin, Astragaloside IV, Ginsenoside Rgl, Ginsenoside Rbl, Ginsenoside Rd, 4′-Methoxyresveratrol, (+)-α-Lipoic acid, Sodium 4-Aminosalicylate, IMM-H007, Diethylmaleate, 4-Hydroxychalcone, Benfotiamine, QNZ (EVP4593), 4′-Hydroxychalcone, Neferine, Vanillic acid, Hyperoside, Chelidonic acid, Ethyl caffeate, Sulforaphane, (R)-(−)-Ibuprofen, SN50, C25140, INH14, Licochalcone D, SC75741, JSH-23, Caffeic Acid Phenethyl Ester, Rocaglamide, APX-3330, DTP3, Omaveloxolone (RTA-408), Bardoxolone Methyl, Shikonin (C.I. 75535), TAK-243 (MLN7243), CBL0137 HCl, Withaferin A, NIK SMI1, Alobresib (GS-5829), anthatin, Maslinic acid, Mulberroside A, Eleutheroside E, Berbainine, Engeletin, Aristolochic acid A, Ginsenoside Rb3, 8-O-acetyl shanzhiside methyl ester, Dauricine, 2′,5′-Dihydroxyacetophenone, (+)-Praeruptorin A, Homoplantaginin, Madecassic acid, BTYNB, and combination thereof.

In some embodiments, the therapeutic agent comprises HSP90 inhibitors. In some embodiments, the HSP90 inhibitor is selected from the group consisting of Luminespib (NVP-AUY922), Tanespimycin (17-AAG), Alvespimycin (17-DMAG) HCl, Ganetespib (STA-9090), Elesclomol (STA-4783), B11B021, Tamoxifen (ICI 46474) Citrate, Onalespib (AT13387), NVP-BEP800, Geldanamycin (NSC 122750), SNX-2112 (PF-04928473), PF-04929113 (SNX-5422), KW-2478, XL888, Pifithrin-μ, NMS-E973, Zelavespib (PU-H71), Teprenone, Dimethylenastron, Apoptozole, KRIBB11, Pseudolaric Acid A, TRC051384, DTHIB, Rocaglamide, KNK437, VER-49009, HA 15, Pimitespib (TAS-116), CH5138303, VER-50589, YUM70, Triptolide (PG490), VER155008, JG98, Tamoxifen (ICI 46474), NPX800, HSP990 (NVP-HSP990), and combination thereof.

In some embodiments, the therapeutic agent comprises E3 ligase inhibitors. In some embodiments, the E3 ligase inhibitor is selected from the group consisting of Lenalidomide (CC-5013), Pomalidomide (CC-4047), Thalidomide (K17), NSC 207895, TAME, PRT4165, CC-885, dCBP-1, Iberdomide (CC220), BC-1215, CC-90009, Avadomide (CC-122), VL285, (S,R,S)-AHPC (MDK7526), Smurfl-IN-A01, (S,R,S)-AHPC-PEG4-NH2 hydrochloride, SZL P1-41, VH298, Thalidomide-OH, MuRF1-IN-1, Skp2 inhibitor Cl (SKPin C1), GMB-475, Mezigdomide (CC-92480), Homo-PROTAC cereblon degrader 1, THAL-SNS-032, NSC232003, Apcin, Thalidomide-O—COOH (Cereblon ligand 3), and combination thereof.

In some embodiments, the therapeutic agent comprises KRAS inhibitors. In some embodiments, the KRAS inhibitor is selected from the group consisting of MRTX1133, Sotorasib (AMG510), Adagrasib (MRTX849), LC-2, Deltarasin, BAY-293, ARS-1620, BI-2852, ARS-853 (ARS853), BI-3406, ASP2453, Sotorasib (AMG510) racemate, Pan-RAS-IN-1, MRTX-1257, Zoledronic acid (ZOL 446), Lonafarnib (SCH66336), K-Ras (G12C) inhibitor 9, Salirasib, Alamandine, (Rac)-Antineoplaston A10, K-Ras-IN-1, MCP110, 61105, K-Ras (G12C) inhibitor 12, Kobe0065, K-Ras (G12C) inhibitor 6, BQU57, Kobe2602, NAV-2729, Antineoplaston A10, Fendiline hydrochloride, KRpep-2d, Perillyl alcohol, RBC8, KY1220, CID-1067700, Zoledronic acid monohydrate, and combination thereof.

In some embodiments, the protein that the therapeutic agent targets and inhibits is the same protein that the target protein degrader binds to and degrades.

In certain embodiments, use of a target protein degrader and an EGFR inhibitor in the manufacture of medicament for treating a cancer in a subject when administered in combination with an EGFR inhibitor.

In some embodiments, the therapeutic agent is an EGFR inhibitor, and the target protein is the EGFR degrader. In some embodiments, EGFR inhibitor is Osimertinib. In some embodiments, the cancer is Osimertinib-resistant NSCLC.

In some embodiments, the target protein degrader is intraperitoneally administered.

In some embodiments, the methods comprise administering to the patient an amount of the target protein degrader that increases the therapeutic activity of the therapeutic agent by at least 2-fold. The method of claim 1, comprising administering to the patient an amount of the target degrader that increases the therapeutic activity of the therapeutic agent by at least 5-fold. The method of claim 1, comprising administering to the patient an amount of the target degrader that increases the therapeutic activity of the therapeutic agent by at least 10-fold. The method of claim 1, comprising administering to the patient an amount of the target degrader that increases the therapeutic activity of the therapeutic agent by at least 20-fold.

In some embodiments, the cancer is resistant to an immunotherapeutic antibody. In certain embodiments, the immunotherapeutic antibody comprises anti-EGFR monoclonal antibodies. In certain embodiments, the anti-EGFR monoclonal antibody comprises amivantamab, cetuximab, depatuxizumab, depatuxizumab mafodotin, duligotuzumab, futuximab, GC 1118, Imagatuzumab, matuzumab, necitumumab, nimotuzumab, panitumumab, zalutumumab, and HumMR1.

In some embodiments, the subject is a human.

The present disclosure further provides various methods of administering a pharmaceutical composition comprising the bispecific antibody to subjects. In some embodiments, the administering is intravenous. In some embodiments, the administering is intraperitoneal, intrathecal, intraventricular, or intraparenchyal.

The following figures are provided by way of example and are not intended to limit the scope of the invention.

### BRIEF DESCRIPTION OF FIGURES

FIG. 1 is a schematic illustrating the acquisition of resistance following targeted therapies.

FIG. 2 illustrates that existing EGFR blockers fail to achieve a sustained response due to lack of EGFR degradation.

FIG. 3 illustrates the enhanced anti-tumor activity of the combination treatment with Osimertinib and the EGFR degrader in Osimertinib-resistant mouse xenograft (H1975-OR). Mouse H1975-OR xenograft model was treated with EGFR signal blocking antibody Panitumumab (10 mg/kg) or EGFR degrader (13.74 mg/kg) in combination with high dose Osimertinib (10 mg/kg). Tumor volume was quantitated to determine the therapeutic effects by the EGFR degrader.

FIG. 4 illustrates that co-treatment with a target protein and a target protein degrader prevents or minimizes the acquired resistance to the target protein inhibitor in a target protein-resistant cancer.

FIGS. 5A-5B are graphs showing results from H1975-NRP1 OE xenograft studies using the indicated treatments. FIG. 5A shows mean tumor volume over time. FIG. 5B shows probability of survival over time.

FIGS. 6A-6B are graphs showing results from H1975-HGF xenograft studies using the indicated treatments. FIG. 6A shows results for combination with Osimertinib. FIG. 6B shows results for combination with Osimertinib and/or Crizotinib.

FIG. 7 is a graph showing results from a mutant KRAS xenograft studies using the indicated treatments. Results show mean tumor volume over time.

### DETAILED DESCRIPTION

Provided herein are methods and compositions for treating cancer, and preventing resistance to an antiproliferative drug or enhancing its therapeutic effect in a subject. Using Osimertinib-resistant H1975 xenograft mouse model, we have found that treatment with an EGFP degrader, EGFP×NRP1 antibody, can sensitize Osimertinib-resistant cancer to continued treatment with Osimertinib. The EGFP degrader, (EGFR×NRP1 antibody) may be used alone or in combination with any EGFR inhibitor to prevent or minimize the acquired resistance to the cancer drug. We demonstrate that the combination treatment with the target protein degrader and the target protein inhibitor is an efficient therapeutic tool to treat cancer that already has or would develop the resistance to a target protein inhibitor.

### 1. Definitions

Unless defined otherwise, all technical and scientific terms used herein have the same meaning as commonly understood by one of ordinary skill in the art to which this disclosure belongs. Accordingly, the following terms are intended to have the following meanings:

As used herein, the singular form “a”, “an” and “the” includes their plural references unless the context clearly dictates otherwise.

As used herein, the terms “comprise(s),” “include(s),” “having,” “has,” “can,” “contain(s),” and variants thereof, as used herein, are intended to be open-ended transitional phrases, terms, or words that do not preclude the possibility of additional acts or structures.

As used herein, “administration” of a disclosed polypeptide encompasses the delivery to a subject of a polypeptide or composition of the present invention, as described herein, or a prodrug or other pharmaceutically acceptable derivative thereof, using any suitable formulation or route of administration, e.g., as described herein.

As used herein, the term “and/or,” when used in a list of two or more items, means that any one of the listed items can be employed by itself, or any combination of two or more of the listed items can be employed.

As used herein, the “cancer” refers to a neoplasm or tumor resulting from abnormal and uncontrolled growth of cells. Cancer may also be referred to as a cellular-proliferative disease. Cancer may include different histological types, cell types, and different stages of cancer, such as, for example, primary tumor or metastatic growth. Cancer may include, for example, breast cancer, cholangiocellular carcinoma, colorectal cancer, endometriosis, esophageal cancer, gastric cancer, diffused type gastric cancer, pancreatic cancer, renal carcinoma, soft tissue tumor, testicular cancer, cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma, non-small cell lung cancer (NSCLC), small cell lung cancer (SCLC); Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinorna, glucagonoma, gastrinoma, carcinoid tumors, vipoma), small bowel (adenocarcinorna, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defornians), meninges (meningioma, meningiosarcoma, gliomatosis), brain (astrocytoma, medulloblastoma, glioma, ependymoma, germinoma [pinealoma], glioblastoma, glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian cancer, ovarian carcinoma [serous cystadenocarcinoma, mucinous cystadenocarcinoma, unclassified carcinoma], granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia |acute and chronic|, acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma], CML; Skin: melanoma, malignant melanoma, basal cell carcinoma, squamous cell carcinoma, Karposi's sarcoma, moles, dysplastic nevi, lipoma, angioma, dermatofibroma, keloids, psoriasis; and Adrenal glands: neuroblastoma.

In some embodiments, the cancer comprises non-small cell lung cancer (NSCLC). In some embodiments, the cancer is resistant to a therapy. In some embodiments, the cancer is not resistant to a therapy.

The term “carrier” refers to the vehicle used in the formulation of a composition and can be composed of multiple excipients.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

The term “excipient” as used herein refers to any pharmacologically inactive, natural, or synthetic, component or substance that is formulated alongside (e.g., concomitantly), or subsequently to, the active ingredient of the present invention. In some embodiments, an excipient can be any additive, adjuvant, binder, bulking agent, carrier, coating, diluent, disintegrant, filler, glidant, lubricant, preservative, vehicle, or combination thereof, with which a recombinant polypeptide of the present invention can be administered, and or which is useful in preparing a composition of the present invention. Excipients, include any such materials known in the art that are nontoxic and do not interact with other components of a composition. In some embodiments, excipients can be formulated alongside a recombinant polypeptide when preparing a composition for the purpose of bulking up compositions (thus often referred to as bulking agents, fillers, or diluents). In other embodiments, an excipient can be used to confer an enhancement on the active ingredient in the final dosage form, such as facilitating absorption and/or solubility. In yet other embodiments, an excipient can be used to provide stability, or prevent contamination (e.g., microbial contamination). In other embodiments, an excipient can be used to confer a physical property to a composition (e.g., a composition that is a dry granular, or dry flowable powder physical form). Reference to an excipient includes both one and more than one such excipients. Suitable pharmaceutical excipients are described in Remington's Pharmaceutical Sciences, by E. W. Martin, the disclosure of which is incorporated herein by reference in its entirety.

The term “inhibitor” refers to a compound that inhibits or reduces an activity of a polypeptide. An inhibitor may indirectly or directly bind a polypeptide and inhibit the activity of the polypeptide, including binding activity or catalytic activity. For example, an inhibitor may prevent expression of a polypeptide, or inhibit the ability of a polypeptide to mediate the binding of the polypeptide to a ligand. An “allosteric inhibitor” refers to a compound that binds to a polypeptide at a secondary site, distinct from the primary ligand binding site, and inhibits or reduces an activity of the polypeptide. The terms “inhibit” or “inhibiting” mean that an activity is decreased or prevented in the presence of an inhibitor as opposed to in the absence of the inhibitor. The term “inhibition” refers to the reduction or down regulation of a process or the elimination of a stimulus for a process, which results in the absence or minimization of the expression or activity of a biomolecule or polypeptide. Inhibition may be direct or indirect. Inhibition may be specific, that is, the inhibitor inhibits a biomolecule or polypeptide and not others.

As used herein, an “EGFR×NRP1 bispecific antibody” or “anti-EGFR×NRP1 bispecific antibody” (for use as a targeted protein degrader) is a bispecific binding molecule, which comprises two different antigen binding domains, one of which binds specifically to the antigen EGFR1 and one of which binds specifically to NRP1. Similar nomenclature is used throughout for other bispecific binding molecules that bind targets other than EGFR, such as “HER2×NRP1 bispecific antibody” or “cMET×NRP1 bispecific antibody” to describe a bispecific binding molecule comprising one antigen binding domain that binds specifically to HER2 or cMET, respectively, and the other antigen binding domain that binds specifically to NRP1.

As used herein, the terms “receptors tyrosine kinase” or “Receptor Tyrosine Kinase” or “RTK” refers to proteins that are receptors (i.e., bind a ligand) and that phosphorylate a tyrosine residue(s).

As used herein the terms “non-receptors tyrosine kinase” or “Non-Receptor Tyrosine Kinase” or “non-RTK” refers to proteins that are not RTKs, i.e., proteins that are not receptors and/or do not phosphorylate a tyrosine residue(s).

As used herein, “treatment”, “treat”, or “treating”, are used interchangeably herein, and refer to an approach for obtaining a therapeutic benefit. A therapeutic benefit is determined by whether the tumor shrinks, stays the same size, or an increase in progression free survival time compared to placebo. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques.

The term “pharmaceutically acceptable salts” is meant to include salts of the active bispecific antibodies that are prepared with relatively nontoxic acids or bases, depending on the particular substituent found on the bispecific antibodies described herein.

The terms “polypeptide,” “peptide” and “protein” are used interchangeably herein to refer to a polymer of amino acid residues. “Polynucleotide” as used herein can be single stranded or double stranded, or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods. The term “reduce” or other forms of the word, such as “reducing” or “reduction,” generally refers to the lowering of an event or characteristic (e.g., one or more symptoms, or the binding of one protein to another). It is understood that this is typically in relation to some standard or expected value, in other words it is relative, but that it is not always necessary for the standard or relative value to be referred to.

The “recombinant” when used with reference, for example, to a cell, nucleic acid, polynucleotide, protein, or vector, indicates that the cell, nucleic acid, polynucleotide protein, or vector, has been modified by the introduction of a heterologous nucleic acid or protein or the alteration of a native polynucleotide or protein, or that the cell is derived from a cell so modified. Thus, for example, recombinant cells express genes that are not found within the native (non-recombinant) form of the cell or express native genes that are otherwise abnormally expressed, under expressed, or not expressed at all. For example, the term “recombinant DNA molecule” as used herein refers to a DNA molecule which is comprised of segments of DNA joined together by means of molecular biological techniques. The term “recombinant protein” or “recombinant polypeptide” as used herein refers to a protein molecule which is expressed from a recombinant DNA molecule or recombinant polynucleotide.

By “specifically binds,” it is generally meant that an agent or polypeptide binds to a target when it binds to that target more readily than it would bind to a random, unrelated target.

A “subject,” as used herein, can refer to any animal with a cancer, e.g., a mammal, such as an experimental animal, a farm animal, pet, or the like. In some embodiments, the animal is a primate, preferably a human. As used herein, the terms “subject” and “subject” are used interchangeably. The terms “subject” and “subject” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee, and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a “human”.

As used herein, the term “synergistic” refers to a combination of a polypeptide of the invention and another therapy (e.g., a prophylactic or therapeutic agent), which is more effective than the additive effects of the therapies.

A “therapeutically effective amount,” or “effective dosage,” or “effective amount” as used interchangeably herein unless otherwise defined, means a dosage of an agent or drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual. This term as used herein may also refer to an amount effective at bringing about a desired in vivo effect in a subject. A therapeutically effective amount may be administered in one or more administrations (e.g., the composition may be given as a preventative treatment or therapeutically at any stage of disease progression, before or after symptoms, and the like), applications, or dosages, and is not intended to be limited to a particular formulation, combination, or administration route. It is within the scope of the present disclosure that the drug may be administered at various times during the course of treatment of the subject. The times of administration and dosages used will depend on several factors, such as the goal of treatment (e.g., treating v. preventing), condition of the subject, etc. and can be readily determined by one skilled in the art. A therapeutically effective amount is also one in which any toxic or detrimental effects of substance are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The terms “treat,” “treated,” or “treating” as used herein refers to a therapeutic wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment. The terms “treat,” “treated,” or “treating” may include preventing, suppressing, repressing, ameliorating, or completely eliminating the disease.

Preventing the disease may involve administering a composition of the present invention to a subject prior to onset of the disease. Suppressing the disease may involve administering a composition of the present invention to a subject after induction of the disease but before its clinical appearance. Repressing or ameliorating the disease may involve administering a composition of the present invention to a subject after clinical appearance of the disease.

The term “variant” as used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequence substantially identical thereto.

As used herein, the term “percent identity” between two sequences (e.g., amino acid or nucleotide sequences) refers to the percentage of positions (out of a possible 100%) that when optimally aligned and compared, are identical (with appropriate insertions or deletions for optimal alignment). The percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences.

The comparison of sequences and determination of percent identity between two sequences can be accomplished using mathematical algorithm, as described in the non-limiting examples below. Methods and algorithms for determining the % homology between two protein sequences are well established in the art.

For example, the percent identity between two amino acid sequences can be determined using the Needleman and Wunsch ((1970) J. Mol. Biol. (48):444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. Furthermore, a protein amino acid sequence can be used as a “query sequence” to perform a search against public databases to, for example, identify related sequences. Such searches can be performed using the XBLAST programs (version 2.0) of Altschul, et al. (1990) J Mol. Biol. 215:403-10. BLAST protein searches can be performed with the XBLAST program, score=50, wordlength=3 to obtain amino acid sequences homologous to the protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used.

All patent applications, patents, and printed publications referred to herein are incorporated by reference in their entirety to the same extent as if each individual publication, patent, or patent application was specifically and individually indicated to be incorporated by reference in its entirety. And all patent applications, patents, and printed publications cited herein are incorporated herein by reference in their entireties, except for any definitions, subject matter disclaimers, or disavowals, and except to the extent that the incorporated material is inconsistent with the express disclosure herein, in which case the language in this disclosure controls.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate and are provided for the description.

### 2. Target Protein Degrader and EGFR Degrader

Further provided herein is a target protein degrader. The target protein degrader comprises the target protein binding domain and a neuropilin binding domain. The target protein binding domain and a neuropilin binding domain of the target protein degrader specifically bind to the target protein and NRP1 and the internalized target protein is subsequently degraded via lysosomes. The target protein degrader comprises any heavy chain polypeptides of SEQ ID NOs: 2 to 11 and a light chain polypeptide of SEQ ID NO: 12. Are also provided sequences set forth in SEQ ID NOs: 15 to 24 of nucleotides encoding the corresponding the heavy chain polypeptides of SEQ ID NOs: 2 to 11 and the sequence set forth in SEQ ID NO: 25 of nucleotide encoding the light chain polypeptide of SEQ ID NO:12. The sequences of all the polypeptides, polynucleotides, and a linker disclosed in U.S. Provisional Application No. 63/325,312 are incorporated herein.

For example, the EGFR degrader is a bispecific antibody (EGFR×NRP1 Ab) that specifically binds to EGFR and NRP1. The binding of the EGFR degrader to EGFR and NRP1 results in the degradation of EGFR. EGFR degradation by the EGFR degrader is mediated through lysosomal degradation pathway. The EGFR degrader comprises the polypeptide sequences set forth in SEQ ID NOs: 11 and 12. The sequences of polynucleotides encoding the polypeptides of SEQ ID NOs: 11 and 12 are set forth in SEQ ID NOs: 15 and 25. see Tables 1 and 2.

TABLE 1
Amino acid sequences of the EGFR degrader

| Bispecific Antibody | |
|---|---|
| Components | Amino Acid Sequence |
| SEQ ID NO: 11 | QVQLQESGPGLVKPSETLSLICTVSGGSVSSGDYYVVTWIRQSPGKGLEWIG |
| | HIYYSGNINYNPSLKSRLTISIDTSKTQFSLKLSSVTAADTAIYYCVRDRVTGAF |
| | DIWGQGTMVTVSSASTKGPSVFPLAPSSKSTSGGTAALGOCLVKDYFPEPVTV |
| | SWNSGALTSGVHTFPAVLQSSGLYSLSSVVTVPSSSLGTQTYICNVNHKPSN |
| | TKVDKKVEPKSCDKTHTCPPCPAPELLGGPSVFLFPPKPKDTLMISRIPEVTC |
| | VVVDVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSRDELTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGKGGGGSGGGGSGGGGSEV |
| | QLLESGGGLVQPGGSLRLSCAASGFTFSSYAMSWVRQAPGKGLEWVSQIS |
| | PAGGYTNYADSVKGRFTISRDNSKNTLYLQMNSLRAEDTAVYYCARGELPYF |
| | RMSKVMDVWGQGTLVTVSSGGGGSGGGGSGGGGSEIVLTQSPGTLSLSP |
| | GERATLSCRASQFLSSYLAWYQQKPGQAPRLLIYGASARASGIPDRFSGSGS |
| | GTDFTLTISRLEPEDFAVYYCQQYLASPATFGQGTKVEIK |
| SEQ ID NO: 12 | DIQMTQSPSSLSASVGDRVIITCQASQDISNYLNWYQQKPGKAPKLLIYDAS |
| | NLETGVPSRFSGSGSGTDFITTISSLQPEDIATYFCQHFDHLPLAFGGGTKVEI |
| | KRTVAAPSVFIFPPSDEQLKSGTAVVWCLLNNFYPREAKVQWKVDNALQSG |
| | NSQESVIEQDSKDSTYSLSSTLTLSKADYEKHKVYACEVTHQGLSSPVTKSFNRGEC |
| SEQ ID NO: 13 | GGGGS |

TABLE 2
Nucleotide sequences encoding EGFR degrader polypeptides

| Bispecific Antibody | |
|---|---|
| Components | Polynucleotide Sequence |
| SEQ ID NO: 24 | CAGGTTCAGCTGCAAGAGTCTGGCCCTGGCCTGGTCAAGCCTTCCGAAA |
| Encodes polypeptide | CACTGTCTCTGACCTGCACCGTGTCTGGGGGCTCTGTGTCCTCTGGCGAT |
| SEQ ID NO: 11 | TACTACTGGACCTGGATCCGGCAGTCTCCTGGCAAAGGCCTGGAATGGA |
| | TCGGCCACATCTACTACTCCGGCAACACCAACTACAACCCCAGCCTGAAG |
| | TCCCGGCTGACCATCTCCATCGACACCAGCAAGACCCAGTTCTCCCTGAA |
| | GCTGTCCTCTGTGACCGCCGCTGATACCGCCATCTACTATTGCGTGGGGG |
| | ACAGAGTGACCGGCGCCTTTGATATTTGGGGCCAGGGCACCATGGTCAC |
| | CGTGTCCAGTGCTTCTACCAAGGGACCCAGCGTGTTCCCTCTGGCTCCTT |
| | CCAGCAAGTCTACCTCTGGCGGAACAGCTGCTCTGGGCTGTCTGGTCAA |
| | GGACTACTTCCCTGAGCCTGTGACCGTGTCCTGGAATTCTGGCGCTCTGA |
| | CATCTGGCGTGCACACCTTTCCAGCTGTGCTGCAGTCTAGCGGCCTGTAC |
| | TCTCTGTCCTCCGTCGTGACCGTGCCTTCTAGCTCTCTGGGCACCCAGAC |
| | CTACATCTGCAATGTGAACCACAAGCCATCCAACACCAAGGTGGACAAG |
| | AAGGTGGAACCCAAGTCCTGCGACAAGACCCACACCTGTCCTCCATGTC |
| | CTGCTCCAGAACTGCTCGGGGGACCTTCCGTGTTCCTGTTTCCTCCAAAG |
| | CCTAAGGACACCCTGATGATCTCTCGGACCCCTGAAGTGACCTGCGTGG |
| | TGGTGGATGTGTCTCACGAGGATCCCGAAGTGAAGTTCAATTGGTACGT |
| | GGACGGCGTGGAAGTGCACAACGCCAAGACCAAGCCTAGAGAGGAACA |
| | GTACAACTCCACCTACAGAGTGGTGTCCGTGCTGACCGTGCTGCACCAG |
| | GATTGGCTGAACGGCAAAGAGTACAAGTGCAAGGTGTCCAACAAGGCC |
| | CTGCCTGCTCCTATCGAAAAGACCATCAGCAAGGCTAAGGGCCAGCCTC |
| | GGGAACCCCAGGTTTACACATTGCCTCCATCTCGGGACGAGCTGACCAA |
| | GAACCAGGTGTCCCTGACCTGTCTCGTGAAGGGCTTCTACCCCTCCGATA |
| | TCGCCGTGGAATGGGAGTCCAATGGCCAGCCTGAGAACAACTACAAGA |
| | CAACCCCTCCTGTGCTGGACTCCGACGGCTCATTCTTCCTGTACTCCAAG |
| | CTGACAGTGGACAAGTCCAGATGGCAGCAGGGCAACGTGTTCTCCTGCT |
| | CCGTGATGCACGAGGCCCTGCACAATCACTACACCCAGAAGTCCCTGTCT |
| | CTGTCCCCTGGAAAAGGGGGGGGAGGATCTGGCGGAGGTGGAAGCGG |
| | AGGCGGTGGATCTGAAGTTGCAGCTGTTGGAAAGTGGGGGGGGATTGGT |
| | TCAGCCTGGCGGATCTCTGAGACTGTCTTGTGCCGCCTCCGGCTTTACCT |
| | TCTCCTCCTACGCTATGTCCTGGGTCCGACAGGCTCCCGGAAAAGGACTT |
| | GAATGGGTGTCCCAGATCTCCCCTGCTGGGGGCTACACCAATTACGCCG |
| | ACTCTGTGAAGGGCAGATTCACCATCTCTCGGGACAACTCCAAGAACAC |
| | CCTGTACCTGCAGATGAACTCCCTGAGAGCCGAGGACACCGCCGTGTAC |
| | TATTGTGCTAGAGGCGAGCTGCCCTACTTCCGGATGTCCAAAGTGATGG |
| | ACGTGTGGGGACAGGGAACCCTCGTGACAGTTTCTAGTGGTGGCGGAG |
| | GAAGCGGGGGAGGGGGTTCTGGCGGTGGCGGATCTGAAATTGTGCTGA |
| | CCCAGTCTCCAGGCACACTCAGTTTGAGCCCTGGCGAGAGAGCTACCCT |
| | GAGCTGTAGAGCCTCTCAGTTCCTGTCCAGCTACCTGGCTTGGTATCAGC |
| | AGAAGCCAGGACAGGCCCCTCGGCTGTTGATCTATGGCGCTTCTGCTAG |
| | AGCCAGCGGCATCCCTGATAGATTCTCCGGCTCTGGCTCTGGCACCGACT |
| | TCACCCTGACAATCTCCCGGCTGGAACCTGAGGACTTCGCTGTGTACTAC |
| | TGCCAGCAGTACCTGGCCTCTCCTGCCACATTTGGCCAGGGAACAAAGG |
| | TCGAGATCAAGTGA |
| SEQ ID NO: 25 | GACATCCAGATGACCCAGTCTCCATCATCCCTGTCGGCCTCAGTGGGCGA |
| Encodes polypeptide | CAGAGTGACCATCACTTGTCAAGCCTCCCAAGACATTAGCAACTACCTGA |
| SEQ ID NO: 12 | ACTGGTACCAGCAGAAGCCCGGAAAGGCCCCGAAGCTGCTCATCTATGA |
| | CGCTTCCAACCTTGAGACTGGAGTGCCTTCGCGCTTCTCCGGCTCCGGGA |
| | GCGGTACCGATTTCACCTTCACCATCTCCTCCCTGCAACCCGAGGACATT |
| | GCGACTTACTTCTGCCAACATTTCGATCACCTCCCTCTCGCGTTCGGGGG |
| | CGGAACTAAGGTCGAGATTAAGCGGACCGTGGCTGCCCCGTCCGTGTTC |
| | ATCTTCCCGCCGTCCGATGAACAGCTGAAGTCCGGTACCGCATCAGTCGT |
| | GTGCTTGCTGAACAACTTCTACCCCCGGGAAGCCAAGGTCCAGTGGAAA |
| | GTGGACAATGCGCTGCAGTCGGGAAACTCGCAGGAATCCGTGACCGAA |
| | CAGGATTCGAAGGACAGCACATACAGCCTGTCATCCACCCTCACGCTGTC |
| | GAAGGCCGACTACGAGAAGCACAAAGTGTACGCCTGCGAAGTGACCCA |
| | CCAAGGGCTTAGCAGCCCTGTGACCAAGTCCTTCAACCGCGGAGAGTGC |

Additional exemplary bispecific antibody heavy chain polypeptide sequences for use in a protein degrader of the disclosure are set forth in Table 3 and in SEQ ID NO: 1, 2, 3, 4, 5, 6, 7, 8, 9, 10, 11, and 39, and the polynucleotide sequences encoding the bispecific antibody's heavy chain antibody polypeptide sequence are set forth in Table 3 and in SEQ ID NO: 14, 15, 16, 17, 18, 19, 20, 21, 22, 23, 24, and 40, respectively. In some aspects, the bispecific antibody further comprises a light chain polypeptide sequence comprising a variable light chain and a constant light chain, that pairs with the heavy chain polypeptide and is set forth in SEQ ID NO: 12. The polynucleotide sequence that encodes the light chain polypeptide of SEQ ID NO: 12 is set forth in SEQ ID NO: 25. In some aspects, the disclosure provides the polynucleotide sequence used to encode the GGGGS subunit, and these sequences are set forth in Table 3 and in SEQ ID NOS: 26-38.

In some aspects, the protein degrader is a bispecific antibody comprising an anti-EGFR binding arm comprising (or consisting of) a VH amino acid sequence as shown in SEQ ID NO: 69 and a VL amino acid sequence as shown in SEQ ID NO: 70. In some aspects, the bispecific antibody comprises an anti-EGFR binding arm comprising heavy chain CDR1, CDR2 and CDR3 regions comprising (or consisting of) the amino acid sequences shown in SEQ ID NOs: 71, 72 and 73, respectively. In some aspects, the bispecific antibody comprises an anti-EGFR binding arm comprising a heavy chain CDR3 region comprising (or consisting of) the amino acid sequences shown in SEQ ID NO: 73. In some aspects, the bispecific antibody comprises an anti-EGFR binding arm comprising light chain CDR1, CDR2 and CDR3 regions comprising (or consisting of) the amino acid sequences shown in SEQ ID NOs: 74, 75 and 76, respectively. In some aspects, the bispecific antibody comprises an anti-EGFR binding arm comprising a light chain CDR3 region comprising (or consisting of) the amino acid sequences shown in SEQ ID NO: 76. In some embodiments, an anti-EGFR binding arm comprises (or consisting of) one or more sequences at least 90%, 91%, 92%, 93%, 94%, 95%, 96%, 97%, 98%, 99%, 99.5% or 99.8% identical to any of the aforementioned VH, VL, HCDR or LCDR sequences. These sequences are also shown in Table 3.

In some aspects, the protein degrader is a bispecific antibody comprising an anti-NRP1 binding arm comprising (or consisting of) a VH amino acid sequence as shown in SEQ ID NO: 77 and a VL amino acid sequence as shown in SEQ ID NO: 78. In some aspects, the bispecific antibody comprises an anti-NRP1 binding arm comprising heavy chain CDR1, CDR2 and CDR3 regions comprising (or consisting of) the amino acid sequences shown in SEQ ID NOs: 79, 80 and 81, respectively. In some aspects, the bispecific antibody comprises an anti-NRPT binding arm comprising a heavy chain CDR3 region comprising (or consisting of) the amino acid sequences shown in SEQ ID NO: 81. In some aspects, the bispecific antibody comprises an anti-NRP1 binding arm comprising light chain CDR1, CDR2 and CDR3 regions comprising (or consisting of) the amino acid sequences shown in SEQ ID NOs: 82, 83 and 84, respectively. In some aspects, the bispecific antibody comprises an anti-NRP1 binding arm comprising a light chain CDR3 region comprising (or consisting of) the amino acid sequences shown in SEQ ID NO: 84. In some embodiments, an anti-NRP1 binding arm comprises (or consisting of) one or more sequences at least 90%, 91%, 92%, 93%, 94%, 95%, 96%, 97%, 98%, 99%, 99.5% or 99.8% identical to any of the aforementioned VH, VL, HCDR or LCDR sequences. These sequences are also shown in Table 3.

The EGFR degrader may reduce or prevent resistance of a cancer to a tyrosine kinase inhibitor. The anti-EGFR degrader may be, for example, an antibody, a polynucleotide, a small molecule, or a combination thereof. The EGFR degrader may cause degradation of target protein, reduce or prevent resistance of a cancer to a tyrosine kinase inhibitor. Acquired drug resistance to the tyrosine kinase inhibitor may be decreased or prevented following administration of the EGFR degrader. In some embodiments, tyrosine kinase inhibitor resistance may be decreased by at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold. In some embodiments, tyrosine kinase inhibitor resistance is decreased 2-fold to 10-fold. Tyrosine kinase inhibitor activity may be increased by at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold. In some embodiments, tyrosine kinase inhibitor activity is increased by at least 2-fold to at least 10-fold. In some embodiments, administering to the patient having a cancer that is resistant to the therapeutic agent an amount of the target degrader may increase the therapeutic activity of the therapeutic agent by at least 2-fold. In some embodiments, administering to the patient having a cancer that is resistant to the therapeutic agent an amount of the target degrader may increase the therapeutic activity of the therapeutic agent by at least 3-fold. In some embodiments, administering to the patient having a cancer that is resistant to the therapeutic agent an amount of the target degrader may increase the therapeutic activity of the therapeutic agent by at least 4-fold. In some embodiments, administering to the patient having a cancer that is resistant to the therapeutic agent an amount of the target degrader may increase the therapeutic activity of the therapeutic agent by at least 5-fold. In some embodiments, administering to the patient having a cancer that is resistant to the therapeutic agent an amount of the target degrader may increase the therapeutic activity of the therapeutic agent by at least 6-fold. In some embodiments, administering to the patient having a cancer that is resistant to the therapeutic agent an amount of the target degrader may increase the therapeutic activity of the therapeutic agent by at least 7-fold. In some embodiments, administering to the patient having a cancer that is resistant to the therapeutic agent an amount of the target degrader may increase the therapeutic activity of the therapeutic agent by at least 8-fold. In some embodiments, administering to the patient having a cancer that is resistant to the therapeutic agent an amount of the target degrader may increase the therapeutic activity of the therapeutic agent by at least 9-fold. In some embodiments, administering to the patient an amount of the target degrader may increase the therapeutic activity of the therapeutic agent by at least 10-fold.

a. Neuropilin

There are two forms of neuropilins, NRP-1 and NRP-2. Neuropilins are transmembrane glycoproteins consisting of four domains, A (ala2), B (blb2), C (MAM) and cytoplasmic domain. It is known to regulate neurogenesis and angiogenesis by complexing with Plexin receptors/class-3 semaphorin ligands, and Vascular Endothelial Growth Factor (VEGF) receptors/VEGF ligands, respectively. Neuropilins predominantly act as co-receptors as they have a very small cytoplasmic domain and thus rely upon other cell surface receptors to transduce their signals across a cell membrane (Pellet et al., 2008 and Schwarz, 2010).

Recent studies have shown that neuropilins are multifunctional and can partner with a wide variety of transmembrane receptors. Neuropilins are closely associated with numerous signalling pathways including those activated by Epidermal Growth Factor (EGF), Fibroblast Growth Factor (FGF), Hepatocyte Growth Factor (HGF), Insulin-like Growth Factor (IGF), Platelet Derived Growth Factor (PDGF) and Transforming Growth Factor beta (TGF13) (Kofler, 2016 and Roy et al., 2017). Although neuropilins are commonly found at the cell surface, they have also been reported within the mitochondria and nucleus (Issitt et al., 2019 and Mehta et al., 2018). Both Neuropilin family members can also be found in soluble forms created by alternative splicing or by ectodomain shedding from the cell surface (Rossgnol et al., 2000 and Werneberg et al., 2016).

Neuropilins bind to many receptors including receptor tyrosine kinases (example, EGFR), receptor serine/theroine kinases (example, TGFPR), Integrins, GPCRs (G-protein coupling receptors), ion channels but not limited to CXCRs.

The pleiotropic nature of the NRP receptors results in their involvement in cellular processes, such as axon guidance and angiogenesis, the immune response and remyelination (Mecollari et al., 2014). Therefore, dysregulation of NRP activity has been implicated in many pathological conditions, including many types of cancer and cardiovascular disease (Niland et al., 2019; Kofler, 2016; Pellet-Many et al., 2019; and Harma et al., 2020).

The NRP1 binding domain of a protein degrader used in the methods of the disclosure comprises an antibody, or an NRP1-binding fragment thereof. For example, all or a portion of an NRP1 antibody known in the art or an anti-NRP1 antibody provided herein can be used. Non-limiting examples of anti-NRP1 mAb heavy chain polypeptide sequences are set forth in SEQ ID NOs: 41-47, which includes an N-terminal NPR1 binding domain. In some embodiments, the monoclonal antibody heavy chain polypeptide comprises the N-terminal NRP1 binding domain including a variable heavy chain (VH) and a constant heavy chain 1 (CH1), and Fc domain comprising a constant heavy chain 2 (CH2) and constant heavy chain 3 (CH3). In some embodiments, the monoclonal antibody comprises a light chain polypeptide sequence as set forth in any of SEQ ID NOs: 48-54. Thus, the heavy chains of SEQ ID NOs: 41-47 can pair with the light chains of SEQ ID NOs: 48-54, respectively. The polynucleotide sequences that encode the heavy and light chain polypeptides are set forth in SEQ ID NOs: 55-61 and 62-68, respectively. These sequences are also shown in Table 3.

b. EGFR

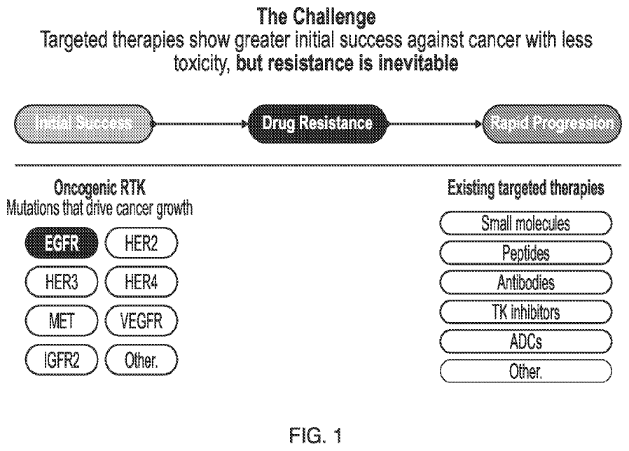

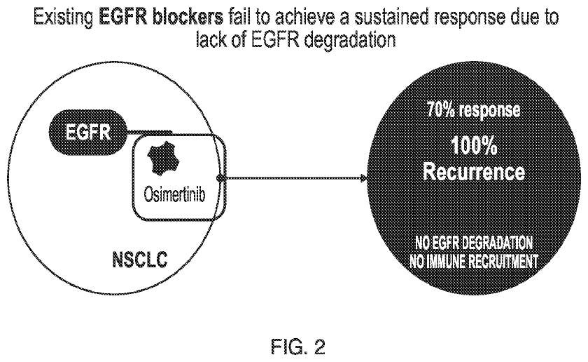

The epidermal growth factor receptor (EGFR) is one of the most frequently altered oncogenes in solid tumors. Increased EGFR signaling drives proliferation and cell survival in many cancer types. Mutations affecting EGFR expression or activity may result in cancer. For example, mutations leading to over-expression of EGFR are associated with the development of a wide variety of cancers. All known EGFR inhibitor as well as all known kinase inhibitors targeting oncogenic receptor tyrosine kinase and serine/threonine kinase have eventually conferred drug resistance (FIG. 1). The existing EGFR blockers fail to achieve a sustained response due to the presence of EGFR due to lack of EGFR degradation (FIG. 2).

c. Additional Protein Degraders
cMET×NRP1 Bispecific Constructs

In an embodiment, the protein degrader is a cMET×NRP1 bispecific construct. In embodiments, a cMET×NRP1 bispecific antibody comprises an anti-NRP1 binding arm comprising heavy chain CDR1, CDR2 and CDR3 regions comprising (or consisting of) the amino acid sequences shown in SEQ ID NOs: 79, 80 and 81, respectively. In an embodiment, the bispecific antibody comprises an anti-NRP1 binding arm comprising a heavy chain CDR3 region comprising (or consisting of) the amino acid sequences shown in SEQ ID NO: 81. In embodiments, the bispecific antibody comprises an anti-NRP1 binding arm comprising light chain CDR1, CDR2 and CDR3 regions comprising (or consisting of) the amino acid sequences shown in SEQ ID NOs: 82, 83 and 84, respectively. In an embodiment, the bispecific antibody comprises an anti-NRP1 binding arm comprising a light chain CDR3 region comprising (or consisting of) the amino acid sequences shown in SEQ ID NO: 84.

In embodiments, the cMET binding arm comprises sequences of an anti-cMET monoclonal antibody (mAb) known and available in the art, e.g., the heavy and light chain CDRs (1-3) of the mAb or the VH/VL polypeptides of the mAb. In an embodiment, the anti-cMET mAb is emibetuzumab (also known in the art as LY2875358). In an embodiment, the anti-cMET mAb is onartuzumab. In an embodiment, the anti-cMET mAb is telisotuzumab.

### HER2×NRP1 Bispecific Construct

In an embodiment, the protein degrader is a HER2×NRP1 bispecific antibody construct. In embodiments, a HER2×NRP1 bispecific antibody comprises an anti-NRP1 binding arm comprising heavy chain CDR1, CDR2 and CDR3 regions comprising (or consisting of) the amino acid sequences shown in SEQ ID NOs: 79, 80 and 81, respectively. In an embodiment, the bispecific antibody comprises an anti-NRP1 binding arm comprising a heavy chain CDR3 region comprising (or consisting of) the amino acid sequences shown in SEQ ID NO: 81. In embodiments, the bispecific antibody comprises an anti-NRP1 binding arm comprising light chain CDR1, CDR2 and CDR3 regions comprising (or consisting of) the amino acid sequences shown in SEQ ID NOs: 82, 83 and 84, respectively. In an embodiment, the bispecific antibody comprises an anti-NRP1 binding arm comprising a light chain CDR3 region comprising (or consisting of) the amino acid sequences shown in SEQ ID NO: 84.

In embodiments, the HER2 binding arm comprises sequences of an anti-HER2 monoclonal antibody (mAb) known and available in the art, e.g., the heavy and light chain CDRs (1-3) of the mAb or the VH/VL polypeptides of the mAb. In an embodiment, the anti-HER2 mAb is trastuzumab. In an embodiment, the anti-HER2 mAb is pertuzumab.

### IGF1R×NRP1 Bispecific Constructs

In an embodiment, the protein degrader is an IGF1R×NRP1 bispecific antibody construct. In embodiments, an IGF1R×NRP1 bispecific antibody comprises an anti-NRP1 binding arm comprising heavy chain CDR1, CDR2 and CDR3 regions comprising (or consisting of) the amino acid sequences shown in SEQ ID NOs: 79, 80 and 81, respectively. In an embodiment, the bispecific antibody comprises an anti-NRP1 binding arm comprising a heavy chain CDR3 region comprising (or consisting of) the amino acid sequences shown in SEQ ID NO: 81. In embodiments, the bispecific antibody comprises an anti-NRP1 binding arm comprising light chain CDR1, CDR2 and CDR3 regions comprising (or consisting of) the amino acid sequences shown in SEQ ID NOs: 82, 83 and 84, respectively. In an embodiment, the bispecific antibody comprises an anti-NRP1 binding arm comprising a light chain CDR3 region comprising (or consisting of) the amino acid sequences shown in SEQ ID NO: 84.

In embodiments, the IGF1R binding arm comprises sequences of an anti-IGF1R monoclonal antibody (mAb) known and available in the art, e.g., the heavy and light chain CDRs (1-3) of the mAb or the VH/VL polypeptides of the mAb. In an embodiment, the anti-IGF1R mAb is ganitumab (also known in the art as AMG 479). In an embodiment, the anti-IGF1R mAb is figitumumab. In an embodiment, the anti-IGF1R mAb is cixutumumab. In an embodiment, the anti-IGF1R mAb is dalotuzumab.

### 3. Therapeutic Agents Including Tyrosine Kinase Inhibitors

a. Tyrosine Kinase

A tyrosine kinase is an enzyme that can transfer a gamma (terminal) phosphate group from ATP to a polypeptide in a cell. The phosphate group is attached to the amino acid tyrosine on the polypeptide. The tyrosine kinase may phosphorylate one or more tyrosine residues on a polypeptide. The phosphorylation of the tyrosine on the polypeptide can cause a change in the function of the polypeptide. Tyrosine kinases are a subgroup of the larger class of protein kinases that attach phosphate groups to other amino acids (such as serine and threonine). Phosphorylation of proteins by kinases is one mechanism in communicating signals within a cell (signal transduction) and regulating cellular activity, such as cell division.

b. Tyrosine Kinase Inhibitor

Tyrosine kinase inhibitor (TKI) inhibits the activity or expression of a tyrosine kinase. A tyrosine kinase inhibitor may include an antibody, a polynucleotide such as interfering RNA, a small molecule, or a combination thereof. In some embodiments, the tyrosine kinase inhibitor comprises an antibody. In some embodiments, the antibody is a monoclonal antibody. In some embodiments, the tyrosine kinase inhibitor comprises a small molecule. The tyrosine kinase inhibitor may be specific for a particular tyrosine kinase, or for a particular family or subfamily of tyrosine kinases. In some embodiments, the tyrosine kinase inhibitor is specific for EGFR. Such inhibitors may be referred to as “EGFR inhibitors.” Antibody EGFR inhibitors include, for example, cetuximab and panitumumab. Small molecule EGFR inhibitors include, for example, lapatinib (a mixed EGFR and ERBB2 inhibitor), mereletinib (osimertinib), gefitinib, icotinib, erlotinib, afatinib, and brigatinib. The EGFR inhibitor may bind and inhibit the kinase domain of the EGFR. Without kinase activity, the EGFR may not be able to activate itself or induce downstream activation and signaling. In some embodiments, the EGFR inhibitor comprises Osimertinib.

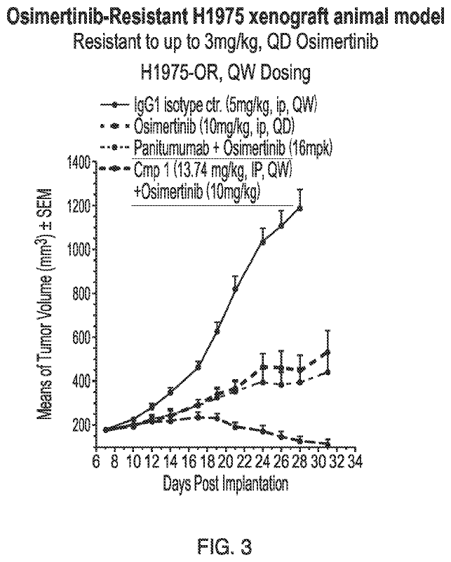

All following and but limited to EGFR inhibitors that target and inhibit certain oncogenic EGFRs and eventually confer drug resistance, which is overcome with the treatment of any EGFR degrader including the present invention (Compound 1 in Example 1 and FIG. 3) alone and in combination with any EGFR inhibitor that causes drug resistance.

EGFR inhibitors are Lazertinib, Osimertinib (AZD9291), WZ4002, Cyasterone, Erlotinib (OSI-774) HCl, efitinib (ZD1839), Lapatinib (GW-572016) Ditosylate, Afatinib (BIBW2992), Saracatinib (AZD0530), Vandetanib (ZD6474), Neratinib (HKI-272), Canertinib (CI-1033), Lapatinib (GW-572016), AG-490 (Tyrphostin B42), CP-724714, Dacomitinib (PF-00299804), Sapitinib (AZD8931), CUDC-101, AG-1478 (Tyrphostin AG-1478), PD153035 HCl, Pelitinib (EKB-569), AC480 (BMS-599626), AEE788 (NVP-AEE788), AP26113-analog (ALK-IN-1), OSI-420, WZ3146, HER2-Inhibitor-1, WZ8040, Allitinib tosylate, Rociletinib (CO-1686), Genistein (NPI 031L), Varlitinib, TQB33804 (EGFR-IN-7), Icotinib (BPI-2009H), TAK-285, Daphnetin, Tyrphostin 9, AG-18, AG 555, AZ5104, CL-387785 (EKI-785), Tyrphostin AG-258, AG-556, Tucatinib, Erlotinib (OSI-774), Gefitinib-based PROTAC 3, Zorifertinib (AZD 3759), ErbB2, AV-412 free base, AST-1306, JND3229, BI-4020, Theliatinib (HMPL-309), BDTX-189, Lifirafenib (BGB-283), Pyrotinib (SHR-1258), O-Demethyl-Gefitinib, Epertinib hydrochloride, SU5214, Avitinib (AC0010), AG 494, and not limited to Poziotinib (HM781-36B).

All following and but limited to ERBB2 (HER2), ERBB3 (HER3) and ERBB4 (HER4) inhibitors target and inhibit certain oncogenic ERBBs and eventually confer drug resistance, which is overcome with the treatment of any ERBB degrader and current invention alone and in combination with any ERBBs inhibitor that causes drug resistance.

ERBB inhibitors are Tucatinib, HER2-inhibitor-1, Afatinib (BIBW2992), Neratinib (HKI-272), CP-724714, Mubritinib (TAK 165), AC480 (BMS-599626), AEE778, TAK-285, Tyrphostin AG 879, Tyrphostin AG-528, SU5204, Poziotinib (HM781-36B). TAS0728, BDTX-189, Pyrotinib, and Epertinib Hydrochloride.

All following and but limited to cMET inhibitors that target and inhibit certain oncogenic cMET s and eventually confer drug resistance, which is overcome with the treatment of any cMET degrader and current invention alone and in combination with any cMET inhibitor that causes drug resistance.

cMET inhibitors are Crizotinib, Cabozantinib, Foretinib, PHA-665752, SU11274, SGX-523, BMS-777607, Tivantinib, JNJ-38877605, PF-04217903, Amuvatinib (MP-470), MGCD-265 analog, Capmatinib, BMS-754807, BMS-794833, AMG-208, MK-2461, Golvatinib, AMG-458, NVP-BVU972, AMG 337, Merestinib, JNJ-38877618, Crizotinib hydrochlorode, Ningetinib, AMG-1, UNC2025, Pamufetinib, Altiratinib, NPS-1304, Savolitinib.

All following and but limited to PDGFR and FGFR inhibitors that target and inhibit certain oncogenic PDGFRs and FGFRs and eventually confer drug resistance, which is overcome with the treatment of any PDGFR degrader and any FGFR degrader and current invention alone and in combination with any PDGFR or any FGFR inhibitor that causes drug resistance.

PDGFR inhibitors and FGFR inhibitors are Ponatinib (AP24534), Infigratinib (BGJ398), Nintedanib (BIBF 1120), Pazopanib HCl (GW786034 HCl), Pazopanib, AZD4547, Tyrphostin AG 1296, SSR128129E, LY2874455, Derazantinib (ARQ-087), SU5402, ODM-203, Pemigatinib (INCB054828), Lucitanib (E3810) hydrochloride, Ferulic Acid, Masitinib mesylate, Fisogatinib (BLU-554), PRN1371, ON123300, FIIN-3, Roblitinib (FGF401), Futibatinib (TAS-120), FIIN-2, Zoligratinib (Debio-1347), Nintedanib Ethanesulfonate Salt, BLU9931, and Sulfatinib.

All following and but limited to cKIT inhibitors that target and inhibit certain oncogenic cKITs and eventually confer drug resistance, which is overcome with the treatment of any cKIT degrader and current invention alone and in combination with each cKIT inhibitor that causes drug resistance. [0092] cKIT inhibitors are Dasatinib (BMS-354825), Sorafenib (BAY 43-9006) tosylate, Imatinib (ST1571) Mesylate, Sunitinib (SU11248) malate, Ponatinib (AP24534), Axitinib (AG 013736), Imatinib (ST1571), Nintedanib (BIBF 1120), Regorafenib (BAY 73-4506), Pazopanib HCl (GW786034 HCl), Linifanib (ABT-869), Crenolanib (CP-868596), Masitinib (AB1010), Anuvatinib (MP-470), Orantinib (SU6668), CP-673451, Telatinib, PP121, Pazopanib, Tyrphostin AG 1296, Tyrphostin 9, SU14813, Regorafenib Hydrochloride, Tyrphostin AG1433, Sunitinib (SU11248), Ripretinib (DCC-2618), Masitinib mesylate, AZD3229, ON123300, Avapritinib (BLU-285), Seralutinib (GB002), AZD2932, JNJ-10198409, Nintedanib Ethanesulfonate Salt, Flumatinib (HI-I-GV-678), and Regorafenib (BAY-734506) Monohydrate.

All following and but limited to FTL3 inhibitors that target and inhibit certain oncogenic FTL3 and eventually confer drug resistance, which is overcome with the treatment of any FTL3 degrader and current invention alone and in combination with any FTL3 inhibitor that causes drug resistance. FTL3 inhibitors are Pacritinib (SB1518), TCS 359, Linifanib (ABT-869), Zotiraciclib, Cediranib (AZD2171), Dovitinib (TK1258) Lactate, UNC2025 HCl, SU5614, FLT3-IN-2, FLT3-IN-4, FF-10101, Merestinib (LY2801653), Emavusertib (CA-4948), Tandutinib (MLN518), R406 (free base), 5′-Fluoroindirubinoxime, Tozasertib, Quizartinib (AC220), R406, AST-487 (NVP-AST487), Sorafenib (BAY 43-9006) tosylate, BMS-794833, Sorafenib (BAY 43-9006), 4SC-203, Dovitinib (TKI-258), Isoguanosine, TAK-659, ATH686, SGI-1776 free base, MK-2461, Fostamatinib (R788) disodium, MRX-2843, Brigatinib (AP26113), GW2580, Rebastinib (DCC-2036), BMS-754807, UNC2025, Fedratinib (TG101348), FLT3-IN-3, G-749, Crenolanib (CP-868596), BPR1K871, PHA-680632, Entospletinib (GS-9973), HPK1-IN-2, SP600125, SKLB4771 (FLT3-IN-1), KW-2449, Gilteritinib (ASP2215), CCT241736, PRT062607 (P505-15) HCl, ENMD-2076, FN-1501, Ceritinib (LDK378), Silmitasertib (CX-4945), AZD2932, Fostamatinib (R788), Tivozanib (AV-951), PF-477736, BPR1J-097, Pexidartinib (PLX3397), Go6976, HM43239, OSI-930, TG101209, PLX5622, Amuvatinib (MP-470), GNF-2, Midostaurin (PKC412), AMG 925, and ENMD-2076 L-(+)-Tartaric acid.

All following and but limited to VEGFR1, VEGFR2, VEGFR3, and VEGFR4 inhibitors that target and inhibit any one of oncogenic VEGFR1, VEGFR2, VEGFR3, and VEGFR4 and eventually confer drug resistance, which is overcome with the treatment of any one of VEGFR1, VEGFR2, VEGFR3, or VEGFR4 degrader and current invention alone and in combination with any of VEGFR1, VEGFR2, VEGFR3, or VEGFR4 inhibitor that causes drug resistance.

VEGFR1, VEGFR2, VEGFR3, and VEGFR4 inhibitors are Sorafenib (BAY 43-9006) tosylate, Sunitinib (SU11248) malate, Lenalidomide (CC-5013), Cabozantinib (BMS-907351), Ponatinib (AP24534), Axitinib (AG 013736), Foretinib (GSK1363089), Vandetanib (ZD6474), Nintedanib (BIBF 1120), Regorafenib (BAY 73-4506), Pazopanib HCl (GW786034 HCl), Cediranib (AZD2171), PD173074, Dovitinib (TKI-258), Linifanib (ABT-869), Vatalanib (PTK787) 2HC1, RAF265 (CHIR-265), Tivozanib (AV-951), Motesanib Diphosphate (AMG-706), Lenvatinib (E7080), Brivanib (BMS-540215), MGCD-265 analog, AEE788 (NVP-AEE788), ENMD-2076, OSI-930, CYC116, Ki8751, Telatinib, PP121, Pazopanib, KRN 633, SAR131675, BMS-794833, Apatinib (YN968D1) mesylate, Sorafenib (BAY 43-9006), Cabozantinib malate, Brivanib Alaninate (BMS-582664), Golvatinib (E7050), Semaxanib (SU5416), ZM 306416, ZM 323881 HCl, ENMD-2076 L-(+)-Tartaric acid, LY2874455, BAW2881 (NVP-BAW2881), WHI-P180, SU14813, ZD-4190, SU1498, SU5402, PDGFR inhibitor 1, Ki20227, Dovitinib (TKI258) Lactate, Toceranib phosphate, Cediranib Maleate, Apatinib, BFH772, Lenvatinib (E7080) Mesylate, SU5614, Regorafenib Hydrochloride, SU5204, SU5208, Fruquintinib (HMPL-013), hVEGF-IN-1, ODM-203, Erdafitinib (JNJ-42756493), Tyrphostin AG1433, MAZ51, SKLB 610, Sunitinib (SU11248), 4SC-203, Sitravatinib (MGCD516), R1530, Donafenib (Sorafenib D3), Emvododstat (PTC299), AG-13958, SKLB1002, Motesanib (AMG-706), 4,4′-Bis(4-aminophenoxy)biphenyl, Lucitanib (E3810) hydrochloride, Oglufanide, Chiauranih, Ningetinib, X-82 (Vorolanib), Pamufetinib (TAS-115), Cassia seed Extract, CS-2660 (JNJ-38158471), WAY-340935, (20R)-Protopanaxadiol, Semen litchi Extract, Altiratinib, Vitamin E, SU5408, AZD2932, Anlotinib (AL3818) dihydrochloride, Nintedanib Ethanesulfonate Salt, Chebulinic acid, SU5205, SU5214, Regorafenib (BAY-734506) Monohydrate, Taxifolin (Dihydroquercetin), and Sulfatinib, and XL092.

All following and but limited to TGFβ1R and TGFβ2R inhibitors that target and inhibit certain oncogenic TGFβ1R and TGFβ2R and eventually confer drug resistance, which is overcome with the treatment of any TGFP1R and/or TGFβ2R degrader and current invention alone and in combination with any of TGFβ1R and TGFβ2R inhibitor that causes drug resistance.

TGFβ1R and TGFβ2R inhibitors are SD-208, GW788388, A-83-01, Disitertide (P144), SRI-011381, TP0427736 HCl, LY2109761, Ophiopogonin D, SB505124, SIS3 HCl, BIBF-0775, LY 3200882, LSKL, Inhibitor of Thrombospondin (TSP-1), Galunisertib (LY2157299), Ginsenoside Rh4, LDN-193189, A77-01, LDN-193189 2HC1, Vactosertib (TEW-7197), Halofuginone hydrobromide, Halofuginone, Sulfasalazine (NSC 667219), BMS-986260, XAV-939, LY364947, Oxymatrine, Pirfenidone (5-7701), Hypaconitine, SB525334, ITD-1, Gamabufotalin, TA-02, and PD 169316.

All following and but limited to Integrins inhibitors target and inhibit certain oncogenic Integrins and eventually confer drug resistance, which is overcome with the treatment of any Integrins degrader and current invention alone and in combination with any Integrin inhibitor that causes drug resistance.

Integrins inhibitors are Cilengitide trifluoroacetate, RGD (Arg-Gly-Asp) Peptides, A-205804, SB273005, Cilengitide, RGD peptide (GRGDNP), OSU-T315, ILK-IN-3, Cyclo (-RGDfK), A286982, Cyclo(RGDyK), and A286982.

All following and but limited to IGF1R and JR inhibitors that target and inhibit certain oncogenic IGF1R and IR and eventually confer drug resistance, which is overcome with the treatment of any IGF1R and IR degrader and current invention alone and in combination with any of IGF1R or IR inhibitor that causes drug resistance.

IGF1R and IR inhibitors are Luminespib (NVP-AUY922), Linsitinib (OSI-906), NVP-AEW541, GSK1904529A, NVP-ADW742, BMS-536924, Ceritinib (LDK378), AG-1024, GSK1838705A, BMS-754807, PQ 401, ZD3463, Nordihydroguaiaretic acid (NDGA), NT157, Insulin (human), Ceritinib dihydrochloride, Dioscoreae Nipponicae Rhizoma Extract, MID-1, Brigatinib (AP26113), Picropodophyllin (PPP), MSDC-0160, Insulin Degludec, Chromium picolinate, SBI-477, and XL228.

### 4. Additional Target Proteins

Neuropilins are closely associated with many drug-resistant tumors. Neuropilins and their degradation are associated with receptor tyrosine kinases, receptor serine/threonine kinases, G-Protein coupled receptors, Ion-channel, CXCR, and immune checkpoint modulators for many biological functions. Therefore, the co-degradation of neuropilin and other receptors is useful to treat drug-resistance associated with neuropilins.

All following and but limited to PARP inhibitors that confer drug resistance, which is overcome with the treatment of any NRP degradation compound alone and in combination with any PARP1 inhibitor that causes drug resistance.

PARP inhibitors are PJ34 HCl, AZD2461, Olaparib (AZD2281), Veliparib (ABT-888), XAV-939, Rucaparib (AG-014699) phosphate, Iniparib (BSI-201), Talazoparib (BMN 673), AG-14361, 3-Aminobenzamide, A-966492, Niraparib (MK-4827), UPF 1069, ME0328, Licochalcone D, DR2313, MN 64, 4′,5,7-Trimethoxyflavone, Rucaparib, M2912, GeA-69, BYK204165, BGP-15 2HC1, Atamparib (RBN-2397), Venadaparib (IDX-1197), Niraparib (MK-4827) tosylate, NU1025, Rucaparib Camsylate, Berberine chloride (NSC 646666), Pamiparib (BGB-290), Fluzoparib (SHR-3162), G007-LK, NVP-TNKS656, Berberine chloride hydrate, HI-TOPK-032, Stenoparib (E7449), 4-Hydroxyquinazoline, NMS-P118, WIKI4, RBN012759, AZD5305, AZD-9574, RK-287107, Benzamide, JW55, and Picolinamide.

All following and but limited to RAF inhibitors that confer drug resistance, which is overcome with the treatment of any neuropilin degradation compound alone and in combination with any RAF inhibitor that causes drug resistance.

RAF inhibitors are Vemurafenib (PLX4032), B-Raf inhibitor 1 (Compound 13) dihydrochloride, Raf inhibitor 1, Raf inhibitor 2, Sorafenib (BAY 43-9006) tosylate, PLX-4720, Dabrafenib (GSK2118436), Regorafenib (BAY 73-4506), Doramapimod (BIRB 796), GDC-0879, RAF265 (CHIR-265), AZ 628, NVP-BHG712, SB590885, ZM 336372, Sorafenib (BAY 43-9006), GW5074, TAK-632, Agerafenib (RXDX-105), Encorafenib (LGX818), BAW2881 (NVP-BAW2881), PLX8394, TBAP-001, Regorafenib Hydrochloride, MCP110, Naporafenib (LXH254), B-Raf IN 1, Donafenib (Sorafenib D3), CCT196969, RAF709, Lifirafenib (BGB-283), L-779450, PLX7904, LY3009120, Dabrafenib Mesylate, R05126766 (CH5126766), AZ304, Belvarafenib (HM95573), Regorafenib (BAY-734506) Monohydrate, and Tovorafenib (MLN2480).

All following and but limited to autophagy activator or autophagy inhibitors that confer drug resistance, which is overcome with the treatment of any neuropilin degradation compound alone and in combination with any autophagy activator or any inhibitor that causes drug resistance.

Autophagy activator are Enzalutamide (MDV3100), Obatoclax Mesylate (GX15-070), SRT1720 HCl, Fulvestrant (ICI-182780), Bicalutamide (ICI-176334), Resveratrol (SRT501), Colforsin, Rosiglitazone (BRL-49653) maleate, Rosiglitazone (BRL 49653), Mifepristone (RU486), Purmorphamine, Clemastine (HS-592) fumarate, GW4064, Chloroquine diphosphate, Ivermectin (MK-933), Loperamide HCl, Melatonin (NSC 113928), Methylprednisolone (NSC-19987), Clonidine HCl, Flubendazole, Fenofibrate (NSC-281319), Montelukast Sodium, LYN-1604, EN6, Eprenetapopt (APR-246), 3BDO, MHY1485, Methylprednisolone Acetate, QX77, Anisomycin, I3-Elemene, Spermidine trihydrochloride, Troglitazone (CS-045), Corynoxine, Obeticholic Acid, SMER28, BC1618, Monomethyl Fumarate, PCNA-Il, CA77.1, Spermidine, Xylitol, Isorhychophylline, MPP+ iodide.

Autophagy inhibitors are MK-2206 2HC1, Bortezomib (PS-341), Olaparib (AZD2281), Vemurafenib (PLX4032), Vorinostat (SAHA), ABT-737, Y-27632 2HC1, Dactolisib (BEZ235), Sorafenib (BAY 43-9006) tosylate, Dasatinib (BMS-354825), Rapamycin (AY-22989), Crizotinib (PF-02341066), Erlotinib (OSI-774) HCl, Everolimus (RAD001), Gefitinib (ZD1839), Veliparib (ABT-888), Entinostat (MS-275), BI 2536, Pictilisib (GDC-0941), Laduviglusib (CHIR-99021) HCl, LY294002, Trametinib (GSK1120212), Ruxolitinib (INCB018424), Panobinostat (LBH589), Imatinib (STI571) Mesylate, KU-55933 (ATM Kinase Inhibitor), Alisertib (MLN8237), Afatinib (BIBW2992), U0126-EtOH, Idelalisib, Tozasertib, AZD8055, Saracatinib (AZD0530), Paclitaxel (NSC 125973), SP600125, Ponatinib (AP24534), Tanespimycin (17-AAG), YM155 (Sepantronium Bromide), DAPT (GSI-IX), Cisplatin (NSC 119875), Imatinib (STI571), Nilotinib (AMN-107), Temsirolimus (CCI-779), PI-103, Luminespib (NVP-AUY922), Vandetanib (ZD6474), Gemcitabine (LY-188011) HCl, Mocetinostat (MGCD0103), Regorafenib (BAY 73-4506), SRT1720 HCl, Pazopanib HCl (GW786034 HCl), Bosutinib (SKI-606), Cediranib (AZD2171), Belinostat (PXD101), GSK690693, SB202190 (FHPI), Carfilzomib (PR-171), Fulvestrant (ICI-182780), SB216763, SU11274, Linifanib (ABT-869), Pemetrexed (LY-231514) disodium, Torkinib (PP242), Etoposide (VP-16), Wortmannin (KY 12420), LY2109761, Danusertib (PHA-739358), Silmitasertib (CX-4945), Venetoclax (ABT-199), Flavopiridol (L86-8275), SGI-1776 free base, Lapatinib (GW-572016), Vincristine (NSC-67574) sulfate, Temozolomide (CCRG 81045), Tacrolimus (FK506), Metformin HCl, Fasudil (HA-1077) HCl, Oxaliplatin (NSC 266046), Rabusertib (LY2603618), 3-Methyladenine (3-MA), Flavopiridol (L86-8275) HCl, (+)-JQ1, Zoledronic acid (ZOL 446), Momelotinib (CYT387), Ixazomib Citrate (MLN9708) Analogue, Tamoxifen (ICI 46474) Citrate, Letrozole (CGS 20267), Topotecan (NSC609699) HCl, Torin 1, Omipalisib (GSK2126458), Degrasyn (WP1130), 2-Methoxyestradiol (2-MeOE2), Azacitidine (5-Azacytidine), BX-795, OSI-027, Ixazomib (MLN2238), TWS119, Apitolisib (GDC-0980), BI-D1870, Resveratrol (SRT501), Dexamethasone (MK-125), Cytarabine (U-19920A), Simvastatin (MK 733), Vistusertib (AZD2014), CCT128930, Idarubicin HCl, Gemcitabine (LY-188011), Verteporfin (CL 318952), PF-4708671, Torin 2, Streptozotocin (STZ), H 89 2HC1, Brefeldin A, Geldanamycin (NSC 122750), MK-5108 (VX-689), Colforsin, Valproic Acid (NSC 93819) sodium salt, Pitavastatin (NK-104) calcium, Dorsomorphin (Compound C) 2HC1, Lovastatin (MK-803), Rosiglitazone (BRL-49653) maleate, Necrostatin-1, Pazopanib, Nocodazole (R17934), Daporinad, Clofarabine, Cabazitaxel (XRP6258), Atorvastatin Calcium, (R)-(−)-Gossypol acetic acid, Pifithrin-a (PFTa) HBr, SN-38, GSK343, BIX 01294, Nutlin-3a, Tigecycline (GAR-936), STF-62247, Binimetinib (MEK162), Itraconazole (R 51211), Milciclib (PHA-848125), Sorafenib (BAY 43-9006), 10058-F4, YM201636, C646, Hydroxyurea (NSC-32065), Bardoxolone Methyl, Sodium butyrate, PR-619, Linagliptin (BI-1356), Niclosamide (BAY2353), Nitazoxanide (NSC 697855), UNC1999, Heparin sodium, Dynasore, Curcumin, Celastrol (NSC 70931), GSK2606414, Honokiol (NSC 293100), Laduviglusib (CHIR-99021), GANT61, Omeprazole, Amiodarone (NSC 85442) HCl, Dexamethasone Sodium Phosphate, Aspirin (NSC 27223), AZD3463, GSK2656157, Carbamazepine, Bafilomycin Al (Baf-A1), Azithromycin (CP-62993), Nimodipine, IU1, Sulfasalazine (NSC 667219), Pifithrin-μ, PF-543 hydrochloride, Tubastatin A, Nitrendipine, DC661, KB-R7943 mesylate, Tubastatin A TFA, PFK15, Nordihydroguaiaretic acid (NDGA), Nilotinib hydrochloride monohydrate, Salirasib, Neferine, Emetine hydrochloride, SBI-0206965, Apatinib, EAD1, Lanatoside C, Ruxolitinib Phosphate, Losmapimod (GW856553X), STO-609, IITZ-01, Vacuolin-1, Entrectinib (RXDX-101), Sunitinib (SU11248), Valproic acid (VPA), URMC-099, Spautin-1, Erlotinib (OSI-774), Pemetrexed Disodium Hydrate, Crizotinib hydrochloride, Atorvastatin, Berberine chloride (NSC 646666), Quercetin (NSC 9221), VLX600, Afatinib (BIBW2992) Dimaleate, FL-411, Pemetrexed, Autophinib, Cryptotanshinone, Nortriptyline hydrochloride, Dihydroartemisinin (DHA), KN-93 Phosphate, Telaglenastat (CB-839), Purvalanol A, Brevilin A, Chloroquine (NSC-187208), PFK158, Sulfacetamide sodium salt hydrate, OTS964, Sinomenine hydrochloride, MRT67307 HCl, Lys05, Sulfacetamide Sodium, Resatorvid (TAK-242), DMH1, Hydroxychloroquine Sulfate (NSC 4375), MRT68921 HCl, PHY34, Vinorelbine ditartrate (KW-2307), CA-5f, VPS34-IN1, DMOG, ICCB-19 hydrochloride, AS1842856, SAR405, PIK-III, ULK-101, Vinblastine (NSC-49842) sulfate, SP2509, ROC-325, Codonopsis Pilosula Extract, ABTL-0812, ML-9 HCl, AZD1208, Lucanthone, Leonurine, VER155008, RA-190, LY3009120, 4-Phenylbutyric acid (4-PBA), NSC 185058, E260, Daurisoline, Dorsomorphin (Compound C), Tamoxifen (ICI 46474), Deferoxamine mesylate (Ba 33112), Pepstatin A, Trametinib DMSO solvate, and Concanavalin A.

All following and but limited to mTOR inhibitors that confer drug resistance, which is overcome with the treatment of any neuropilin degradation compound alone and in combination with any mTOR inhibitor that causes drug resistance.

mTOR inhibitors are mTOR inhibitor-1, Everolimus (RAD001), KU-0063794, Dactolisib (BEZ235), Rapamycin (AY-22989), AZD8055, Temsirolimus (CCI-779), PI-103, NU7441 (KU-57788), Torkinib (PP242), Ridaforolimus (Deforolimus, MK-8669), Sapanisertib (MLN0128), Voxtalisib (XL765) Analogue, Torin 1, Omipalisib (GSK2126458), OSI-027, PF-04691502, Apitolisib (GDC-0980), GSK1059615, Gedatolisib (PKI-587), WYE-354, Vistusertib (AZD2014), Torin 2, WYE-125132 (WYE-132), BGT226 (NVP-BGT226) maleate, Palomid 529 (P529), PP121, WYE-687, Nitazoxanide (NSC 697855), WAY-600, ETP-46464, GDC-0349, XL388, 4EGI-1. JR-AB2-011, Rotundic acid, Lanatoside C, Compound 401, Astragaloside IV, Ginkgolide K, CC-115, Zotarolimus (ABT-578), Paxalisib (GDC-0084), CZ415, SF2523, Bimiralisib (PQR309), Voxtalisib (XL765), Chrysophanic Acid, Onatasertib (CC 223), 3-Hydroxyanthranilic acid, Samotolisib (LY3023414), MTI-31, ABTL-0812, PQR620, MHY-1685, GNE-477, and GNE-493.

All following and but limited to PI3K activator or PIK3 inhibitors that confer drug resistance, which is overcome with the treatment of any neuropilin degradation compound alone and in combination with any PI3K activator or any PI3K inhibitor that causes drug resistance.

PI3K activators are Demethyl-Coclaurine, Cinobufagin, Resibufogenin, 740 Y-P (PDGFR 740Y-P), Erucic acid, Amarogentin, and YS-49.

PI3K inhibitors are PI-103, XL147 analogue, 3-Methyladenine (3-MA), Apitolisib (GDC-0980), TG100713, Taselisib (GDC 0032), Dactolisib (BEZ235), Pictilisib (GDC-0941), LY294002, Idelalisib, Buparlisib (BKM120), NU7441 (KU-57788), TGX-221, IC-87114, Wortmannin (KY 12420), ZSTK474, Alpelisib (BYL719), AS-605240, PIK-75 HCl, Rigosertib (ON-01910), A66, Voxtalisib (XL765) Analogue, Omipalisib (GSK2126458), PIK-90, AZD6482, PF-04691502, GSK1059615, Duvelisib (IPI-145), Gedatolisib (PKI-587), TG100-115, AS-252424, NU7026, BGT226 (NVP-BGT226) maleate, Fimepinostat (CUDC-907), PIK-294, AS-604850, GSK2636771, Copanlisib (BAY 80-6946), YM201636, CH5132799, CAY10505, PIK-293, PKI-402, VS-5584 (SB2343), KU-0060648, CZC24832, Demethyl-Coclaurine, Oroxin B, Homosalate, AMG319, Cinobufagin, Resibufogenin, Hispidulin, GSK2292767, Lanatoside C, Zeaxanthin, Disitertide (P144), Cafestol, Paxalisib (GDC-0084), SKI-V, MTX-211, Seletalisib (UCB-5857), Trichosanthis Pericarpium Extract, Dioscoreae Nipponicae Rhizoma Extract, GDC-0326, 740 Y-P (PDGFR 740Y-P), PIK-108, Parsaclisib (INCB050465) Hydrochloride, HS-173, SF2523, leniolisib (CDZ 173), Lupenone, Serabelisib (TAK-117), Eganelisib (IP1-549), Quercetin (NSC 9221), Bimiralisib (PQR309), VPS34 inhibitor 1 (Compound 19), IHMT-PI3K6-372, Voxtalisib (XL765), Autophinib, GNE-317, (E)-Akt inhibitor-IV, Notoginsenoside R1, Tenalisib (RP6530), Solasodine, Gallein, Pectolinarin, Inavolisib (GDC-0077), SRX3207, a-Linolenic acid, umbralisib (TGR-1202), acalisib (GS-9820), ME-401, 3-Hydroxyanthranilic acid, Neniralisib, Samotolisib (LY3023414), VPS34-IN1, Ailanthone, Tripterygium wilfordii Extract, IPI-3063, SAR405, PIK-III, IPI-3063, Parsaclisib (INCB050465), Quercetin Dihydrate, Pilaralisib (XL147), SPP-86, AZD8835, Trigonelline, Deguelin, Selective PI3K6 Inhibitor 1 (compound 7n), Loureirin A, PF-4989216, AZD8186, GNE-477, and GNE-493.

All following and but limited to proteasome inhibitors that confer drug resistance, which is overcome with the treatment of any neuropilin degradation compound alone and in combination with any proteasome inhibitor that causes drug resistance.

Proteasome inhibitors are Salinosporamide A (NPI-0052). Bortezomib (PS-341), MG132, Carfilzomih (PR-171), Ixazomib Citrate (MLN9708), Ixazomib (MLN2238), ONX-0914 (PR-957), Oprozomib (ONX 0912), Delanzomib (CEP-18770), Celastrol (NSC 70931), Epoxomicin (BU-4061T), Shikonin (C.I. 75535), VR23, Isoginkgetin, RA-190, and PI-1840.

All following and but limited to JNK inhibitors that confer drug resistance, which is overcome with the treatment of any neuropilin degradation compound alone and in combination with any JNK inhibitor that causes drug resistance.

JNK inhibitors are JNK Inhibitor VIII, JNK Inhibitor IX, JNK-IN-8, BI-78D3, SP600125, Doramapimod (BIRB 796), Metformin HCl, DB07268, 3′-Hydroxypterostilbene, KB-R7943 mesylate, JNK-IN-7, Lourcirin B, Astragaloside IV, Cucurbitacin Iib, Trans-Zeatin, Ezatiostat, IQ 3, Berberine chloride (NSC 646666), SU3327, Bentamapimod (AS602801), Indirubin-3′-oxime, Falcarindiol, Tanzisertib (CC-930), NDMC101, Mulberroside A, C-401 Hydrochloride, RPI-1, Ginsenoside Re, IQ-1S, and Urolithin B.

All following and but limited to NF-κB inhibitors that confer drug resistance, which is overcome with the treatment of any neuropilin degradation compound alone and in combination with any NF-κB inhibitor that causes drug resistance.

NF-κB inhibitors are Phospho-IKB alpha (S32) Rabbit Recombinant mAb, Ophiopogonin D, Cornuside, Rubiadin 1-methyl ether, SM-7368, NF-κB-IN-1, Chitosan oligosaccharide, Cynanchi Atrati Extract, IAXO-102, Adjudin, CBL0137, IQ 3, Hypaphorine, Isoliquiritin apioside, Jaceosidin, Bortezomib (PS-341), Urolithin B, Sulfasalazine (NSC 667219), Acetylcysteine (N-acetylcysteine), Erdosteine, Curcumin, Andrographolide, Dihydroartemisinin (DHA), Indole-3-carbinol, Magnolol, (−)-Parthenolide, Evodiamine, Chondroitin sulfate, TPCA-1, Sodium salicylate, Methylthiouracil, Articaine HCl, L-Quebrachitol, UCB-9260, Zeaxanthin, Tectochrysin, Myrislignan, Gardenoside, Caulophylline (N-Methylcytisine), Demethyleneberberine, 3-Hydroxyanthranilic acid, (E/Z)-IT-603, Triptolide (PG490), Sarsasapogenin, Berbamine dihydrochloride, Pyrrolidinedithiocarbamate ammonium, Stachydrine hydrochloride, Sodium Aescinate, Stachydrine, Tyrosol, Mangiferin, Scutellarin, Ginsenoside Re, Dehydroevodiamine, (E)-Cardamonin, Muscone, Guaiacol, Curcumenol, Schisantherin A, Hederagenin, Astragaloside IV, Ginsenoside Rg 1, Ginsenoside Rbl, Ginsenoside Rd, 4′-Methoxyresveratrol, (+)-a-Lipoic acid, Sodium 4-Aminosalicylate, IMM-H007, Diethylmaleate, 4-Hydroxychalcone, Benfotiamine, QNZ (EVP4593), 4′-Hydroxychalcone, Neferine, Vanillic acid, Hyperoside, Chelidonic acid, Ethyl caffeate, Sulforaphane, (R)-(−)-Ibuprofen, SN50, C25-140, INH14, Licochalcone D, SC75741, JSH-23, Caffeic Acid Phenethyl Ester, Rocaglamide, APX-3330, DTP3, Omaveloxolone (RTA-408), Bardoxolone Methyl, Shikonin (C.I. 75535), TAK-243 (MLN7243), CBL0137 HCl, Withaferin A, NIK SMI1, Alobresib (GS-5829), anthatin, Maslinic acid, Mulberroside A, Eleutheroside E, Berbamine, Engeletin, Aristolochic acid A, Ginsenoside Rb3, 8-O-acetyl shanzhiside methyl ester, Dauricine, 2′,5′-Dihydroxyacetophenone, (+)-Praeruptorin A, Homoplantaginin, Madecassic acid, and BTYNB.

All following and but limited to HSP90 inhibitors that confer drug resistance, which is overcome with the treatment of any neuropilin degradation compound alone and in combination with any HSP90 inhibitor that causes drug resistance.

HSP90 inhibitors are Luminespib (NVP-AUY922), Tanespimycin (17-AAG), Alvespimycin (17-DMAG) HCl, Ganetespib (STA-9090), Elesclomol (STA-4783), B11B021, Tamoxifen (ICI 46474) Citrate, Onalespib (AT13387), NVP-BEP800, Geldanamycin (NSC 122750), SNX-2112 (PF-04928473), PF-04929113 (SNX-5422), KW-2478, XL888, Pifithrin-μ, NMS-E973, Zelavespib (PU-H71), Teprcnone, Dimethylenastron, Apoptozole, KRIBB11, Pseudolaric Acid A, TRC051384, DTHIB, Rocaglamide, KNK437, VER-49009, HA15, Pimitespib (TAS-116), CH5138303, VER-50589, YUM70, Triptolide (PG490), VER155008, JG98, Tamoxifen (ICI 46474), NPX800, and HSP990 (NVP-HSP990).

All following and but limited to E3 ligase inhibitors that confer drug resistance, which is overcome with the treatment of any neuropilin degradation compound alone and in combination with any E3 ligase inhibitor that causes drug resistance.

E3 ligase inhibitors are Lenalidomide (CC-5013), Pomalidomide (CC-4047), Thalidomide (K17), NSC 207895, TAME, PRT4165, CC-885, dCBP-1, Iberdomide (CC220), BC-1215, CC-90009, Avadomide (CC-122), VL285, (S,R,S)-AHPC (MDK7526), Smurfl-IN-A01, (S,R,S)-AHPC-PEG4-NH2 hydrochloride, SZL P1-41, VH298, Thalidomide-OH, MuRF1-IN-1, Skp2 inhibitor Cl (SKPin C1), GMB-475, Mezigdomide (CC-92480), Homo-PROTAC cereblon degrader 1, THAL-SNS-032, NSC232003, Apcin, and Thalidomide-O—COOH (Cereblon ligand 3).

All following and but limited to KRAS inhibitors that confer drug resistance, which is overcome with the treatment of any neuropilin degradation compound alone and in combination with any KRAS ligase inhibitor that causes drug resistance.

KRAS inhibitors are MRTX1133, Sotorasib (AMG510), Adagrasib (MRTX849), LC-2, Deltarasin, BAY-293, ARS-1620, BI-2852, ARS-853 (ARS853), BI-3406, ASP2453, Sotorasib (AMG510) racemate, Pan-RAS-IN-1, MRTX-1257, Zoledronic acid (ZOL 446), Lonafarnib (SCH66336), K-Ras (G12C) inhibitor 9, Salirasib, Alamandine, (Rac)-Antineoplaston A10, K-Ras-1N-1, MCP110, 6H05, K-Ras (G12C) inhibitor 12, Kobe0065, K-Ras (G12C) inhibitor 6, BQU57, Kobe2602, NAV-2729, Antineoplaston A10, Fendiline hydrochloride, KRpep-2d, Pei-illy′ alcohol, RBC8, KY1220, CID-1067700, and Zoledronic acid monohydrate.

### 5. Pharmaceutical Composition

The polypeptides described herein can be formulated into pharmaceutical compositions that further comprise a pharmaceutically acceptable carrier, diluent, adjuvant, or vehicle. In one embodiment, the present disclosure provides a pharmaceutical composition comprising the disclosed polypeptides, and a pharmaceutically acceptable carrier, diluent, adjuvant, or vehicle. In one embodiment, the present invention is a pharmaceutical composition comprising an effective amount of the disclosed bispecific antibodies or a pharmaceutically.

A pharmaceutically acceptable carrier or excipient may contain inert ingredients that do not unduly inhibit the biological activity of the polypeptides. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the polypeptides described herein, such as by producing any undesirable biological effect or otherwise interacting in a deleterious manner with any other component(s) of the pharmaceutically acceptable composition, its use is contemplated to be within the scope of this invention. As used herein, the phrase “side effects” encompasses unwanted and adverse effects of the therapy.

Materials which can serve as pharmaceutically acceptable carriers for antibodies increase conformation stability, reduce protein dynamics, inhibit aggregation, and protect protein adsorbing to liquid air interface, and include but are not limited to cyclodextrin hydrogels, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In some embodiments, a composition of the present invention comprises a pharmaceutically acceptable salt. When polypeptides of the present invention contain relatively acidic functionalities, base addition salts can be obtained by contacting the neutral form of such polypeptides with sufficient amount of the desired base, either neat or in a suitable inert solvent. Examples of pharmaceutically acceptable base addition salts include sodium, potassium, calcium, ammonium, organic amino, or magnesium salt, or a similar salt. When polypeptides of the present invention contain relatively basic functionalities, acid addition salts can be obtained by contacting the neutral form of such polypeptides with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see Berge et al., “Pharmaceutical Salts”, Journal of Pharmaceutical Science, 1977, 66, 1-19). Certain specific polypeptides of the disclosure contain both basic and acidic functionalities that allow the polypeptides to be converted into either base or acid addition salts.

Thus, the disclosed polypeptides may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the polypeptides are preferably regenerated by contacting the salt with a base or acid and isolating the parent polypeptides in the conventional manner. The parent form of the polypeptide may differ from the various salt forms in certain physical properties, such as solubility in polar solvents.

Certain polypeptides of the present invention can exist in unsolvated forms as well as solvated forms, including hydrated forms. In general, the solvated forms are equivalent to unsolvated forms and are encompassed within the scope of the present invention. Certain polypeptides of the present invention may exist in multiple crystalline or amorphous forms. In general, all physical forms are equivalent for the uses contemplated by the present invention and are intended to be within the scope of the present invention.

In some embodiments, subcutaneous formulations may contain recombinant human P1H2O hyaluronidase (rHuPH20) to facilitate dispersion of the antibody from the injection site.

### 6. Combination Therapy

In some embodiments, the compositions and methods detailed herein may be used in combination with other cancer therapies. These additional cancer therapies may be administered to a subject at the same time, before, or after the anti-resistance agent or tyrosine kinase inhibitor. Additional cancer therapies may include, for example, chemotherapy, radiation, and surgery.

Chemotherapy uses one or more chemotherapeutic agents to kill highly proliferating cells. A chemotherapeutic agent includes a compound or composition that is administered in the treatment of cancer or other hyperproliferative disease. Chemotherapeutic agents may be categorized by their mode of activity within a cell, for example, whether and at what stage they affect the cell cycle. Alternatively, a chemotherapeutic agent may be characterized based on its ability to directly cross-link DNA, to intercalate into DNA, or to induce chromosomal and mitotic aberrations by affecting nucleic acid synthesis. Categories of chemotherapeutic agents include, for example, alkylating agents, antimetabolites, antitumor antibiotics, mitotic inhibitors, and nitrosoureas. Specific chemotherapeutic agents may include, for example, cyclophosphamide, doxorubicin, daunorubicin, vinblastine, vincristine, bleomycin, etoposide, topotecan, irinotecan, taxotere, taxol, 5-fluorouracil, methotrexate, gemcitabine, cisplatin, carboplatin, and chlorambucil, and an agonist of any of the above compounds.

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, normal cells are able to repair and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Immunotherapy may include monoclonal antibodies that recognize cancer cells and target them for destruction by the immune system. Such antibodies include rituximah (targeting CD-20), trastuzumah (targeting HER-2), and cetuximab (targeting EGFR). In some embodiments, anticancer antibody is selected from rituximab, trastuzumab, and cetuximab.

Surgery may include full or partial removal of a tumor or cancerous tissue. Surgery may also include removal of any surrounding tissue. Surgery may include removal of any tissue anticipated to be at risk of cancer, such as from spreading or metastasis of cancer to the tissue.

### 7. Administration

The compositions of the present invention may be administered to a subject in need of cancer treatment. The terms “administration” or “administering” refer to the act of providing a composition of the present invention, e.g., a polypeptide or pharmaceutically acceptable salt thereof, to a subject in need of cancer treatment.

As used herein, “intermittent administration” includes the administration of an agent for a period of time (which can be considered a “first period of administration”), followed by a time during which the composition is not taken or is taken at a lower maintenance dose (which can be considered “off-period”) followed by a period during which the composition is administered again (which can be considered a “second period of administration”). Generally, during the second phase of administration, the dosage level of the agent will match that administered during the first period of administration but can be increased or decreased as medically necessary.

The anti-resistance agents and/or tyrosine kinase inhibitors as detailed herein, or the pharmaceutical compositions comprising the same, may be administered to a subject. The anti-resistance agents and/or tyrosine kinase inhibitors as detailed herein can be formulated into a composition and administered in dosages and by techniques well known to those skilled in the medical arts taking into consideration such factors as the age, sex, weight, and condition of the particular subject, and the route of administration.

The anti-resistance agents and/or tyrosine kinase inhibitors can be administered prophylactically or therapeutically. In prophylactic administration, the anti-resistance agents and/or tyrosine kinase inhibitors can be administered in an amount sufficient to induce a response. In therapeutic applications, the anti-resistance agents and/or tyrosine kinase inhibitors are administered to a subject in need thereof in an amount sufficient to elicit a therapeutic effect. The anti-resistance agents and/or tyrosine kinase inhibitors may be administered in a therapeutically effective amount.

For example, a therapeutically effective amount of an anti-resistance agent and/or tyrosine kinase inhibitor or a pharmaceutically acceptable salt thereof, may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The anti-resistance agents and/or tyrosine kinase inhibitors can be administered by methods well known in the art as described in Donnelly et al. (Ann. Rev. Immunol. 1997, 15, 617-648); Feigner et al. (U.S. Pat. No. 5,580,859, issued Dec. 3, 1996); Feigner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The anti-resistance agents and/or tyrosine kinase inhibitors can be complexed to particles or beads that can be administered to an individual, for example, using a vaccine gun. One skilled in the art would know that the choice of a pharmaceutically acceptable carrier, including a physiologically acceptable compound, depends, for example, on the route of administration.

The anti-resistance agents and/or tyrosine kinase inhibitors can be delivered via a variety of routes. Typical delivery routes include parenteral administration, e.g., intradermal, intramuscular or subcutaneous delivery. Other routes include oral administration, intranasal, intravaginal, transdermal, intravenous, intraarterial, intratumoral, intraperitoneal, and epidermal routes. In some embodiments, the anti-resistance agents and/or tyrosine kinase inhibitors are administered intravenously, intraarterially, or intraperitoneally to the subject.

The anti-resistance agents and/or tyrosine kinase inhibitors can be a liquid preparation such as a suspension, syrup, or elixir. The anti-resistance agents and/or tyrosine kinase inhibitors can be incorporated into liposomes, microspheres, or other polymer matrices (such as by a method described in Feigner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The anti-resistance agents and/or tyrosine kinase inhibitors may be used as a vaccine. The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation can be carried out via a minimally invasive device.

In some embodiments, the anti-resistance agents and/or tyrosine kinase inhibitors are administered in a controlled release formulation. The anti-resistance agents and/or tyrosine kinase inhibitors may be released into the circulation, for example. In some embodiments, the anti-resistance agents and/or tyrosine kinase inhibitors may be released over a period of at least about 1 day, at least about 2 days, at least about 3 days, at least about 4 days, at least about 5 days, at least about 6 days, at least about 7 days, at least about 1 week, at least about 1.5 weeks, at least about 2 weeks, at least about 2.5 weeks, at least about 3.5 weeks, at least about 4 weeks, or at least about 1 month.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable dosage forms may also be used for the purposes of formulation.

In some embodiments, the formulation includes agents, such as an excipient, a buffer, an isotonicity agent, a preservative, a surfactant, and preferably zinc. The formulation can also include an excipient or agent for polypeptide stabilization, such as a buffer, a reducing agent, a bulk protein, or a carbohydrate. Bulk proteins useful in formulating at least one polypeptide compositions include albumin, protamine, or the like. Typical carbohydrates useful in formulating at least one polypeptide include sucrose, mannitol, lactose, trehalose, glucose, or the like. The bispecific antibody formulation can also include a surfactant, which can reduce or prevent surface-induced aggregation of the at least one polypeptide caused by atomization of the solution in forming an aerosol. Various conventional surfactants can be employed, such as polyoxyethylene fatty acid esters and alcohols, and polyoxyethylene sorbital fatty acid esters. Amounts will generally range between about 0.001 and 4% by weight of the formulation. Especially preferred surfactants for purposes of this invention are polyoxyethylene sorbitan mono-oleate, polysorbate 80, polysorbate 20, or the like. Additional agents known in the art for formulation of a polypeptide, such as antibody protein, can also be included in the formulation.

### Examples

The Example in this specification is not intended to, and should not be used to, limit the invention; they are provided only to illustrate the invention.

### Example 1. Treatment with EGFR Degrader in Combination with Osimertinib Enhanced Antitumor Activity in Osimertinib-Resistant Mouse Xenograft

The EGFR degrader, bispecific EGFR×NRP1 antibody, has been found to degrade the oncogenic receptor EGFR via lysosomes and reduces in vitro cell viability. It has been also found that the EGFR degrader significantly inhibits tumor growth in Osimertinib-sensitive (H1975), Osimertinib-resistant (H1975-OR), and Osimertinib-refractory (H1975-HGF) xenograft mouse models, respectively, which all mouse xenografts have EGFR double mutations (T970M/L858R) (unpublished data). Osimertinib-resistant xenograft mouse (H1975-OR) used herein did not show anti-tumor activity at Osimertinib dose of 3 mg/kg.

To investigate whether the co-treatment with the EGFR degrader and a therapeutic agent enhances the therapeutic efficacy of the therapeutic agent in a cancer that is resistant to the therapeutic agent, Osimertinib-resistant mouse xenograft model (H1975-OR) was treated with Panitumumab (10 mg/kg, i.p., QW) plus Osimertinib (10 mg/kg, i.p., QD), EGFR degrader (13.74 mg/kg, i.p., QW) plus Osimertinib (10 mg/kg), Osimertinib only, or IgG1 isotype (5 mg/kg, i.p., QW), respectively. Panitumumab is a fully humanized anti-EGFR monoclonal antibody, which binds to EGFR and block the EGFR signaling. Osimertinib is an EGFR tyrosine kinase inhibitor.

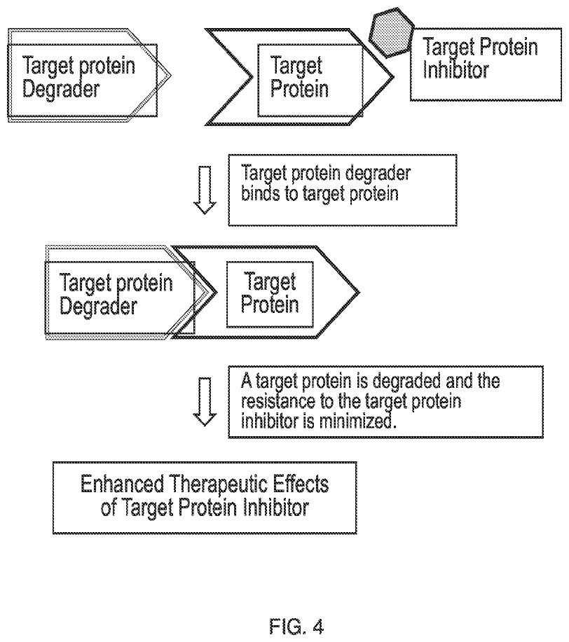

As shown in FIG. 3, the synergistic effect is not found in the Osimertinib-resistant tumor obtained from the mice treated with Panitumumab and Osimertinib. This result suggests that the EGFR signaling by the co-treatment with Osimertinib and Panitumumab is inhibited in the Osimertinib-resistant tumor and the resistance to Osimertinib was sustained. In comparison, the Osimertinib-resistant mice treated with the EGFR degrader and Osimertinib showed the greater tumor regression. The result indicates that EGFR^{T790M/L85SR} degradation by the EGFR degrader via lysosomes and the subsequence removal of EGFR^{T790M/LS58R} produced the high-efficiency synergistic effect on the Osimertinib-resistant tumor. The combination therapy with a therapeutic agent and the target protein degrader enhanced the therapeutic effects of the therapeutic agent in the therapeutic agent-resistant mouse. Taken together, the combination therapy disclosed herein would be a new approach to overcome the drug resistance and to enhance the therapeutic efficacy in the cancer treatment. The schematic illustrating the combination therapy to enhance the therapeutic effect of the therapeutic agent in a patient having a cancer that is resistant to a therapeutic agent is shown in FIG. 4.

### Example 2. Treatment with EGFR Degrader in Combination with Osimertinib Enhanced Anti-Tumor Activity in an NRP1-Overexpressing Mouse Xenograft Model

In this example, a modified version of the H1975 xenograft model was used in which the tumor cells were engineered to overexpress NRP1. In this xenograft model, referred to as H1975-NRP1 OE, the tumor cells are EGFR^{L858R/T790M}, NRP1⁺⁺⁺, EGFR⁺ and Osimertinib refractory. Regarding the latter, treatment of the parental H1975 model with 3 mg/kg Osimertinib showed strong and persistent tumor regression whereas treatment of the H1975-NRP1 OE model with the same dose of Osimertinib led to significant tumor progression, suggesting that NRP1 overexpression induced at least some resistance to Osimertinib treatment.

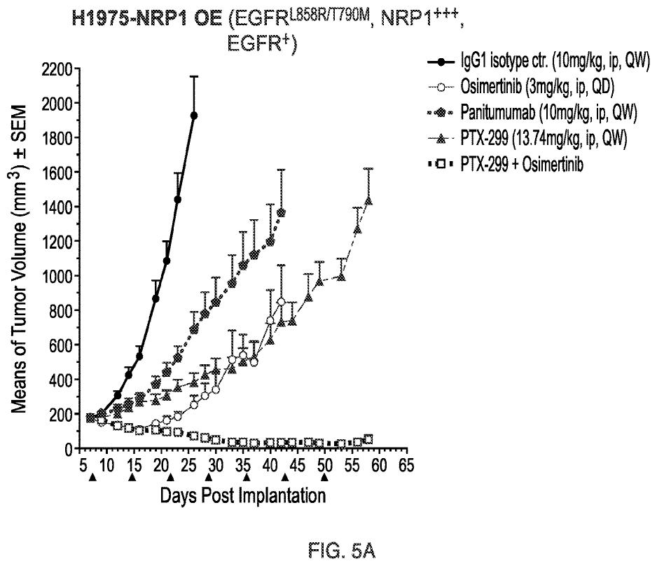

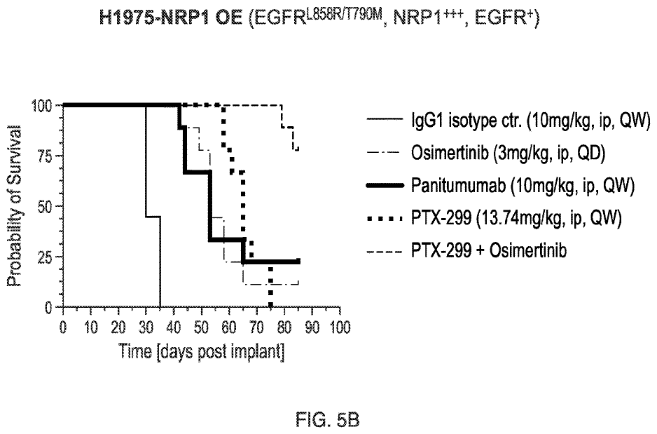

To investigate whether the co-treatment with the EGFR degrader and Osimertinib enhances the therapeutic efficacy of Osimertinib in the H1975-NRP1 OE xenograft model, mice were treated with Panitumumab (10 mg/kg, i.p., QW), EGFR degrader (13.74 mg/kg, i.p., QW), Osimertinib (3 mg/kg, i.p., QD), EGFR degrader plus Osimertinib or IgG1 isotype control (10 mg/kg, i.p., QW), respectively. Results for tumor volume over time are shown in FIG. 5A. Results for probability of survival over time are shown in FIG. 5B.

The results demonstrated that treatment with the EGFR degrader alone resulted in significantly better tumor growth inhibition in this model than treatment with panitumumab alone. Moreover, the combination of the EGFR degrader with Osimertinib led to strong synergism, resulting in tumor regression. While not intending to be limited by mechanism, this suggests that removal of the EGFR mutant via degradation, mediated by the EGFR degrader, is required for the best combination effect for tumor growth inhibition. Treatment with the EGFR degrader alone also led to improved median survival compared to treatment with panitumumab or Osimertinib alone and, again, combination of the EGFR degrader with Osimertinib led to strong synergism, resulting in significantly improved survival of the mice.

Thus, in summary, similar to the results observed in Example 1, the EGFR degrader in combination with Osimertinib was the most effective treatment for inhibiting tumor growth and improving median survival in this NRP1-overexpressing xenograft model.

### Example 3. Treatment with EGFR Degrader in Combination with Therapeutic Agents Enhanced Anti-Tumor Activity in an HGF-Overexpressing Mouse Xenograft Model

In this example, a modified version of the H1975 xenograft model was used in which the tumor cells were engineered to overexpress hepatocyte growth factor (HGF), the ligand for cMET. The HGF/cMET pathway was demonstrated to be active in the H1975 cells by inhibiting the pathway with either Crizotinib, a small molecule inhibitor, which blocked cMET pathway signaling, or Amivantamab, an EGFR×cMET bispecific antibody construct, which blocked cMET pathway signaling and led to cMET degradation (data not shown). The H1975-HGF cell line was prepared by recombinantly engineering the H1975 cell line to express HGF by standard methods. Thus, the H1975-HGF xenograft model represents a dual activation model in which both the EGFR and cMET pathways are constitutively activated.

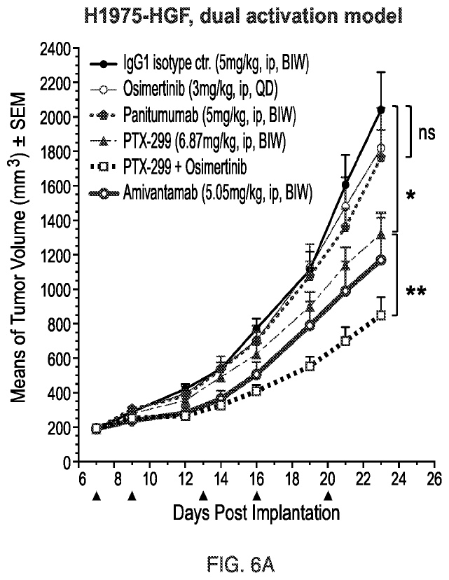

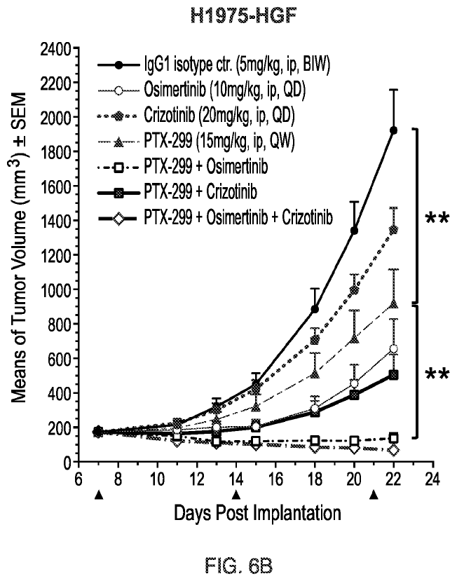

In a first set of experiments, to investigate whether the co-treatment with the EGFR degrader and Osimertinib enhances the therapeutic efficacy of Osimertinib in the H1975-HGF xenograft model, mice were treated with Panitumumab (5 mg/kg, i.p., BIW), EGFR degrader (6.87 mg/kg, i.p., BIW), Amivantamab (5.05 mg/kg, i.p., BIW), Osimertinib (3 mg/kg, i.p., QD), EGFR degrader plus Osimertinib or IgG1 isotype control (5 mg/kg, i.p., BIW), respectively. Results for tumor volume over time are shown in FIG. 6A. The results demonstrated that the H1975-HGF model exhibited resistance to both panitumumab monotherapy and low-dose Osimertinib monotherapy, whereas the EGFR degrader monotherapy and the Amivantamab monotherapy showed similar tumor growth inhibition. The combination of the EGFR degrader plus Osimertinib, however, led to the most tumor growth inhibition over time.

In a second set of experiments, the effect of co-treatment with the EGFR degrader, Osimertinib and the cMET inhibitor Crizotinib was investigated. Mice were treated with Osimertinib (10 mg/kg, i.p., QD), Crizotinib (20 mg/kg, i.p., QD), EGFR degrader (15 mg/kg, i.p., QW), EGFR degrader plus Osimertinib, EGFR degrader plus Crizotinib, EGFR degrader plus Osimertinib and Crizotinib, or IgG1 isotype control (5 mg/kg, i.p., BIW), respectively. Results for tumor volume over time are shown in FIG. 6B. The results demonstrated that Crizotinib monotherapy in the H1975-HGF model had limited efficacy, High-dose Osimertinib monotherapy also did not prevent tumor growth over time. EGFR degrader monotherapy did exhibit tumor growth inhibition, while combination of the EGFR degrader with either Crizotinib or Osimertinib exhibited enhanced tumor growth inhibition as compared to monotherapy with any of the agents. The triple combination of the EGFR degrader, Osimertinib and Crizotinib demonstrated the best effect over time.

Thus, in summary, similar to the results observed in Examples 1 and 2, the EGFR degrader in combination with either Osimertinib or Crizotinib, or with both agents together, significantly improved tumor growth inhibition in this HGF-overexpressing xenograft model of dual EGFR/cMET pathway activation.

### Example 4. Treatment with EGFR Degrader in Combination with KRAS Inhibitor Enhanced Anti-Tumor Activity in a Mutant KRAS Mouse Xenograft Model

In this example, a mutant KRAS mouse xenograft model was used to examine the effect of the EGFR degrader, alone or in combination with a KRAS inhibitor, on tumor growth in the mutant KRAS model H358. In the H358 model, tumor cells are KRAS^{G12C}, NRP1⁺, EGFR⁺ and responsive to Sotorasib, an irreversible KRAS G12C selective inhibitor.

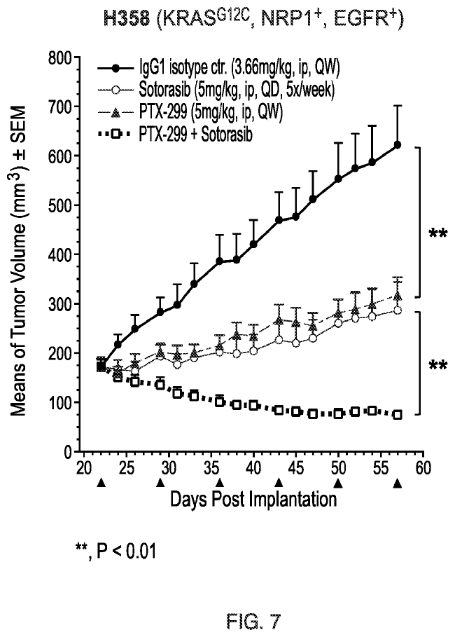

Mice were treated with Sotorasib (5 mg/kg, i.p., QD, 5×/wk), EGFR degrader (5 mg/kg, i.p., QW), EGFR degrader plus Sotorasib or IgG1 isotype control (3.66 mg/kg, i.p., QW), respectively. Results for tumor volume over time are shown in FIG. 7. The results demonstrated that both Sotorasib monotherapy and EGFR degrader monotherapy exhibited strong anti-tumor activity of approximately equivalent strength, whereas the combination of the EGFR degrader and Sotorasib demonstrated synergistic effects, leading to the most tumor regression. These results further confirmed the effects observed and reported in Examples 1-3 showing the effectiveness of combining the EGFR degrader with a therapeutic agent targeting a pathway of the tumor cells to thereby enhance anti-tumor activity of both agents.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

TABLE 3
SEQUENCE LISTING SUMMARY

| SEQ ID | |
|---|---|
| NO: | Sequence |
| 1 | QVQLQESGPGLVKPSETLSLTCTVSGGSVSSGDYYWTWIRQSPGKGLEWIGHIYYSGNTNYNPSLK |
| | SRLTISIDTSKTQFSLKLSSVTAADTAIYYCVRDRVTGAFDIWGQGTMVTVSSASTKGPSVFPLAPCS |
| | RSTSESTAALGCLVKDYFPEPVTVSWNSGALTSGVHTFPAVLQSSGLYSLSSVVTVPSSNFGTQTYT |
| | CNVDHKPSNTKVDKTVERKCCVECPPCPAPPVAGPSVFLFPPKPKDTLMISRTPEVTCVVVDVSHE |
| | DPEVQFNWYVDGVEVHNAKTKPREEQFNSTFRVVSVLTVVHQDWLNGKEYKCKVSNKGLPAPIE |
| | KTISKTKGQPREPQVYTLPPSREEMTKNQVSLTCLVKGFYPSDIAVEWESNGQPENNYKTTPPMLD |
| | SDGSFFLYSKLTVDKSRWQQGNVFSCSVMHEALHNHYTQKSLSLSPGKGGGGSGGGGSGGGGS |
| | HTPGNSKPTRTPRR |
| 2 | QVQLQESGPGLVKPSETLSLTCTVSGGSVSSGDYYWTWIRQSPGKGLEWIGHIYYSGNTNYNPSLK |
| | SRLTISIDTSKTQFSLKLSSVTAADTAIYYCVRDRVTGAFDIWGQGTMVTVSSASTKGPSVFPLAPCS |
| | RSTSESTAALGCLVKDYFPEPVTVSWNSGALTSGVHTFPAVLQSSGLYSLSSVVTVPSSNFGTQTYT |
| | CNVDHKPSNTKVDKTVERKCCVECPPCPAPPVAGPSVFLFPPKPKDTLMISRTPEVTCVVVDVSHE |
| | DPEVQFNWYVDGVEVHNAKTKPREEQFNSTFRVVSVLTVVHQDWLNGKEYKCKVSNKGLPAPIE |
| | KTISKTKGQPREPQVYTLPPSREEMTKNQVSLTCLVKGFYPSDIAVEWESNGQPENNYKTTPPMLD |
| | SDGSFFLYSKLTVDKSRWQQGNVFSCSVMHEALHNHYTQKSLSLSPGKGGGGSGGGGSGGGGS |
| | EVQLVESGGGLVQPGGSLRLSCAASGFTFSSYAMSWVRQAPGKGLEWVSQISPAGGYTNYADSV |
| | KGRFTISADTSKNTAYLQMNSLRAEDTAVYYCARGELPYYRMSKVMDVWGQGTLVTVSSGGGGS |
| | GGGGSGGGGSDIQMTQSPSSLSASVGDRVTITCRASQYFSSYLAWYQQKPGKAPKLLIYGASSRAS |
| | GVPSRFSGSGSGTDFTLTISSLQPEDFATYYCQQYLGSPPTFGQGTKVEIK |
| 3 | QVQLQESGPGLVKPSETLSLTCTVSGGSVSSGDYYWTWIRQSPGKGLEWIGHIYYSGNTNYNPSLK |
| | SRLTISIDTSKTQFSLKLSSVTAADTAIYYCVRDRVTGAFDIWGQGTMVTVSSASTKGPSVFPLAPCS |
| | RSTSESTAALGCLVKDYFPEPVTVSWNSGALTSGVHTFPAVLQSSGLYSLSSVVTVPSSNFGTQTYT |
| | CNVDHKPSNTKVDKTVERKCCVECPPCPAPPVAGPSVFLFPPKPKDTLMISRTPEVTCVVVDVSHE |
| | DPEVQFNWYVDGVEVHNAKTKPREEQFNSTFRVVSVLTVVHQDWLNGKEYKCKVSNKGLPAPIE |
| | KTISKTKGQPREPQVYTLPPSREEMTKNQVSLTCLVKGFYPSDIAVEWESNGQPENNYKTTPPMLD |
| | SDGSFFLYSKLTVDKSRWQQGNVFSCSVMHEALHNHYTQKSLSLSPGKGGGGSGGGGSGGGGS |
| | EVQLVESGGGLVQPGGSLRLSCAASGFTFSSYAMSWVRQAPGKGLEWVSQISPAGGYTNYADSV |
| | KGRFTISADTSKNTAYLQMNSLRAEDTAVYYCARGELPYFRMSKVMDVWGQGTLVTVSSGGGGS |
| | GGGGSGGGGSDIQMTQSPSSLSASVGDRVTITCRASQFLSSYLAWYQQKPGKAPKLLIYGASARA |
| | SGVPSRFSGSGSGTDFTLTISSLQPEDFATYYCQQYLASPATFGQGTKVEIK |
| 4 | QVQLQESGPGLVKPSETLSLTCTVSGGSVSSGDYYWTWIRQSPGKGLEWIGHIYYSGNTNYNPSLK |
| | SRLTISIDTSKTQFSLKLSSVTAADTAIYYCVRDRVTGAFDIWGQGTMVTVSSASTKGPSVFPLAPCS |
| | RSTSESTAALGCLVKDYFPEPVTVSWNSGALTSGVHTFPAVLQSSGLYSLSSVVTVPSSNFGTQTYT |
| | CNVDHKPSNTKVDKTVERKCCVECPPCPAPPVAGPSVFLFPPKPKDTLMISRTPEVTCVVVDVSHE |
| | DPEVQFNWYVDGVEVHNAKTKPREEQFNSTFRVVSVLTVVHQDWLNGKEYKCKVSNKGLPAPIE |
| | KTISKTKGQPREPQVYTLPPSREEMTKNQVSLTCLVKGFYPSDIAVEWESNGQPENNYKTTPPMLD |
| | SDGSFFLYSKLTVDKSRWQQGNVFSCSVMHEALHNHYTQKSLSLSPGKGGGGSGGGGSGGGGS |
| | EVQLVESGGGLVQPGGSLRLSCAASGFTFSSYAMSWVRQAPGKGLEWVSQISPAGGYTNYADSV |
| | KGRFTISADTSKNTAYLQMNSLRAEDTAVYYCARGELPYYQMSKVMDVWGQGTLVTVSSGGGG |
| | SGGGGSGGGGSDIQMTQSPSSLSASVGDRVTITCRASQYFSSYLAWYQQKPGKAPKLLIYGASAR |
| | ASGVPSRFSGSGSGTDFTLTISSLQPEDFATYYCQQYLASPATFGQGTKVEIK |
| 5 | QVQLQESGPGLVKPSETLSLTCTVSGGSVSSGDYYWTWIRQSPGKGLEWIGHIYYSGNTNYNPSLK |
| | SRLTISIDTSKTQFSLKLSSVTAADTAIYYCVRDRVTGAFDIWGQGTMVTVSSASTKGPSVFPLAPCS |
| | RSTSESTAALGCLVKDYFPEPVTVSWNSGALTSGVHTFPAVLQSSGLYSLSSVVTVPSSNFGTQTYT |
| | CNVDHKPSNTKVDKTVERKCCVECPPCPAPPVAGPSVFLFPPKPKDTLMISRTPEVTCVVVDVSHE |
| | DPEVQFNWYVDGVEVHNAKTKPREEQFNSTFRVVSVLTVVHQDWLNGKEYKCKVSNKGLPAPIE |
| | KTISKTKGQPREPQVYTLPPSREEMTKNQVSLTCLVKGFYPSDIAVEWESNGQPENNYKTTPPMLD |
| | SDGSFFLYSKLTVDKSRWQQGNVFSCSVMHEALHNHYTQKSLSLSPGKGGGGSGGGGSGGGGS |
| | EVQLVESGGGLVQPGGSLRLSCAASGFTFSSYAMSWVRQAPGKGLEWVSQISPAGGYTNYADSV |
| | KGRFTISADTSKNTAYLQMNSLRAEDTAVYYCARGELPFFRMSQVMDVWGQGTLVTVSSGGGG |
| | SGGGGSGGGGSDIQMTQSPSSLSASVGDRVTITCRASQFLSSYLAWYQQKPGKAPKLLIYGASAR |
| | ASGVPSRFSGSGSGTDFTLTISSLQPEDFATYYCQQYLASPATFGQGTKVEIK |
| 6 | QVQLQESGPGLVKPSETLSLTCTVSGGSVSSGDYYWTWIRQSPGKGLEWIGHIYYSGNTNYNPSLK |
| | SRLTISIDTSKTQFSLKLSSVTAADTAIYYCVRDRVTGAFDIWGQGTMVTVSSASTKGPSVFPLAPCS |
| | RSTSESTAALGCLVKDYFPEPVTVSWNSGALTSGVHTFPAVLQSSGLYSLSSVVTVPSSNFGTQTYT |
| | CNVDHKPSNTKVDKTVERKCCVECPPCPAPPVAGPSVFLFPPKPKDTLMISRTPEVTCVVVDVSHE |
| | DPEVQFNWYVDGVEVHNAKTKPREEQFNSTFRVVSVLTVVHQDWLNGKEYKCKVSNKGLPAPIE |
| | KTISKTKGQPREPQVYTLPPSREEMTKNQVSLTCLVKGFYPSDIAVEWESNGQPENNYKTTPPMLD |
| | SDGSFFLYSKLTVDKSRWQQGNVFSCSVMHEALHNHYTQKSLSLSPGKGGGGSGGGGSGGGGS |
| | EVQLVESGGGLVQPGGSLRLSCAASGFTFSSYAMSWVRQAPGKGLEWVSQISPAGGYTNYADSV |
| | KGRFTISADTSKNTAYLQMNSLRAEDTAVYYCARGELPYFRMSKVMDVWGQGTLVTVSSGGGGS |
| | GGGGSGGGGSDIQMTQSPSSLSASVGDRVTITCRASQFLSSFLAWYQQKPGKAPKLLIYGASARAS |
| | GVPSRFSGSGSGTDFTLTISSLQPEDFATYYCQQYLASPATFGQGTKVEIK |
| 7 | QVQLQESGPGLVKPSETLSLTCTVSGGSVSSGDYYWTWIRQSPGKGLEWIGHIYYSGNTNYNPSLK |
| | SRLTISIDTSKTQFSLKLSSVTAADTAIYYCVRDRVTGAFDIWGQGTMVTVSSASTKGPSVFPLAPSS |
| | KSTSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGKGGGGSGGGGSGG |
| | GGSEVQLVESGGGLVQPGGSLRLSCAASGFTFSSYAMSWVRQAPGKGLEWVSQISPAGGYTNYA |
| | DSVKGRFTISADTSKNTAYLQMNSLRAEDTAVYYCARGELPYFRMSKVMDVWGQGTLVTVSSGG |
| | GGSGGGGSGGGGSDIQMTQSPSSLSASVGDRVTITCRASQFLSSYLAWYQQKPGKAPKLLIYGAS |
| | ARASGVPSRFSGSGSGTDFTLTISSLQPEDFATYYCQQYLASPATFGQGTKVEIK |
| 8 | QVQLQESGPGLVKPSETLSLTCTVSGGSVSSGDYYWTWIRQSPGKGLEWIGHIYYSGNTNYNPSLK |
| | SRLTISIDTSKTQFSLKLSSVTAADTAIYYCVRDRVTGAFDIWGQGTMVTVSSASTKGPSVFPLAPSS |
| | KSTSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGKGGGGSGGGGSGG |
| | GGSEVQLVESGGGLVQPGGSLRLSCAASGFTFSSYAMSWVRQAPGKGLEWVSQISPAGGYTNYA |
| | DSVKGRFTISADTSKNTAYLQMNSLRAEDTAVYYCARGELPYFRMSKVMDVWGQGTLVTVSSGD |
| | GSSGGGGASDIQMTQSPSSLSASVGDRVTITCRASQFLSSYLAWYQQKPGKAPKLLIYGASARASG |
| | VPSRFSGSGSGTDFTLTISSLQPEDFATYYCQQYLASPATFGQGTKVEIK |
| 9 | QVQLQESGPGLVKPSETLSLTCTVSGGSVSSGDYYWTWIRQSPGKGLEWIGHIYYSGNTNYNPSLK |
| | SRLTISIDTSKTQFSLKLSSVTAADTAIYYCVRDRVTGAFDIWGQGTMVTVSSASTKGPSVFPLAPSS |
| | KSTSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGKGGGGSGGGGSGG |
| | GGSEVQLVESGGGLVQPGGSLRLSCAASGFTFSSYAMSWVRQAPGKGLEWVSQISPAGGYTNYA |
| | DSVKGRFTISADTSKNTAYLQMNSLRAEDTAVYYCARGELPYFRMSKVMDVWGQGTLVTVSSGG |
| | GGSGGGGSGGGGSGGGGSDIQMTQSPSSLSASVGDRVTITCRASQFLSSYLAWYQQKPGKAPKL |
| | LIYGASARASGVPSRFSGSGSGTDFTLTISSLQPEDFATYYCQQYLASPATFGQGTKVEIK |
| 10 | QVQLQESGPGLVKPSETLSLTCTVSGGSVSSGDYYWTWIRQSPGKGLEWIGHIYYSGNTNYNPSLK |
| | SRLTISIDTSKTQFSLKLSSVTAADTAIYYCVRDRVTGAFDIWGQGTMVTVSSASTKGPSVFPLAPSS |
| | KSTSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGKGGGGSGGGGSGG |
| | GGSDIQMTQSPSSLSASVGDRVTITCRASQFLSSYLAWYQQKPGKAPKLLIYGASARASGVPSRFS |
| | GSGSGTDFTLTISSLQPEDFATYYCQQYLASPATFGQGTKVEIKGGGGSGGGGSGGGGSEVQLVES |
| | GGGLVQPGGSLRLSCAASGFTFSSYAMSWVRQAPGKGLEWVSQISPAGGYTNYADSVKGRFTIS |
| | ADTSKNTAYLQMNSLRAEDTAVYYCARGELPYFRMSKVMDVWGQGTLVTVSS |
| 11 | QVQLQESGPGLVKPSETLSLTCTVSGGSVSSGDYYWTWIRQSPGKGLEWIGHIYYSGNTNYNPSLK |
| | SRLTISIDTSKTQFSLKLSSVTAADTAIYYCVRDRVTGAFDIWGQGTMVTVSSASTKGPSVFPLAPSS |
| | KSTSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGKGGGGSGGGGSGG |
| | GGSEVQLLESGGGLVQPGGSLRLSCAASGFTFSSYAMSWVRQAPGKGLEWVSQISPAGGYTNYA |
| | DSVKGRFTISRDNSKNTLYLQMNSLRAEDTAVYYCARGELPYFRMSKVMDVWGQGTLVTVSSGG |
| | GGSGGGGSGGGGSEIVLTQSPGTLSLSPGERATLSCRASQFLSSYLAWYQQKPGQAPRLLIYGASA |
| | RASGIPDRFSGSGSGTDFTLTISRLEPEDFAVYYCQQYLASPATFGQGTKVEIK |
| 12 | DIQMTQSPSSLSASVGDRVTITCQASQDISNYLNWYQQKPGKAPKLLIYDASNLETGVPSRFSGSG |
| | SGTDFTFTISSLQPEDIATYFCQHFDHLPLAFGGGTKVEIKRTVAAPSVFIFPPSDEQLKSGTASVVCL |
| | LNNFYPREAKVQWKVDNALQSGNSQESVTEQDSKDSTYSLSSTLTLSKADYEKHKVYACEVTHQG |
| | LSSPVTKSFNRGEC |
| 13 | GGGGS |
| 14 | CAAGTGCAGCTGCAGGAATCCGGCCCCGGGCTTGTGAAGCCTAGCGAAACACTCTCGCTCACC |
| | TGTACTGTCAGCGGTGGATCAGTGTCCTCCGGCGATTACTACTGGACCTGGATTCGGCAGAGC |
| | CCTGGAAAGGGACTGGAGTGGATCGGACACATCTACTACTCCGGGAACACTAACTACAACCC |
| | GTCGTTGAAGTCCAGACTGACGATCAGTATCGATACCTCCAAGACCCAGTTCAGCCTGAAGCT |
| | GAGCTCAGTGACAGCCGCCGACACTGCAATCTACTACTGCGTGCGGGACAGAGTGACCGGAG |
| | CCTTCGACATCTGGGGCCAGGGAACCATGGTCACTGTGTCGTCTGCGTCAACCAAGGGTCCGT |
| | CCGTGTTTCCCCTGGCCCCGTGCTCGCGGAGCACCTCCGAGTCCACTGCCGCCTTGGGCTGCCT |
| | GGTCAAAGACTACTTCCCTGAACCCGTGACTGTCAGCTGGAACTCCGGAGCTCTGACCTCGGG |
| | AGTGCACACCTTCCCGGCCGTGCTGCAATCGAGCGGCCTCTACTCCCTGTCCTCCGTCGTGACC |
| | GTGCCATCATCAAACTTCGGAACCCAAACTTATACGTGCAACGTCGACCACAAGCCCTCCAATA |
| | CCAAAGTCGACAAGACCGTGGAGAGGAAATGCTGCGTGGAGTGTCCGCCTTGCCCCGCGCCG |
| | CCGGTGGCCGGACCTAGCGTGTTCCTGTTCCCGCCGAAGCCAAAGGACACTCTCATGATCTCC |
| | CGCACCCCTGAAGTCACTTGCGTCGTGGTGGACGTTTCCCACGAGGATCCCGAAGTGCAGTTC |
| | AATTGGTACGTGGACGGGGTGGAAGTACATAACGCCAAGACCAAGCCCAGGGAAGAACAGT |
| | TTAACTCCACCTTCCGGGTGGTGTCGGTGCTCACTGTGGTGCATCAGGATTGGCTCAATGGAA |
| | AGGAGTACAAGTGCAAAGTGTCGAACAAGGGTCTGCCCGCTCCTATTGAAAAGACCATTTCCA |
| | AAACCAAGGGACAGCCCAGAGAGCCTCAGGTCTACACCCTGCCTCCGAGCCGCGAGGAAATG |
| | ACCAAGAACCAAGTGTCTCTGACTTGCCTCGTGAAGGGATTCTACCCCTCCGATATCGCGGTG |
| | GAGTGGGAGAGCAACGGGCAGCCAGAGAACAACTATAAGACCACCCCGCCTATGCTGGACTC |
| | CGATGGCTCCTTCTTCTTGTACTCGAAGCTGACCGTGGACAAGTCCCGCTGGCAACAGGGAAA |
| | CGTGTTCAGCTGTAGCGTGATGCACGAAGCCCTGCACAACCACTACACCCAGAAGTCCCTGTC |
| | GCTTTCCCCCGGGAAGGGCGGTGGCGGATCCGGCGGCGGGGGCAGCGGGGGCGGCGGTTC |
| | CCATACCCCGGGGAACTCAAAGCCCACCCGGACTCCACGGCGC |
| 15 | CAGGTTCAGCTGCAAGAGTCTGGCCCTGGCCTGGTCAAGCCTTCCGAAACACTGTCTCTGACC |
| | TGCACCGTGTCTGGCGGCTCTGTGTCCTCTGGCGATTACTACTGGACCTGGATCCGGCAGTCTC |
| | CTGGCAAAGGCCTGGAATGGATCGGCCACATCTACTACTCCGGCAACACCAACTACAACCCCA |
| | GCCTGAAGTCCCGGCTGACCATCTCCATCGACACCAGCAAGACCCAGTTCTCCCTGAAGCTGT |
| | CCTCTGTGACCGCCGCTGATACCGCCATCTACTATTGCGTGCGGGACAGAGTGACCGGCGCCT |
| | TTGATATTTGGGGCCAGGGCACCATGGTCACCGTGTCCAGTGCTTCTACCAAGGGACCCAGCG |
| | TGTTCCCTCTGGCTCCTTGCTCCAGATCCACCTCCGAGTCTACAGCTGCTCTGGGCTGTCTGGT |
| | CAAGGACTACTTCCCTGAGCCTGTGACCGTGTCCTGGAATTCTGGCGCTCTGACATCTGGCGT |
| | GCACACCTTTCCAGCTGTGCTGCAGTCTAGCGGCCTGTACTCTCTGTCCTCCGTCGTGACCGTG |
| | CCTTCCTCTAACTTTGGCACCCAGACCTACACCTGTAATGTGGACCACAAGCCATCCAACACCA |
| | AGGTGGACAAGACCGTGGAACGGAAGTGCTGCGTGGAATGCCCTCCTTGTCCTGCTCCTCCTG |
| | TGGCTGGCCCTTCCGTGTTTCTGTTCCCTCCAAAGCCTAAGGACACCCTGATGATCTCTCGGAC |
| | CCCTGAAGTGACCTGCGTGGTGGTGGATGTGTCTCACGAGGATCCCGAGGTGCAGTTCAATT |
| | GGTACGTGGACGGCGTGGAAGTGCACAACGCCAAGACCAAGCCTAGAGAGGAACAGTTCAA |
| | CTCCACCTTCAGAGTGGTGTCCGTGCTGACCGTGGTGCATCAGGATTGGCTGAACGGCAAAG |
| | AGTACAAGTGCAAGGTGTCCAACAAGGGCCTGCCTGCTCCTATCGAAAAGACCATCTCTAAGA |
| | CCAAGGGGCAGCCCCGGGAACCTCAGGTTTACACACTGCCTCCAAGCCGGGAAGAGATGACC |
| | AAGAACCAGGTGTCCCTGACCTGTCTCGTGAAGGGCTTCTACCCCTCCGATATCGCCGTGGAA |
| | TGGGAGTCTAATGGCCAGCCTGAGAACAACTACAAGACCACACCTCCTATGCTGGACTCCGAC |
| | GGCTCATTCTTCCTGTACTCCAAGCTGACAGTGGACAAGTCCAGATGGCAGCAGGGCAACGTG |
| | TTCTCCTGCTCCGTGATGCACGAGGCCCTGCACAATCACTACACCCAGAAGTCCCTGTCTCTGT |
| | CCCCTGGAAAAGGCGGCGGAGGATCTGGCGGAGGCGGTAGCGGTGGTGGCGGATCTGAAGT |
| | TCAGCTGGTTGAATCTGGCGGCGGACTGGTTCAACCAGGCGGATCTCTGAGACTGTCTTGTGC |
| | CGCCTCCGGCTTCACCTTCTCCTCTTACGCTATGTCCTGGGTCCGACAGGCCCCAGGCAAAGGA |
| | TTGGAGTGGGTGTCCCAGATCTCTCCCGCTGGCGGCTACACCAATTACGCCGACTCTGTGAAG |
| | GGCAGATTCACCATCTCTGCCGACACCTCCAAGAACACCGCCTACCTGCAGATGAACTCCCTGA |
| | GAGCCGAGGACACCGCCGTGTACTATTGTGCTAGAGGCGAGCTGCCCTACTACCGGATGTCC |
| | AAAGTGATGGACGTGTGGGGACAGGGAACCCTCGTGACAGTTTCTAGCGGTGGCGGAGGTA |
| | GCGGAGGCGGTGGAAGCGGCGGAGGCGGAAGTGATATTCAGATGACCCAGTCTCCTTCCAG |
| | CCTGTCCGCTTCTGTGGGCGATAGAGTGACCATCACCTGTCGGGCCTCTCAGTACTTCTCCTCC |
| | TACCTGGCTTGGTATCAGCAGAAGCCTGGCAAGGCCCCTAAGCTGCTGATCTACGGCGCCTCC |
| | TCTAGAGCTAGCGGCGTGCCCTCTAGATTCTCCGGATCTGGCTCTGGCACCGACTTTACCCTGA |
| | CAATCTCCAGCCTGCAGCCTGAGGACTTCGCCACCTACTACTGCCAGCAGTACCTGGGCTCTCC |
| | TCCAACCTTTGGCCAGGGAACAAAGGTCGAGATCAAGCGCTGA |
| 16 | CAGGTTCAGCTGCAAGAGTCTGGCCCTGGCCTGGTCAAGCCTTCCGAAACACTGTCTCTGACC |
| | TGCACCGTGTCTGGCGGCTCTGTGTCCTCTGGCGATTACTACTGGACCTGGATCCGGCAGTCTC |
| | CTGGCAAAGGCCTGGAATGGATCGGCCACATCTACTACTCCGGCAACACCAACTACAACCCCA |
| | GCCTGAAGTCCCGGCTGACCATCTCCATCGACACCAGCAAGACCCAGTTCTCCCTGAAGCTGT |
| | CCTCTGTGACCGCCGCTGATACCGCCATCTACTATTGCGTGCGGGACAGAGTGACCGGCGCCT |
| | TTGATATTTGGGGCCAGGGCACCATGGTCACCGTGTCCAGTGCTTCTACCAAGGGACCCAGCG |
| | TGTTCCCTCTGGCTCCTTGCTCCAGATCCACCTCCGAGTCTACAGCTGCTCTGGGCTGTCTGGT |
| | CAAGGACTACTTCCCTGAGCCTGTGACCGTGTCCTGGAATTCTGGCGCTCTGACATCTGGCGT |
| | GCACACCTTTCCAGCTGTGCTGCAGTCTAGCGGCCTGTACTCTCTGTCCTCCGTCGTGACCGTG |
| | CCTTCCTCTAACTTTGGCACCCAGACCTACACCTGTAATGTGGACCACAAGCCATCCAACACCA |
| | AGGTGGACAAGACCGTGGAACGGAAGTGCTGCGTGGAATGCCCTCCTTGTCCTGCTCCTCCTG |
| | TGGCTGGCCCTTCCGTGTTTCTGTTCCCTCCAAAGCCTAAGGACACCCTGATGATCTCTCGGAC |
| | CCCTGAAGTGACCTGCGTGGTGGTGGATGTGTCTCACGAGGATCCCGAGGTGCAGTTCAATT |
| | GGTACGTGGACGGCGTGGAAGTGCACAACGCCAAGACCAAGCCTAGAGAGGAACAGTTCAA |
| | CTCCACCTTCAGAGTGGTGTCCGTGCTGACCGTGGTGCATCAGGATTGGCTGAACGGCAAAG |
| | AGTACAAGTGCAAGGTGTCCAACAAGGGCCTGCCTGCTCCTATCGAAAAGACCATCTCTAAGA |
| | CCAAGGGGCAGCCCCGGGAACCTCAGGTTTACACACTGCCTCCAAGCCGGGAAGAGATGACC |
| | AAGAACCAGGTGTCCCTGACCTGTCTCGTGAAGGGCTTCTACCCCTCCGATATCGCCGTGGAA |
| | TGGGAGTCTAATGGCCAGCCTGAGAACAACTACAAGACCACACCTCCTATGCTGGACTCCGAC |
| | GGCTCATTCTTCCTGTACTCCAAGCTGACAGTGGACAAGTCCAGATGGCAGCAGGGCAACGTG |
| | TTCTCCTGCTCCGTGATGCACGAGGCCCTGCACAATCACTACACCCAGAAGTCCCTGTCTCTGT |
| | CCCCTGGAAAAGGCGGCGGAGGATCTGGCGGAGGCGGTAGCGGTGGTGGCGGATCTGAAGT |
| | TCAGCTGGTTGAATCTGGCGGCGGACTGGTTCAACCAGGCGGATCTCTGAGACTGTCTTGTGC |
| | CGCCTCCGGCTTCACCTTCTCCTCTTACGCTATGTCCTGGGTCCGACAGGCCCCAGGCAAAGGA |
| | TTGGAGTGGGTGTCCCAGATCTCTCCCGCTGGCGGCTACACCAATTACGCCGACTCTGTGAAG |
| | GGCAGATTCACCATCTCTGCCGACACCTCCAAGAACACCGCCTACCTGCAGATGAACTCCCTGA |
| | GAGCCGAGGACACCGCCGTGTACTATTGTGCTAGAGGCGAGCTGCCCTACTTCCGGATGTCCA |
| | AAGTGATGGACGTGTGGGGACAGGGAACCCTCGTGACAGTTTCTAGCGGTGGCGGAGGTAG |
| | CGGAGGCGGTGGAAGCGGCGGAGGCGGAAGTGATATTCAGATGACCCAGTCTCCTTCCAGCC |
| | TGTCCGCTTCTGTGGGCGATAGAGTGACCATCACCTGTCGGGCCTCTCAGTTCCTGTCCTCCTA |
| | CCTGGCTTGGTATCAGCAGAAGCCTGGCAAGGCCCCTAAGCTGCTGATCTACGGCGCTTCTGC |
| | TAGAGCTTCCGGCGTGCCCTCCAGATTTTCTGGCTCTGGATCTGGCACCGACTTTACCCTGACA |
| | ATCTCCAGCCTGCAGCCTGAGGACTTCGCCACCTACTACTGCCAGCAGTACCTGGCCTCTCCTG |
| | CCACATTTGGCCAGGGAACAAAGGTCGAGATCAAGCGCTGA |
| 17 | CAGGTTCAGCTGCAAGAGTCTGGCCCTGGCCTGGTCAAGCCTTCCGAAACACTGTCTCTGACC |
| | TGCACCGTGTCTGGCGGCTCTGTGTCCTCTGGCGATTACTACTGGACCTGGATCCGGCAGTCTC |
| | CTGGCAAAGGCCTGGAATGGATCGGCCACATCTACTACTCCGGCAACACCAACTACAACCCCA |
| | GCCTGAAGTCCCGGCTGACCATCTCCATCGACACCAGCAAGACCCAGTTCTCCCTGAAGCTGT |
| | CCTCTGTGACCGCCGCTGATACCGCCATCTACTATTGCGTGCGGGACAGAGTGACCGGCGCCT |
| | TTGATATTTGGGGCCAGGGCACCATGGTCACCGTGTCCAGTGCTTCTACCAAGGGACCCAGCG |
| | TGTTCCCTCTGGCTCCTTGCTCCAGATCCACCTCCGAGTCTACAGCTGCTCTGGGCTGTCTGGT |
| | CAAGGACTACTTCCCTGAGCCTGTGACCGTGTCCTGGAATTCTGGCGCTCTGACATCTGGCGT |
| | GCACACCTTTCCAGCTGTGCTGCAGTCTAGCGGCCTGTACTCTCTGTCCTCCGTCGTGACCGTG |
| | CCTTCCTCTAACTTTGGCACCCAGACCTACACCTGTAATGTGGACCACAAGCCATCCAACACCA |
| | AGGTGGACAAGACCGTGGAACGGAAGTGCTGCGTGGAATGCCCTCCTTGTCCTGCTCCTCCTG |
| | TGGCTGGCCCTTCCGTGTTTCTGTTCCCTCCAAAGCCTAAGGACACCCTGATGATCTCTCGGAC |
| | CCCTGAAGTGACCTGCGTGGTGGTGGATGTGTCTCACGAGGATCCCGAGGTGCAGTTCAATT |
| | GGTACGTGGACGGCGTGGAAGTGCACAACGCCAAGACCAAGCCTAGAGAGGAACAGTTCAA |
| | CTCCACCTTCAGAGTGGTGTCCGTGCTGACCGTGGTGCATCAGGATTGGCTGAACGGCAAAG |
| | AGTACAAGTGCAAGGTGTCCAACAAGGGCCTGCCTGCTCCTATCGAAAAGACCATCTCTAAGA |
| | CCAAGGGGCAGCCCCGGGAACCTCAGGTTTACACACTGCCTCCAAGCCGGGAAGAGATGACC |
| | AAGAACCAGGTGTCCCTGACCTGTCTCGTGAAGGGCTTCTACCCCTCCGATATCGCCGTGGAA |
| | TGGGAGTCTAATGGCCAGCCTGAGAACAACTACAAGACCACACCTCCTATGCTGGACTCCGAC |
| | GGCTCATTCTTCCTGTACTCCAAGCTGACAGTGGACAAGTCCAGATGGCAGCAGGGCAACGTG |
| | TTCTCCTGCTCCGTGATGCACGAGGCCCTGCACAATCACTACACCCAGAAGTCCCTGTCTCTGT |
| | CCCCTGGAAAAGGCGGCGGAGGATCTGGCGGAGGCGGTAGCGGTGGTGGCGGATCTGAAGT |
| | TCAGCTGGTTGAATCTGGCGGCGGACTGGTTCAACCAGGCGGATCTCTGAGACTGTCTTGTGC |
| | CGCCTCCGGCTTCACCTTCTCCTCTTACGCTATGTCCTGGGTCCGACAGGCCCCAGGCAAAGGA |
| | TTGGAGTGGGTGTCCCAGATCTCTCCCGCTGGCGGCTACACCAATTACGCCGACTCTGTGAAG |
| | GGCAGATTCACCATCTCTGCCGACACCTCCAAGAACACCGCCTACCTGCAGATGAACTCCCTGA |
| | GAGCCGAGGACACCGCCGTGTACTATTGTGCTAGAGGCGAGCTGCCCTACTACCAGATGTCCA |
| | AAGTGATGGACGTGTGGGGACAGGGAACCCTCGTGACAGTTTCTAGCGGTGGCGGAGGTAG |
| | CGGAGGCGGTGGAAGCGGCGGAGGCGGAAGTGATATTCAGATGACCCAGTCTCCTTCCAGCC |
| | TGTCCGCTTCTGTGGGCGATAGAGTGACCATCACCTGTCGGGCCTCTCAGTACTTCTCTTCCTA |
| | TCTGGCATGGTATCAGCAGAAGCCTGGCAAGGCCCCTAAGCTGCTGATCTACGGCGCTTCTGC |
| | TAGAGCTTCCGGCGTGCCCTCCAGATTTTCTGGCTCTGGATCTGGCACCGACTTTACCCTGACA |
| | ATCTCCAGCCTGCAGCCTGAGGACTTCGCCACCTACTACTGCCAGCAGTACCTGGCCTCTCCTG |
| | CCACATTTGGCCAGGGAACAAAGGTCGAGATCAAGCGCTGA |
| 18 | CAGGTTCAGCTGCAAGAGTCTGGCCCTGGCCTGGTCAAGCCTTCCGAAACACTGTCTCTGACC |
| | TGCACCGTGTCTGGCGGCTCTGTGTCCTCTGGCGATTACTACTGGACCTGGATCCGGCAGTCTC |
| | CTGGCAAAGGCCTGGAATGGATCGGCCACATCTACTACTCCGGCAACACCAACTACAACCCCA |
| | GCCTGAAGTCCCGGCTGACCATCTCCATCGACACCAGCAAGACCCAGTTCTCCCTGAAGCTGT |
| | CCTCTGTGACCGCCGCTGATACCGCCATCTACTATTGCGTGCGGGACAGAGTGACCGGCGCCT |
| | TTGATATTTGGGGCCAGGGCACCATGGTCACCGTGTCCAGTGCTTCTACCAAGGGACCCAGCG |
| | TGTTCCCTCTGGCTCCTTGCTCCAGATCCACCTCCGAGTCTACAGCTGCTCTGGGCTGTCTGGT |
| | CAAGGACTACTTCCCTGAGCCTGTGACCGTGTCCTGGAATTCTGGCGCTCTGACATCTGGCGT |
| | GCACACCTTTCCAGCTGTGCTGCAGTCTAGCGGCCTGTACTCTCTGTCCTCCGTCGTGACCGTG |
| | CCTTCCTCTAACTTTGGCACCCAGACCTACACCTGTAATGTGGACCACAAGCCATCCAACACCA |
| | AGGTGGACAAGACCGTGGAACGGAAGTGCTGCGTGGAATGCCCTCCTTGTCCTGCTCCTCCTG |
| | TGGCTGGCCCTTCCGTGTTTCTGTTCCCTCCAAAGCCTAAGGACACCCTGATGATCTCTCGGAC |
| | CCCTGAAGTGACCTGCGTGGTGGTGGATGTGTCTCACGAGGATCCCGAGGTGCAGTTCAATT |
| | GGTACGTGGACGGCGTGGAAGTGCACAACGCCAAGACCAAGCCTAGAGAGGAACAGTTCAA |
| | CTCCACCTTCAGAGTGGTGTCCGTGCTGACCGTGGTGCATCAGGATTGGCTGAACGGCAAAG |
| | AGTACAAGTGCAAGGTGTCCAACAAGGGCCTGCCTGCTCCTATCGAAAAGACCATCTCTAAGA |
| | CCAAGGGGCAGCCCCGGGAACCTCAGGTTTACACACTGCCTCCAAGCCGGGAAGAGATGACC |
| | AAGAACCAGGTGTCCCTGACCTGTCTCGTGAAGGGCTTCTACCCCTCCGATATCGCCGTGGAA |
| | TGGGAGTCTAATGGCCAGCCTGAGAACAACTACAAGACCACACCTCCTATGCTGGACTCCGAC |
| | GGCTCATTCTTCCTGTACTCCAAGCTGACAGTGGACAAGTCCAGATGGCAGCAGGGCAACGTG |
| | TTCTCCTGCTCCGTGATGCACGAGGCCCTGCACAATCACTACACCCAGAAGTCCCTGTCTCTGT |
| | CCCCTGGAAAAGGCGGCGGAGGATCTGGCGGAGGCGGTAGCGGTGGTGGCGGATCTGAAGT |
| | TCAGCTGGTTGAATCTGGCGGCGGACTGGTTCAACCAGGCGGATCTCTGAGACTGTCTTGTGC |
| | CGCCTCCGGCTTCACCTTCTCCTCTTACGCTATGTCCTGGGTCCGACAGGCCCCAGGCAAAGGA |
| | TTGGAGTGGGTGTCCCAGATCTCTCCCGCTGGCGGCTACACCAATTACGCCGACTCTGTGAAG |
| | GGCAGATTCACCATCTCTGCCGACACCTCCAAGAACACCGCCTACCTGCAGATGAACTCCCTGA |
| | GAGCCGAGGACACCGCCGTGTACTATTGTGCTAGAGGCGAGCTGCCATTCTTCCGGATGTCCC |
| | AAGTGATGGACGTGTGGGGACAGGGAACCCTCGTGACAGTTTCTAGCGGTGGCGGAGGTAG |
| | CGGAGGCGGTGGAAGCGGCGGAGGCGGAAGTGATATTCAGATGACCCAGTCTCCTTCCAGCC |
| | TGTCCGCTTCTGTGGGCGATAGAGTGACCATCACCTGTCGGGCCTCTCAGTTCCTGTCTAGTTA |
| | TCTGGCATGGTATCAGCAGAAGCCCGGCAAGGCTCCCAAGCTGTTGATCTACGGCGCCTCTGC |
| | TAGAGCTTCCGGCGTGCCATCTAGATTCTCCGGCTCTGGCTCTGGCACCGACTTTACCCTGACA |
| | ATCTCCAGCCTGCAGCCTGAGGACTTCGCCACCTACTACTGCCAGCAGTACCTGGCCTCTCCTG |
| | CCACATTTGGCCAGGGAACAAAGGTCGAGATCAAGCGCTGA |
| 19 | CAGGTTCAGCTGCAAGAGTCTGGCCCTGGCCTGGTCAAGCCTTCCGAAACACTGTCTCTGACC |
| | TGCACCGTGTCTGGCGGCTCTGTGTCCTCTGGCGATTACTACTGGACCTGGATCCGGCAGTCTC |
| | CTGGCAAAGGCCTGGAATGGATCGGCCACATCTACTACTCCGGCAACACCAACTACAACCCCA |
| | GCCTGAAGTCCCGGCTGACCATCTCCATCGACACCAGCAAGACCCAGTTCTCCCTGAAGCTGT |
| | CCTCTGTGACCGCCGCTGATACCGCCATCTACTATTGCGTGCGGGACAGAGTGACCGGCGCCT |
| | TTGATATTTGGGGCCAGGGCACCATGGTCACCGTGTCCAGTGCTTCTACCAAGGGACCCAGCG |
| | TGTTCCCTCTGGCTCCTTGCTCCAGATCCACCTCCGAGTCTACAGCTGCTCTGGGCTGTCTGGT |
| | CAAGGACTACTTCCCTGAGCCTGTGACCGTGTCCTGGAATTCTGGCGCTCTGACATCTGGCGT |
| | GCACACCTTTCCAGCTGTGCTGCAGTCTAGCGGCCTGTACTCTCTGTCCTCCGTCGTGACCGTG |
| | CCTTCCTCTAACTTTGGCACCCAGACCTACACCTGTAATGTGGACCACAAGCCATCCAACACCA |
| | AGGTGGACAAGACCGTGGAACGGAAGTGCTGCGTGGAATGCCCTCCTTGTCCTGCTCCTCCTG |
| | TGGCTGGCCCTTCCGTGTTTCTGTTCCCTCCAAAGCCTAAGGACACCCTGATGATCTCTCGGAC |
| | CCCTGAAGTGACCTGCGTGGTGGTGGATGTGTCTCACGAGGATCCCGAGGTGCAGTTCAATT |
| | GGTACGTGGACGGCGTGGAAGTGCACAACGCCAAGACCAAGCCTAGAGAGGAACAGTTCAA |
| | CTCCACCTTCAGAGTGGTGTCCGTGCTGACCGTGGTGCATCAGGATTGGCTGAACGGCAAAG |
| | AGTACAAGTGCAAGGTGTCCAACAAGGGCCTGCCTGCTCCTATCGAAAAGACCATCTCTAAGA |
| | CCAAGGGGCAGCCCCGGGAACCTCAGGTTTACACACTGCCTCCAAGCCGGGAAGAGATGACC |
| | AAGAACCAGGTGTCCCTGACCTGTCTCGTGAAGGGCTTCTACCCCTCCGATATCGCCGTGGAA |
| | TGGGAGTCTAATGGCCAGCCTGAGAACAACTACAAGACCACACCTCCTATGCTGGACTCCGAC |
| | GGCTCATTCTTCCTGTACTCCAAGCTGACAGTGGACAAGTCCAGATGGCAGCAGGGCAACGTG |
| | TTCTCCTGCTCCGTGATGCACGAGGCCCTGCACAATCACTACACCCAGAAGTCCCTGTCTCTGT |
| | CCCCTGGAAAAGGCGGCGGAGGATCTGGCGGAGGCGGTAGCGGTGGTGGCGGATCTGAAGT |
| | TCAGCTGGTTGAATCTGGCGGCGGACTGGTTCAACCAGGCGGATCTCTGAGACTGTCTTGTGC |
| | CGCCTCCGGCTTCACCTTCTCCTCTTACGCTATGTCCTGGGTCCGACAGGCCCCAGGCAAAGGA |
| | TTGGAGTGGGTGTCCCAGATCTCTCCCGCTGGCGGCTACACCAATTACGCCGACTCTGTGAAG |
| | GGCAGATTCACCATCTCTGCCGACACCTCCAAGAACACCGCCTACCTGCAGATGAACTCCCTGA |
| | GAGCCGAGGACACCGCCGTGTACTATTGTGCTAGAGGCGAGCTGCCCTACTTCCAGATGTCCA |
| | AAGTGATGGACGTGTGGGGACAGGGAACCCTCGTGACAGTTTCTAGCGGTGGCGGAGGTAG |
| | CGGAGGCGGTGGAAGCGGCGGAGGCGGAAGTGATATTCAGATGACCCAGTCTCCTTCCAGCC |
| | TGTCCGCTTCTGTGGGCGATAGAGTGACCATCACCTGTCGGGCCTCTCAGTACTTCTACTCCTA |
| | TCTGGCTTGGTATCAGCAGAAGCCTGGCAAGGCCCCTAAGCTGCTGATCTACGGCGCTTCTGC |
| | TAGAGCTTCCGGCGTGCCCTCCAGATTTTCTGGCTCTGGATCTGGCACCGACTTTACCCTGACA |
| | ATCTCCAGCCTGCAGCCTGAGGACTTCGCCACCTACTACTGCCAGCAGTACCTGGCCTCTCCTG |
| | CCACATTTGGCCAGGGAACAAAGGTCGAGATCAAGCGCTGA |
| 20 | CAGGTTCAGCTGCAAGAGTCTGGCCCTGGCCTGGTCAAGCCTTCCGAAACACTGTCTCTGACC |
| | TGCACCGTGTCTGGCGGCTCTGTGTCCTCTGGCGATTACTACTGGACCTGGATCCGGCAGTCTC |
| | CTGGCAAAGGCCTGGAATGGATCGGCCACATCTACTACTCCGGCAACACCAACTACAACCCCA |
| | GCCTGAAGTCCCGGCTGACCATCTCCATCGACACCAGCAAGACCCAGTTCTCCCTGAAGCTGT |
| | CCTCTGTGACCGCCGCTGATACCGCCATCTACTATTGCGTGCGGGACAGAGTGACCGGCGCCT |
| | TTGATATTTGGGGCCAGGGCACCATGGTCACCGTGTCCAGTGCTTCTACCAAGGGACCCAGCG |
| | TGTTCCCTCTGGCTCCTTCCAGCAAGTCTACCTCTGGCGGAACAGCTGCTCTGGGCTGTCTGGT |
| | CAAGGACTACTTCCCTGAGCCTGTGACCGTGTCCTGGAATTCTGGCGCTCTGACATCTGGCGT |
| | GCACACCTTTCCAGCTGTGCTGCAGTCTAGCGGCCTGTACTCTCTGTCCTCCGTCGTGACCGTG |
| | CCTTCTAGCTCTCTGGGCACCCAGACCTACATCTGCAATGTGAACCACAAGCCATCCAACACCA |
| | AGGTGGACAAGAAGGTGGAACCCAAGTCCTGCGACAAGACCCACACCTGTCCTCCATGTCCTG |
| | CTCCAGAACTGCTCGGCGGACCTTCCGTGTTCCTGTTTCCTCCAAAGCCTAAGGACACCCTGAT |
| | GATCTCTCGGACCCCTGAAGTGACCTGCGTGGTGGTGGATGTGTCTCACGAGGATCCCGAAGT |
| | GAAGTTCAATTGGTACGTGGACGGCGTGGAAGTGCACAACGCCAAGACCAAGCCTAGAGAG |
| | GAACAGTACAACTCCACCTACAGAGTGGTGTCCGTGCTGACCGTGCTGCACCAGGATTGGCTG |
| | AACGGCAAAGAGTACAAGTGCAAGGTGTCCAACAAGGCCCTGCCTGCTCCTATCGAAAAGAC |
| | CATCAGCAAGGCTAAGGGCCAGCCTCGGGAACCCCAGGTTTACACATTGCCTCCATCTCGGGA |
| | CGAGCTGACCAAGAACCAGGTGTCCCTGACCTGTCTCGTGAAGGGCTTCTACCCCTCCGATAT |
| | CGCCGTGGAATGGGAGTCCAATGGCCAGCCTGAGAACAACTACAAGACAACCCCTCCTGTGCT |
| | GGACTCCGACGGCTCATTCTTCCTGTACTCCAAGCTGACAGTGGACAAGTCCAGATGGCAGCA |
| | GGGCAACGTGTTCTCCTGCTCCGTGATGCACGAGGCCCTGCACAATCACTACACCCAGAAGTC |
| | CCTGTCTCTGTCCCCTGGAAAAGGCGGCGGAGGATCTGGCGGAGGTGGAAGCGGAGGCGGT |
| | GGATCTGAAGTGCAGCTGGTTGAAAGTGGCGGCGGATTGGTTCAGCCTGGCGGATCTCTGAG |
| | ACTGTCTTGTGCCGCCTCCGGCTTTACCTTCTCCTCCTACGCTATGTCCTGGGTCCGACAGGCTC |
| | CCGGAAAAGGACTTGAATGGGTGTCCCAGATCTCCCCTGCTGGCGGCTACACCAATTACGCCG |
| | ACTCTGTGAAGGGCAGATTCACCATCTCTGCCGACACCTCCAAGAACACCGCCTACCTGCAGA |
| | TGAACTCCCTGAGAGCCGAGGACACCGCCGTGTACTATTGTGCTAGAGGCGAGCTGCCCTACT |
| | TCCGGATGTCCAAAGTGATGGACGTGTGGGGACAGGGAACCCTCGTGACAGTTTCTAGTGGT |
| | GGCGGAGGAAGCGGCGGAGGCGGTTCTGGCGGTGGTGGATCTGATATCCAGATGACCCAGT |
| | CTCCTAGCAGCCTGTCTGCCTCTGTGGGCGATAGAGTGACCATCACCTGTCGGGCCTCTCAGTT |
| | CCTGTCCAGCTACCTGGCTTGGTATCAGCAGAAGCCTGGCAAGGCCCCTAAGCTGCTGATCTA |
| | CGGCGCTTCTGCTAGAGCTTCCGGCGTGCCCTCCAGATTTTCTGGCTCTGGATCTGGCACCGAC |
| | TTTACCCTGACAATCTCCAGCCTGCAGCCTGAGGACTTCGCCACCTACTACTGCCAGCAGTACC |
| | TGGCCTCTCCTGCCACATTTGGCCAGGGAACAAAGGTCGAGATCAAGCGCTGA |
| 21 | CAGGTTCAGCTGCAAGAGTCTGGCCCTGGCCTGGTCAAGCCTTCCGAAACACTGTCTCTGACC |
| | TGCACCGTGTCTGGCGGCTCTGTGTCCTCTGGCGATTACTACTGGACCTGGATCCGGCAGTCTC |
| | CTGGCAAAGGCCTGGAATGGATCGGCCACATCTACTACTCCGGCAACACCAACTACAACCCCA |
| | GCCTGAAGTCCCGGCTGACCATCTCCATCGACACCAGCAAGACCCAGTTCTCCCTGAAGCTGT |
| | CCTCTGTGACCGCCGCTGATACCGCCATCTACTATTGCGTGCGGGACAGAGTGACCGGCGCCT |
| | TTGATATTTGGGGCCAGGGCACCATGGTCACCGTGTCCAGTGCTTCTACCAAGGGACCCAGCG |
| | TGTTCCCTCTGGCTCCTTCCAGCAAGTCTACCTCTGGCGGAACAGCTGCTCTGGGCTGTCTGGT |
| | CAAGGACTACTTCCCTGAGCCTGTGACCGTGTCCTGGAATTCTGGCGCTCTGACATCTGGCGT |
| | GCACACCTTTCCAGCTGTGCTGCAGTCTAGCGGCCTGTACTCTCTGTCCTCCGTCGTGACCGTG |
| | CCTTCTAGCTCTCTGGGCACCCAGACCTACATCTGCAATGTGAACCACAAGCCATCCAACACCA |
| | AGGTGGACAAGAAGGTGGAACCCAAGTCCTGCGACAAGACCCACACCTGTCCTCCATGTCCTG |
| | CTCCAGAACTGCTCGGCGGACCTTCCGTGTTCCTGTTTCCTCCAAAGCCTAAGGACACCCTGAT |
| | GATCTCTCGGACCCCTGAAGTGACCTGCGTGGTGGTGGATGTGTCTCACGAGGATCCCGAAGT |
| | GAAGTTCAATTGGTACGTGGACGGCGTGGAAGTGCACAACGCCAAGACCAAGCCTAGAGAG |
| | GAACAGTACAACTCCACCTACAGAGTGGTGTCCGTGCTGACCGTGCTGCACCAGGATTGGCTG |
| | AACGGCAAAGAGTACAAGTGCAAGGTGTCCAACAAGGCCCTGCCTGCTCCTATCGAAAAGAC |
| | CATCAGCAAGGCTAAGGGCCAGCCTCGGGAACCCCAGGTTTACACATTGCCTCCATCTCGGGA |
| | CGAGCTGACCAAGAACCAGGTGTCCCTGACCTGTCTCGTGAAGGGCTTCTACCCCTCCGATAT |
| | CGCCGTGGAATGGGAGTCCAATGGCCAGCCTGAGAACAACTACAAGACAACCCCTCCTGTGCT |
| | GGACTCCGACGGCTCATTCTTCCTGTACTCCAAGCTGACAGTGGACAAGTCCAGATGGCAGCA |
| | GGGCAACGTGTTCTCCTGCTCCGTGATGCACGAGGCCCTGCACAATCACTACACCCAGAAGTC |
| | CCTGTCTCTGTCCCCTGGAAAAGGCGGCGGAGGATCTGGCGGAGGTGGAAGCGGAGGCGGT |
| | GGATCTGAAGTGCAGCTGGTTGAAAGTGGCGGCGGATTGGTTCAGCCTGGCGGATCTCTGAG |
| | ACTGTCTTGTGCCGCCTCCGGCTTTACCTTCTCCTCCTACGCTATGTCCTGGGTCCGACAGGCTC |
| | CCGGAAAAGGACTTGAATGGGTGTCCCAGATCTCCCCTGCTGGCGGCTACACCAATTACGCCG |
| | ACTCTGTGAAGGGCAGATTCACCATCTCTGCCGACACCTCCAAGAACACCGCCTACCTGCAGA |
| | TGAACTCCCTGAGAGCCGAGGACACCGCCGTGTACTATTGTGCTAGAGGCGAGCTGCCCTACT |
| | TCCGGATGTCCAAAGTGATGGACGTGTGGGGACAGGGAACCCTCGTGACAGTGTCTAGCGGA |
| | GATGGATCTAGTGGTGGCGGAGGCGCTTCCGACATCCAGATGACACAGTCTCCCTCCAGCCTG |
| | TCTGCCTCTGTGGGCGATAGAGTGACCATCACCTGTCGGGCCTCTCAGTTCCTGTCCAGCTACC |
| | TGGCTTGGTATCAGCAGAAGCCTGGCAAGGCCCCTAAGCTGCTGATCTACGGCGCTTCTGCTA |
| | GAGCTTCCGGCGTGCCCTCCAGATTTTCTGGCTCTGGATCTGGCACCGACTTTACCCTGACAAT |
| | CAGCAGCCTGCAGCCTGAGGACTTCGCCACCTACTACTGCCAGCAGTACCTGGCCTCTCCTGCC |
| | ACATTTGGCCAGGGAACAAAGGTCGAGATCAAGTGA |
| 22 | CAGGTTCAGCTGCAAGAGTCTGGCCCTGGCCTGGTCAAGCCTTCCGAAACACTGTCTCTGACC |
| | TGCACCGTGTCTGGCGGCTCTGTGTCCTCTGGCGATTACTACTGGACCTGGATCCGGCAGTCTC |
| | CTGGCAAAGGCCTGGAATGGATCGGCCACATCTACTACTCCGGCAACACCAACTACAACCCCA |
| | GCCTGAAGTCCCGGCTGACCATCTCCATCGACACCAGCAAGACCCAGTTCTCCCTGAAGCTGT |
| | CCTCTGTGACCGCCGCTGATACCGCCATCTACTATTGCGTGCGGGACAGAGTGACCGGCGCCT |
| | TTGATATTTGGGGCCAGGGCACCATGGTCACCGTGTCCAGTGCTTCTACCAAGGGACCCAGCG |
| | TGTTCCCTCTGGCTCCTTCCAGCAAGTCTACCTCTGGCGGAACAGCTGCTCTGGGCTGTCTGGT |
| | CAAGGACTACTTCCCTGAGCCTGTGACCGTGTCCTGGAATTCTGGCGCTCTGACATCTGGCGT |
| | GCACACCTTTCCAGCTGTGCTGCAGTCTAGCGGCCTGTACTCTCTGTCCTCCGTCGTGACCGTG |
| | CCTTCTAGCTCTCTGGGCACCCAGACCTACATCTGCAATGTGAACCACAAGCCATCCAACACCA |
| | AGGTGGACAAGAAGGTGGAACCCAAGTCCTGCGACAAGACCCACACCTGTCCTCCATGTCCTG |
| | CTCCAGAACTGCTCGGCGGACCTTCCGTGTTCCTGTTTCCTCCAAAGCCTAAGGACACCCTGAT |
| | GATCTCTCGGACCCCTGAAGTGACCTGCGTGGTGGTGGATGTGTCTCACGAGGATCCCGAAGT |
| | GAAGTTCAATTGGTACGTGGACGGCGTGGAAGTGCACAACGCCAAGACCAAGCCTAGAGAG |
| | GAACAGTACAACTCCACCTACAGAGTGGTGTCCGTGCTGACCGTGCTGCACCAGGATTGGCTG |
| | AACGGCAAAGAGTACAAGTGCAAGGTGTCCAACAAGGCCCTGCCTGCTCCTATCGAAAAGAC |
| | CATCAGCAAGGCTAAGGGCCAGCCTCGGGAACCCCAGGTTTACACATTGCCTCCATCTCGGGA |
| | CGAGCTGACCAAGAACCAGGTGTCCCTGACCTGTCTCGTGAAGGGCTTCTACCCCTCCGATAT |
| | CGCCGTGGAATGGGAGTCCAATGGCCAGCCTGAGAACAACTACAAGACAACCCCTCCTGTGCT |
| | GGACTCCGACGGCTCATTCTTCCTGTACTCCAAGCTGACAGTGGACAAGTCCAGATGGCAGCA |
| | GGGCAACGTGTTCTCCTGCTCCGTGATGCACGAGGCCCTGCACAATCACTACACCCAGAAGTC |
| | CCTGTCTCTGTCCCCTGGAAAAGGCGGCGGAGGATCTGGCGGAGGTGGAAGCGGAGGCGGT |
| | GGATCTGAAGTGCAGCTGGTTGAAAGTGGCGGCGGATTGGTTCAGCCTGGCGGATCTCTGAG |
| | ACTGTCTTGTGCCGCCTCCGGCTTTACCTTCTCCTCCTACGCTATGTCCTGGGTCCGACAGGCTC |
| | CCGGAAAAGGACTTGAATGGGTGTCCCAGATCTCCCCTGCTGGCGGCTACACCAATTACGCCG |
| | ACTCTGTGAAGGGCAGATTCACCATCTCTGCCGACACCTCCAAGAACACCGCCTACCTGCAGA |
| | TGAACTCCCTGAGAGCCGAGGACACCGCCGTGTACTATTGTGCTAGAGGCGAGCTGCCCTACT |
| | TCCGGATGTCCAAAGTGATGGACGTGTGGGGACAGGGAACCCTCGTGACAGTTTCTAGTGGT |
| | GGCGGAGGAAGCGGCGGAGGCGGTTCTGGCGGTGGTGGCTCTGGCGGTGGCGGATCTGATA |
| | TCCAGATGACCCAGTCTCCTAGCAGCCTGTCTGCCTCTGTGGGCGATAGAGTGACCATCACCT |
| | GTCGGGCCTCTCAGTTCCTGTCCAGCTACCTGGCTTGGTATCAGCAGAAGCCTGGCAAGGCCC |
| | CTAAGCTGCTGATCTACGGCGCTTCTGCTAGAGCTTCCGGCGTGCCCTCCAGATTCTCTGGCTC |
| | TGGATCTGGCACCGACTTTACCCTGACAATCTCCAGCCTGCAGCCTGAGGACTTCGCCACCTAC |
| | TACTGCCAGCAGTACCTGGCCTCTCCTGCCACATTTGGCCAGGGAACAAAGGTCGAGATCAAG |
| | TGA |
| 23 | CAGGTTCAGCTGCAAGAGTCTGGCCCTGGCCTGGTCAAGCCTTCCGAAACACTGTCTCTGACC |
| | TGCACCGTGTCTGGCGGCTCTGTGTCCTCTGGCGATTACTACTGGACCTGGATCCGGCAGTCTC |
| | CTGGCAAAGGCCTGGAATGGATCGGCCACATCTACTACTCCGGCAACACCAACTACAACCCCA |
| | GCCTGAAGTCCCGGCTGACCATCTCCATCGACACCAGCAAGACCCAGTTCTCCCTGAAGCTGT |
| | CCTCTGTGACCGCCGCTGATACCGCCATCTACTATTGCGTGCGGGACAGAGTGACCGGCGCCT |
| | TTGATATTTGGGGCCAGGGCACCATGGTCACCGTGTCCAGTGCTTCTACCAAGGGACCCAGCG |
| | TGTTCCCTCTGGCTCCTTCCAGCAAGTCTACCTCTGGCGGAACAGCTGCTCTGGGCTGTCTGGT |
| | CAAGGACTACTTCCCTGAGCCTGTGACCGTGTCCTGGAATTCTGGCGCTCTGACATCTGGCGT |
| | GCACACCTTTCCAGCTGTGCTGCAGTCTAGCGGCCTGTACTCTCTGTCCTCCGTCGTGACCGTG |
| | CCTTCTAGCTCTCTGGGCACCCAGACCTACATCTGCAATGTGAACCACAAGCCATCCAACACCA |
| | AGGTGGACAAGAAGGTGGAACCCAAGTCCTGCGACAAGACCCACACCTGTCCTCCATGTCCTG |
| | CTCCAGAACTGCTCGGCGGACCTTCCGTGTTCCTGTTTCCTCCAAAGCCTAAGGACACCCTGAT |
| | GATCTCTCGGACCCCTGAAGTGACCTGCGTGGTGGTGGATGTGTCTCACGAGGATCCCGAAGT |
| | GAAGTTCAATTGGTACGTGGACGGCGTGGAAGTGCACAACGCCAAGACCAAGCCTAGAGAG |
| | GAACAGTACAACTCCACCTACAGAGTGGTGTCCGTGCTGACCGTGCTGCACCAGGATTGGCTG |
| | AACGGCAAAGAGTACAAGTGCAAGGTGTCCAACAAGGCCCTGCCTGCTCCTATCGAAAAGAC |
| | CATCAGCAAGGCTAAGGGCCAGCCTCGGGAACCCCAGGTTTACACATTGCCTCCATCTCGGGA |
| | CGAGCTGACCAAGAACCAGGTGTCCCTGACCTGTCTCGTGAAGGGCTTCTACCCCTCCGATAT |
| | CGCCGTGGAATGGGAGTCCAATGGCCAGCCTGAGAACAACTACAAGACAACCCCTCCTGTGCT |
| | GGACTCCGACGGCTCATTCTTCCTGTACTCCAAGCTGACAGTGGACAAGTCCAGATGGCAGCA |
| | GGGCAACGTGTTCTCCTGCTCCGTGATGCACGAGGCCCTGCACAATCACTACACCCAGAAGTC |
| | CCTGTCTCTGTCCCCTGGAAAAGGCGGCGGAGGATCTGGCGGAGGTGGAAGCGGAGGCGGT |
| | GGATCTGATATCCAGATGACCCAGTCTCCTAGCAGCCTGTCTGCCTCTGTGGGCGATAGAGTG |
| | ACCATCACCTGTCGGGCCTCTCAGTTCCTGTCCAGCTACCTGGCTTGGTATCAGCAGAAGCCTG |
| | GCAAGGCCCCTAAGCTGCTGATCTACGGCGCTTCTGCTAGAGCTTCCGGCGTGCCCTCCAGAT |
| | TTTCTGGCTCTGGATCTGGCACCGACTTTACCCTGACAATCTCCAGCCTGCAGCCTGAGGACTT |
| | CGCCACCTACTACTGCCAGCAGTACCTGGCCTCTCCTGCCACATTTGGCCAGGGAACAAAGGT |
| | GGAAATCAAAGGTGGCGGCGGTAGTGGTGGCGGAGGAAGCGGCGGAGGCGGCTCTGAAGT |
| | TCAGCTTGTTGAATCTGGCGGCGGACTGGTTCAGCCTGGCGGATCTCTGAGACTGTCTTGTGC |
| | CGCCAGCGGCTTCACCTTCTCCTCTTACGCTATGTCCTGGGTCCGACAGGCCCCAGGCAAAGG |
| | ATTGGAGTGGGTGTCCCAGATCTCTCCTGCTGGCGGCTACACCAATTACGCCGACTCTGTGAA |
| | GGGCAGATTCACCATCTCTGCCGACACCTCCAAGAACACCGCCTACCTGCAGATGAACTCCCT |
| | GAGAGCCGAGGACACCGCCGTGTACTATTGTGCTAGAGGCGAGCTGCCCTACTTCCGGATGT |
| | CCAAAGTGATGGACGTGTGGGGACAGGGAACCCTCGTGACAGTGTCCTCTTGA |
| 24 | CAGGTTCAGCTGCAAGAGTCTGGCCCTGGCCTGGTCAAGCCTTCCGAAACACTGTCTCTGACC |
| | TGCACCGTGTCTGGCGGCTCTGTGTCCTCTGGCGATTACTACTGGACCTGGATCCGGCAGTCTC |
| | CTGGCAAAGGCCTGGAATGGATCGGCCACATCTACTACTCCGGCAACACCAACTACAACCCCA |
| | GCCTGAAGTCCCGGCTGACCATCTCCATCGACACCAGCAAGACCCAGTTCTCCCTGAAGCTGT |
| | CCTCTGTGACCGCCGCTGATACCGCCATCTACTATTGCGTGCGGGACAGAGTGACCGGCGCCT |
| | TTGATATTTGGGGCCAGGGCACCATGGTCACCGTGTCCAGTGCTTCTACCAAGGGACCCAGCG |
| | TGTTCCCTCTGGCTCCTTCCAGCAAGTCTACCTCTGGCGGAACAGCTGCTCTGGGCTGTCTGGT |
| | CAAGGACTACTTCCCTGAGCCTGTGACCGTGTCCTGGAATTCTGGCGCTCTGACATCTGGCGT |
| | GCACACCTTTCCAGCTGTGCTGCAGTCTAGCGGCCTGTACTCTCTGTCCTCCGTCGTGACCGTG |
| | CCTTCTAGCTCTCTGGGCACCCAGACCTACATCTGCAATGTGAACCACAAGCCATCCAACACCA |
| | AGGTGGACAAGAAGGTGGAACCCAAGTCCTGCGACAAGACCCACACCTGTCCTCCATGTCCTG |
| | CTCCAGAACTGCTCGGCGGACCTTCCGTGTTCCTGTTTCCTCCAAAGCCTAAGGACACCCTGAT |
| | GATCTCTCGGACCCCTGAAGTGACCTGCGTGGTGGTGGATGTGTCTCACGAGGATCCCGAAGT |
| | GAAGTTCAATTGGTACGTGGACGGCGTGGAAGTGCACAACGCCAAGACCAAGCCTAGAGAG |
| | GAACAGTACAACTCCACCTACAGAGTGGTGTCCGTGCTGACCGTGCTGCACCAGGATTGGCTG |
| | AACGGCAAAGAGTACAAGTGCAAGGTGTCCAACAAGGCCCTGCCTGCTCCTATCGAAAAGAC |
| | CATCAGCAAGGCTAAGGGCCAGCCTCGGGAACCCCAGGTTTACACATTGCCTCCATCTCGGGA |
| | CGAGCTGACCAAGAACCAGGTGTCCCTGACCTGTCTCGTGAAGGGCTTCTACCCCTCCGATAT |
| | CGCCGTGGAATGGGAGTCCAATGGCCAGCCTGAGAACAACTACAAGACAACCCCTCCTGTGCT |
| | GGACTCCGACGGCTCATTCTTCCTGTACTCCAAGCTGACAGTGGACAAGTCCAGATGGCAGCA |
| | GGGCAACGTGTTCTCCTGCTCCGTGATGCACGAGGCCCTGCACAATCACTACACCCAGAAGTC |
| | CCTGTCTCTGTCCCCTGGAAAAGGCGGCGGAGGATCTGGCGGAGGTGGAAGCGGAGGCGGT |
| | GGATCTGAAGTGCAGCTGTTGGAAAGTGGCGGCGGATTGGTTCAGCCTGGCGGATCTCTGAG |
| | ACTGTCTTGTGCCGCCTCCGGCTTTACCTTCTCCTCCTACGCTATGTCCTGGGTCCGACAGGCTC |
| | CCGGAAAAGGACTTGAATGGGTGTCCCAGATCTCCCCTGCTGGCGGCTACACCAATTACGCCG |
| | ACTCTGTGAAGGGCAGATTCACCATCTCTCGGGACAACTCCAAGAACACCCTGTACCTGCAGA |
| | TGAACTCCCTGAGAGCCGAGGACACCGCCGTGTACTATTGTGCTAGAGGCGAGCTGCCCTACT |
| | TCCGGATGTCCAAAGTGATGGACGTGTGGGGACAGGGAACCCTCGTGACAGTTTCTAGTGGT |
| | GGCGGAGGAAGCGGCGGAGGCGGTTCTGGCGGTGGCGGATCTGAAATTGTGCTGACCCAGT |
| | CTCCAGGCACACTCAGTTTGAGCCCTGGCGAGAGAGCTACCCTGAGCTGTAGAGCCTCTCAGT |
| | TCCTGTCCAGCTACCTGGCTTGGTATCAGCAGAAGCCAGGACAGGCCCCTCGGCTGTTGATCT |
| | ATGGCGCTTCTGCTAGAGCCAGCGGCATCCCTGATAGATTCTCCGGCTCTGGCTCTGGCACCG |
| | ACTTCACCCTGACAATCTCCCGGCTGGAACCTGAGGACTTCGCTGTGTACTACTGCCAGCAGTA |
| | CCTGGCCTCTCCTGCCACATTTGGCCAGGGAACAAAGGTCGAGATCAAGTGA |
| 25 | GACATCCAGATGACCCAGTCTCCATCATCCCTGTCGGCCTCAGTGGGCGACAGAGTGACCATC |
| | ACTTGTCAAGCCTCCCAAGACATTAGCAACTACCTGAACTGGTACCAGCAGAAGCCCGGAAAG |
| | GCCCCGAAGCTGCTCATCTATGACGCTTCCAACCTTGAGACTGGAGTGCCTTCGCGCTTCTCCG |
| | GCTCCGGGAGCGGTACCGATTTCACCTTCACCATCTCCTCCCTGCAACCCGAGGACATTGCGAC |
| | TTACTTCTGCCAACATTTCGATCACCTCCCTCTCGCGTTCGGCGGCGGAACTAAGGTCGAGATT |
| | AAGCGGACCGTGGCTGCCCCGTCCGTGTTCATCTTCCCGCCGTCCGATGAACAGCTGAAGTCC |
| | GGTACCGCATCAGTCGTGTGCTTGCTGAACAACTTCTACCCCCGGGAAGCCAAGGTCCAGTGG |
| | AAAGTGGACAATGCGCTGCAGTCGGGAAACTCGCAGGAATCCGTGACCGAACAGGATTCGAA |
| | GGACAGCACATACAGCCTGTCATCCACCCTCACGCTGTCGAAGGCCGACTACGAGAAGCACAA |
| | AGTGTACGCCTGCGAAGTGACCCACCAAGGGCTTAGCAGCCCTGTGACCAAGTCCTTCAACCG |
| | CGGAGAGTGC |
| 26 | GGCGGTGGCGGATCC |
| 27 | GGCGGCGGGGGCAGC |
| 28 | GGCGGCGGAGGATCT |
| 29 | GGCGGAGGCGGTAGC |
| 30 | GGTGGTGGCGGATCT |
| 31 | GGCGGCGGAGGATCT |
| 32 | GGCGGAGGTGGAAGC |
| 33 | GGAGGCGGTGGATCT |
| 34 | GGTGGCGGAGGAAGC |
| 35 | GGCGGAGGCGGTTCT |
| 36 | GGCGGTGGCGGATCT |
| 37 | GGCGGTGGCGGATCC |
| 38 | GGCGGCGGGGGCAGC |
| 39 | QVQLQESGPGLVKPSETLSLTCTVSGGSVSSGDYYWTWIRQSPGKGLEWIGHIY |
| | YSGNTNYNPSLKSRLTISIDTSKTQFSLKLSSVTAADTAIYYCVRDRVTGAFDIW |
| | GQGTMVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHED |
| | PEVKFNWYVDGVEVHNAKTKPREEQYNSTYRVVSVLTVLHQDWLNGKEYKC |
| | KVSNKALPAPIEKTISKAKGQPREPQVYTLPPSRDELTKNQVSLTCLVKGFYPSD |
| | IAVEWESNGQPENNYKTTPPVLDSDGSFFLYSKLTVDKSRWQQGNVFSCSVMH |
| | EALHNHYTQKSLSLSPGKGGGGSGGGGSGGGGSEVQLLESGGGLVQPGGSLRL |
| | SCAASGFTFSSYAMSWVRQAPGKGLEWVSQISPAGGYTNYADSVKGRFTISRD |
| | NSKNTLYLQMNSLRAEDTAVYYCARGELPYHRMSKVMDVWGQGTLVTVSSG |
| | GGGSGGGGSGGGGSEIVLTQSPGTLSLSPGERATLSCRASQFLSSYLAWYQQKP |
| | GQAPRLLIYGASARASGIPDRESGSGSGTDFTLTISRLEPEDFAVYYCQQYLASP |
| | ATFGQGTKVEIK |
| 40 | CAGGTTCAGCTGCAAGAGTCTGGCCCTGGCCTGGTCAAGCCTTCCGAAACA |
| | CTGTCTCTGACCTGCACCGTGTCTGGCGGCTCTGTGTCCTCTGGCGATTACTA |
| | CTGGACCTGGATCCGGCAGTCTCCTGGCAAAGGCCTGGAATGGATCGGCCA |
| | CATCTACTACTCCGGCAACACCAACTACAACCCCAGCCTGAAGTCCCGGCTG |
| | ACCATCTCCATCGACACCAGCAAGACCCAGTTCTCCCTGAAGCTGTCCTCTG |
| | TGACCGCCGCTGATACCGCCATCTACTATTGCGTGCGGGACAGAGTGACCG |
| | GCGCCTTTGATATTTGGGGCCAGGGCACCATGGTCACCGTGTCCAGTGCTTC |
| | TACCAAGGGACCCAGCGTGTTCCCTCTGGCTCCTTCCAGCAAGTCTACCTCT |
| | GGCGGAACAGCTGCTCTGGGCTGTCTGGTCAAGGACTACTTCCCTGAGCCTG |
| | TGACCGTGTCCTGGAATTCTGGCGCTCTGACATCTGGCGTGCACACCTTTCC |
| | AGCTGTGCTGCAGTCTAGCGGCCTGTACTCTCTGTCCTCCGTCGTGACCGTG |
| | CCTTCTAGCTCTCTGGGCACCCAGACCTACATCTGCAATGTGAACCACAAGC |
| | CATCCAACACCAAGGTGGACAAGAAGGTGGAACCCAAGTCCTGCGACAAG |
| | ACCCACACCTGTCCTCCATGTCCTGCTCCAGAACTGCTCGGCGGACCTTCCG |
| | TGTTCCTGTTTCCTCCAAAGCCTAAGGACACCCTGATGATCTCTCGGACCCC |
| | TGAAGTGACCTGCGTGGTGGTGGATGTGTCTCACGAGGATCCCGAAGTGAA |
| | GTTCAATTGGTACGTGGACGGCGTGGAAGTGCACAACGCCAAGACCAAGCC |
| | TAGAGAGGAACAGTACAACTCCACCTACAGAGTGGTGTCCGTGCTGACCGT |
| | GCTGCACCAGGATTGGCTGAACGGCAAAGAGTACAAGTGCAAGGTGTCCAA |
| | CAAGGCCCTGCCTGCTCCTATCGAAAAGACCATCAGCAAGGCTAAGGGCCA |
| | GCCTCGGGAACCCCAGGTTTACACATTGCCTCCATCTCGGGACGAGCTGACC |
| | AAGAACCAGGTGTCCCTGACCTGTCTCGTGAAGGGCTTCTACCCCTCCGATA |
| | TCGCCGTGGAATGGGAGTCCAATGGCCAGCCTGAGAACAACTACAAGACAA |
| | CCCCTCCTGTGCTGGACTCCGACGGCTCATTCTTCCTGTACTCCAAGCTGAC |
| | AGTGGACAAGTCCAGATGGCAGCAGGGCAACGTGTTCTCCTGCTCCGTGAT |
| | GCACGAGGCCCTGCACAATCACTACACCCAGAAGTCCCTGTCTCTGTCCCCT |
| | GGAAAAGGCGGCGGAGGATCTGGCGGAGGTGGAAGCGGAGGCGGTGGATC |
| | TGAAGTGCAGCTGTTGGAAAGTGGCGGCGGATTGGTTCAGCCTGGCGGATC |
| | TCTGAGACTGTCTTGTGCCGCCTCCGGCTTTACCTTCTCCTCCTACGCTATGT |
| | CCTGGGTCCGACAGGCTCCCGGAAAAGGACTTGAATGGGTGTCCCAGATCT |
| | CCCCTGCTGGCGGCTACACCAATTACGCCGACTCTGTGAAGGGCAGATTCAC |
| | CATCTCTCGGGACAACTCCAAGAACACCCTGTACCTGCAGATGAACTCCCTG |
| | AGAGCCGAGGACACCGCCGTGTACTATTGTGCTAGAGGCGAGCTGCCCTAC |
| | CACCGGATGTCCAAAGTGATGGATGTGTGGGGACAGGGAACCCTCGTGACA |
| | GTCTCTAGTGGTGGCGGAGGAAGCGGCGGAGGCGGTTCTGGCGGTGGCGGA |
| | TCTGAAATTGTGCTGACCCAGTCTCCAGGCACACTCAGTTTGAGCCCTGGCG |
| | AGAGAGCTACCCTGAGCTGTAGAGCCTCTCAGTTCCTGTCCAGCTACCTGGC |
| | TTGGTATCAGCAGAAGCCAGGACAGGCCCCTCGGCTGTTGATCTATGGCGCT |
| | TCTGCTAGAGCCAGCGGCATCCCTGATAGATTCTCCGGCTCTGGCTCTGGCA |
| | CCGACTTCACCCTGACAATCTCCCGGCTGGAACCTGAGGACTTCGCTGTGTA |
| | CTACTGCCAGCAGTACCTGGCCTCTCCTGCCACATTTGGCCAGGGAACAAAG |
| | GTCGAGATCAAG |
| 41 | EVQLVESGGGLVQPGGSLRLSCAASGFTFSSYAMSWVRQAPGKGLEWVSQISPAGGYTNYADSV |
| | KGRFTISADTSKNTAYLQMNSLRAEDTAVYYCARGELPYFRMSKVMDVWGQGTLVTVSSASTKG |
| | PSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPELLGGPSVFLFPPKPKDTLMISRTP |
| | EVTCVVVDVSHEDPEVKFNWYVDGVEVHN30AKTKPREEQYNSTYRVVSVLTVLHQDWLNGKEY |
| | KCKVSNKALPAPIEKTISKAKGQPREPQVYTLPPSREEMTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGK |
| 42 | EVQLVESGGGLVQPGGSLRLSCAASGFTFSSYAMSWVRQAPGKGLEWVSQISPAGGYTNYADSV |
| | KGRFTISADTSKNTAYLQMNSLRAEDTAVYYCARGELPYYQMSKVMDVWGQGTLVTVSSASTKG |
| | PSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK |
| 43 | EVQLVESGGGLVQPGGSLRLSCAASGFTFSSYAMSWVRQAPGKGLEWVSQISPAGGYTNYADSV |
| | KGRFTISADTSKNTAYLQMNSLRAEDTAVYYCARGELPFFRMSQVMDVWGQGTLVTVSSASTKG |
| | PSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK |
| 44 | EVQLVESGGGLVQPGGSLRLSCAASGFTFSSYAMSWVRQAPGKGLEWVSQISPAGGYTNYADSV |
| | KGRFTISADTSKNTAYLQMNSLRAEDTAVYYCARGELPYFRMSKVMDVWGQGTLVTVSSASTKG |
| | PSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK |
| 45 | EVQLLESGGGLVQPGGSLRLSCAASGFTFSSYAMSWVRQAPGKGLEWVSQISPAGGYTNYADSV |
| | KGRFTISRDNSKNTLYLQMNSLRAEDTAVYYCARGELPYFRMSKVMDVWGQGTLVTVSSASTKG |
| | PSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK |
| 46 | EVQLLESGGGLVQPGGSLRLSCAASGFTFSSYAMSWVRQAPGKGLEWVSQISPAGGYTNYADSV |
| | KGRFTISRDNSKNTLYLQMNSLRAEDTAVYYCARGELPYYQMSKVMDVWGQGTLVTVSSASTKG |
| | PSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK |
| 47 | EVQLLESGGGLVQPGGSLRLSCAASGFTFSSYAMSWVRQAPGKGLEWVSQISPAGGYTNYADSV |
| | KGRFTISRDNSKNTLYLQMNSLRAEDTAVYYCARGELPFFRMSQVMDVWGQGTLVTVSSASTKG |
| | PSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK |
| 48 | DIQMTQSPSSLSASVGDRVTITCRASQFLSSYLAWYQQKPGKAPKLLIYGASAR |
| | ASGVPSRFSGSGSGTDFTLTISSLQPEDFATYYCQQYLASPATFGQGTKVEIKRT |
| | VAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| 49 | DIQMTQSPSSLSASVGDRVTITCRASQYFSSYLAWYQQKPGKAPKLLIYGASAR |
| | ASGVPSRFSGSGSGTDFTLTISSLQPEDFATYYCQQYLASPATFGQGTKVEIKRT |
| | VAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| 50 | DIQMTQSPSSLSASVGDRVTITCRASQFLSSYLAWYQQKPGKAPKLLIYGASAR |
| | ASGVPSRFSGSGSGTDFTLTISSLQPEDFATYYCQQYLASPATFGQGTKVEIKRT |
| | VAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSENRGEC |
| 51 | DIQMTQSPSSLSASVGDRVTITCRASQFLSSFLAWYQQKPGKAPKLLIYGASAR |
| | ASGVPSRFSGSGSGTDFTLTISSLQPEDFATYYCQQYLASPATFGQGTKVEIKRT |
| | VAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| 52 | EIVLTQSPGTLSLSPGERATLSCRASQFLSSYLAWYQQKPGQAPRLLIYGASARA |
| | SGIPDRFSGSGSGTDFTLTISRLEPEDFAVYYCQQYLASPATFGQGTKVEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 53 | EIVLTQSPGTLSLSPGERATLSCRASQFLSSYLAWYQQKPGQAPRLLIYGASARA |
| | SGIPDRFSGSGSGTDFTLTISRLEPEDFAVYYCQQYLASPATFGQGTKVEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 54 | EIVLTQSPGTLSLSPGERATLSCRASQFLSSYLAWYQQKPGQAPRLLIYGASARA |
| | SGIPDRFSGSGSGTDFTLTISRLEPEDFAVYYCQQYLASPATFGQGTKVEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 55 | GAGGTGCAGCTGGTGGAGAGCGGCGGCGGCCTGGTGCAGCCCGGCGGCAG |
| | CCTGAGGCTGAGCTGCGCCGCCAGCGGCTTCACCTTCAGCAGCTACGCCATG |
| | AGCTGGGTGAGGCAGGCCCCCGGCAAGGGCCTGGAGTGGGTGAGCCAGATC |
| | AGCCCCGCCGGCGGCTACACCAACTACGCCGACAGCGTGAAGGGCAGGTTC |
| | ACCATCAGCGCCGACACCAGCAAGAACACCGCCTACCTGCAGATGAACAGC |
| | CTGAGGGCCGAGGACACCGCCGTGTACTACTGCGCCAGGGGCGAGCTGCCC |
| | TACTTCAGGATGAGCAAGGTGATGGACGTGTGGGGCCAGGGCACCCTGGTG |
| | ACCGTGAGCAGCGCCAGCACCAAGGGCCCCAGCGTGTTCCCCCTGGCCCCC |
| | AGCAGCAAGAGCACCAGCGGCGGCACCGCCGCCCTGGGCTGCCTGGTGAAG |
| | GACTACTTCCCCGAGCCCGTGACCGTGAGCTGGAACAGCGGCGCCCTGACC |
| | AGCGGCGTGCACACCTTCCCCGCCGTGCTGCAGAGCAGCGGCCTGTACAGC |
| | CTGAGCAGCGTGGTGACCGTGCCCAGCAGCAGCCTGGGCACCCAGACCTAC |
| | ATCTGCAACGTGAACCACAAGCCCAGCAACACCAAGGTGGACAAGAAGGT |
| | GGAGCCCAAGAGCTGCGACAAGACCCACACCTGCCCCCCCTGCCCCGCCCC |
| | CGAGCTGCTGGGCGGCCCCAGCGTGTTCCTGTTCCCCCCCAAGCCCAAGGAC |
| | ACCCTGATGATCAGCAGGACCCCCGAGGTGACCTGCGTGGTGGTGGACGTG |
| | AGCCACGAGGACCCCGAGGTGAAGTTCAACTGGTACGTGGACGGCGTGGAG |
| | GTGCACAACGCCAAGACCAAGCCCAGGGAGGAGCAGTACAACAGCACCTA |
| | CAGGGTGGTGAGCGTGCTGACCGTGCTGCACCAGGACTGGCTGAACGGCAA |
| | GGAGTACAAGTGCAAGGTGAGCAACAAGGCCCTGCCCGCCCCCATCGAGAA |
| | GACCATCAGCAAGGCCAAGGGCCAGCCCAGGGAGCCCCAGGTGTACACCCT |
| | GCCCCCCAGCAGGGAGGAGATGACCAAGAACCAGGTGAGCCTGACCTGCCT |
| | GGTGAAGGGCTTCTACCCCAGCGACATCGCCGTGGAGTGGGAGAGCAACGG |
| | CCAGCCCGAGAACAACTACAAGACCACCCCCCCCGTGCTGGACAGCGACGG |
| | CAGCTTCTTCCTGTACAGCAAGCTGACCGTGGACAAGAGCAGGTGGCAGCA |
| | GGGCAACGTGTTCAGCTGCAGCGTGATGCACGAGGCCCTGCACAACCACTA |
| | CACCCAGAAGAGCCTGAGCCTGAGCCCCGGCAAG |
| 56 | GAGGTGCAGCTGGTGGAGAGCGGCGGCGGCCTGGTGCAGCCCGGCGGCAG |
| | CCTGAGGCTGAGCTGCGCCGCCAGCGGCTTCACCTTCAGCAGCTACGCCATG |
| | AGCTGGGTGAGGCAGGCCCCCGGCAAGGGCCTGGAGTGGGTGAGCCAGATC |
| | AGCCCCGCCGGCGGCTACACCAACTACGCCGACAGCGTGAAGGGCAGGTTC |
| | ACCATCAGCGCCGACACCAGCAAGAACACCGCCTACCTGCAGATGAACAGC |
| | CTGAGGGCCGAGGACACCGCCGTGTACTACTGCGCCAGGGGCGAGCTGCCC |
| | TACTACCAGATGAGCAAGGTGATGGACGTGTGGGGCCAGGGCACCCTGGTG |
| | ACCGTGAGCAGCGCCAGCACCAAGGGCCCCAGCGTGTTCCCCCTGGCCCCC |
| | AGCAGCAAGAGCACCAGCGGCGGCACCGCCGCCCTGGGCTGCCTGGTGAAG |
| | GACTACTTCCCCGAGCCCGTGACCGTGAGCTGGAACAGCGGCGCCCTGACC |
| | AGCGGCGTGCACACCTTCCCCGCCGTGCTGCAGAGCAGCGGCCTGTACAGC |
| | CTGAGCAGCGTGGTGACCGTGCCCAGCAGCAGCCTGGGCACCCAGACCTAC |
| | ATCTGCAACGTGAACCACAAGCCCAGCAACACCAAGGTGGACAAGAAGGT |
| | GGAGCCCAAGAGCTGCGACAAGACCCACACCTGCCCCCCCTGCCCCGCCCC |
| | CGAGCTGCTGGGCGGCCCCAGCGTGTTCCTGTTCCCCCCCAAGCCCAAGGAC |
| | ACCCTGATGATCAGCAGGACCCCCGAGGTGACCTGCGTGGTGGTGGACGTG |
| | AGCCACGAGGACCCCGAGGTGAAGTTCAACTGGTACGTGGACGGCGTGGAG |
| | GTGCACAACGCCAAGACCAAGCCCAGGGAGGAGCAGTACAACAGCACCTA |
| | CAGGGTGGTGAGCGTGCTGACCGTGCTGCACCAGGACTGGCTGAACGGCAA |
| | GGAGTACAAGTGCAAGGTGAGCAACAAGGCCCTGCCCGCCCCCATCGAGAA |
| | GACCATCAGCAAGGCCAAGGGCCAGCCCAGGGAGCCCCAGGTGTACACCCT |
| | GCCCCCCAGCAGGGAGGAGATGACCAAGAACCAGGTGAGCCTGACCTGCCT |
| | GGTGAAGGGCTTCTACCCCAGCGACATCGCCGTGGAGTGGGAGAGCAACGG |
| | CCAGCCCGAGAACAACTACAAGACCACCCCCCCCGTGCTGGACAGCGACGG |
| | CAGCTTCTTCCTGTACAGCAAGCTGACCGTGGACAAGAGCAGGTGGCAGCA |
| | GGGCAACGTGTTCAGCTGCAGCGTGATGCACGAGGCCCTGCACAACCACTA |
| | CACCCAGAAGAGCCTGAGCCTGAGCCCCGGCAAG |
| 57 | GAGGTGCAGCTGGTGGAGAGCGGCGGCGGCCTGGTGCAGCCCGGCGGCAG |
| | CCTGAGGCTGAGCTGCGCCGCCAGCGGCTTCACCTTCAGCAGCTACGCCATG |
| | AGCTGGGTGAGGCAGGCCCCCGGCAAGGGCCTGGAGTGGGTGAGCCAGATC |
| | AGCCCCGCCGGCGGCTACACCAACTACGCCGACAGCGTGAAGGGCAGGTTC |
| | ACCATCAGCGCCGACACCAGCAAGAACACCGCCTACCTGCAGATGAACAGC |
| | CTGAGGGCCGAGGACACCGCCGTGTACTACTGCGCCAGGGGCGAGCTGCCC |
| | TTCTTCAGGATGAGCCAGGTGATGGACGTGTGGGGCCAGGGCACCCTGGTG |
| | ACCGTGAGCAGCGCCAGCACCAAGGGCCCCAGCGTGTTCCCCCTGGCCCCC |
| | AGCAGCAAGAGCACCAGCGGCGGCACCGCCGCCCTGGGCTGCCTGGTGAAG |
| | GACTACTTCCCCGAGCCCGTGACCGTGAGCTGGAACAGCGGCGCCCTGACC |
| | AGCGGCGTGCACACCTTCCCCGCCGTGCTGCAGAGCAGCGGCCTGTACAGC |
| | CTGAGCAGCGTGGTGACCGTGCCCAGCAGCAGCCTGGGCACCCAGACCTAC |
| | ATCTGCAACGTGAACCACAAGCCCAGCAACACCAAGGTGGACAAGAAGGT |
| | GGAGCCCAAGAGCTGCGACAAGACCCACACCTGCCCCCCCTGCCCCGCCCC |
| | CGAGCTGCTGGGCGGCCCCAGCGTGTTCCTGTTCCCCCCCAAGCCCAAGGAC |
| | ACCCTGATGATCAGCAGGACCCCCGAGGTGACCTGCGTGGTGGTGGACGTG |
| | AGCCACGAGGACCCCGAGGTGAAGTTCAACTGGTACGTGGACGGCGTGGAG |
| | GTGCACAACGCCAAGACCAAGCCCAGGGAGGAGCAGTACAACAGCACCTA |
| | CAGGGTGGTGAGCGTGCTGACCGTGCTGCACCAGGACTGGCTGAACGGCAA |
| | GGAGTACAAGTGCAAGGTGAGCAACAAGGCCCTGCCCGCCCCCATCGAGAA |
| | GACCATCAGCAAGGCCAAGGGCCAGCCCAGGGAGCCCCAGGTGTACACCCT |
| | GCCCCCCAGCAGGGAGGAGATGACCAAGAACCAGGTGAGCCTGACCTGCCT |
| | GGTGAAGGGCTTCTACCCCAGCGACATCGCCGTGGAGTGGGAGAGCAACGG |
| | CCAGCCCGAGAACAACTACAAGACCACCCCCCCCGTGCTGGACAGCGACGG |
| | CAGCTTCTTCCTGTACAGCAAGCTGACCGTGGACAAGAGCAGGTGGCAGCA |
| | GGGCAACGTGTTCAGCTGCAGCGTGATGCACGAGGCCCTGCACAACCACTA |
| | CACCCAGAAGAGCCTGAGCCTGAGCCCCGGCAAG |
| 58 | GAGGTGCAGCTGGTGGAGAGCGGCGGCGGCCTGGTGCAGCCCGGCGGCAG |
| | CCTGAGGCTGAGCTGCGCCGCCAGCGGCTTCACCTTCAGCAGCTACGCCATG |
| | AGCTGGGTGAGGCAGGCCCCCGGCAAGGGCCTGGAGTGGGTGAGCCAGATC |
| | AGCCCCGCCGGCGGCTACACCAACTACGCCGACAGCGTGAAGGGCAGGTTC |
| | ACCATCAGCGCCGACACCAGCAAGAACACCGCCTACCTGCAGATGAACAGC |
| | CTGAGGGCCGAGGACACCGCCGTGTACTACTGCGCCAGGGGCGAGCTGCCC |
| | TACTTCAGGATGAGCAAGGTGATGGACGTGTGGGGCCAGGGCACCCTGGTG |
| | ACCGTGAGCAGCGCCAGCACCAAGGGCCCCAGCGTGTTCCCCCTGGCCCCC |
| | AGCAGCAAGAGCACCAGCGGCGGCACCGCCGCCCTGGGCTGCCTGGTGAAG |
| | GACTACTTCCCCGAGCCCGTGACCGTGAGCTGGAACAGCGGCGCCCTGACC |
| | AGCGGCGTGCACACCTTCCCCGCCGTGCTGCAGAGCAGCGGCCTGTACAGC |
| | CTGAGCAGCGTGGTGACCGTGCCCAGCAGCAGCCTGGGCACCCAGACCTAC |
| | ATCTGCAACGTGAACCACAAGCCCAGCAACACCAAGGTGGACAAGAAGGT |
| | GGAGCCCAAGAGCTGCGACAAGACCCACACCTGCCCCCCCTGCCCCGCCCC |
| | CGAGCTGCTGGGCGGCCCCAGCGTGTTCCTGTTCCCCCCCAAGCCCAAGGAC |
| | ACCCTGATGATCAGCAGGACCCCCGAGGTGACCTGCGTGGTGGTGGACGTG |
| | AGCCACGAGGACCCCGAGGTGAAGTTCAACTGGTACGTGGACGGCGTGGAG |
| | GTGCACAACGCCAAGACCAAGCCCAGGGAGGAGCAGTACAACAGCACCTA |
| | CAGGGTGGTGAGCGTGCTGACCGTGCTGCACCAGGACTGGCTGAACGGCAA |
| | GGAGTACAAGTGCAAGGTGAGCAACAAGGCCCTGCCCGCCCCCATCGAGAA |
| | GACCATCAGCAAGGCCAAGGGCCAGCCCAGGGAGCCCCAGGTGTACACCCT |
| | GCCCCCCAGCAGGGAGGAGATGACCAAGAACCAGGTGAGCCTGACCTGCCT |
| | GGTGAAGGGCTTCTACCCCAGCGACATCGCCGTGGAGTGGGAGAGCAACGG |
| | CCAGCCCGAGAACAACTACAAGACCACCCCCCCCGTGCTGGACAGCGACGG |
| | CAGCTTCTTCCTGTACAGCAAGCTGACCGTGGACAAGAGCAGGTGGCAGCA |
| | GGGCAACGTGTTCAGCTGCAGCGTGATGCACGAGGCCCTGCACAACCACTA |
| | CACCCAGAAGAGCCTGAGCCTGAGCCCCGGCAAG |
| 59 | GAGGTGCAGCTGCTGGAGAGCGGCGGCGGCCTGGTGCAGCCCGGCGGCAGC |
| | CTGAGGCTGAGCTGCGCCGCCAGCGGCTTCACCTTCAGCAGCTACGCCATG |
| | AGCTGGGTGAGGCAGGCCCCCGGCAAGGGCCTGGAGTGGGTGAGCCAGATC |
| | AGCCCCGCCGGCGGCTACACCAACTACGCCGACAGCGTGAAGGGCAGGTTC |
| | ACCATCAGCAGGGACAACAGCAAGAACACCCTGTACCTGCAGATGAACAGC |
| | CTGAGGGCCGAGGACACCGCCGTGTACTACTGCGCCAGGGGCGAGCTGCCC |
| | TACTTCAGGATGAGCAAGGTGATGGACGTGTGGGGCCAGGGCACCCTGGTG |
| | ACCGTGAGCAGCGCCAGCACCAAGGGCCCCAGCGTGTTCCCCCTGGCCCCC |
| | AGCAGCAAGAGCACCAGCGGCGGCACCGCCGCCCTGGGCTGCCTGGTGAAG |
| | GACTACTTCCCCGAGCCCGTGACCGTGAGCTGGAACAGCGGCGCCCTGACC |
| | AGCGGCGTGCACACCTTCCCCGCCGTGCTGCAGAGCAGCGGCCTGTACAGC |
| | CTGAGCAGCGTGGTGACCGTGCCCAGCAGCAGCCTGGGCACCCAGACCTAC |
| | ATCTGCAACGTGAACCACAAGCCCAGCAACACCAAGGTGGACAAGAAGGT |
| | GGAGCCCAAGAGCTGCGACAAGACCCACACCTGCCCCCCCTGCCCCGCCCC |
| | CGAGCTGCTGGGCGGCCCCAGCGTGTTCCTGTTCCCCCCCAAGCCCAAGGAC |
| | ACCCTGATGATCAGCAGGACCCCCGAGGTGACCTGCGTGGTGGTGGACGTG |
| | AGCCACGAGGACCCCGAGGTGAAGTTCAACTGGTACGTGGACGGCGTGGAG |
| | GTGCACAACGCCAAGACCAAGCCCAGGGAGGAGCAGTACAACAGCACCTA |
| | CAGGGTGGTGAGCGTGCTGACCGTGCTGCACCAGGACTGGCTGAACGGCAA |
| | GGAGTACAAGTGCAAGGTGAGCAACAAGGCCCTGCCCGCCCCCATCGAGAA |
| | GACCATCAGCAAGGCCAAGGGCCAGCCCAGGGAGCCCCAGGTGTACACCCT |
| | GCCCCCCAGCAGGGAGGAGATGACCAAGAACCAGGTGAGCCTGACCTGCCT |
| | GGTGAAGGGCTTCTACCCCAGCGACATCGCCGTGGAGTGGGAGAGCAACGG |
| | CCAGCCCGAGAACAACTACAAGACCACCCCCCCCGTGCTGGACAGCGACGG |
| | CAGCTTCTTCCTGTACAGCAAGCTGACCGTGGACAAGAGCAGGTGGCAGCA |
| | GGGCAACGTGTTCAGCTGCAGCGTGATGCACGAGGCCCTGCACAACCACTA |
| | CACCCAGAAGAGCCTGAGCCTGAGCCCCGGCAAG |
| 60 | GAGGTGCAGCTGCTGGAGAGCGGCGGCGGCCTGGTGCAGCCCGGCGGCAGC |
| | CTGAGGCTGAGCTGCGCCGCCAGCGGCTTCACCTTCAGCAGCTACGCCATG |
| | AGCTGGGTGAGGCAGGCCCCCGGCAAGGGCCTGGAGTGGGTGAGCCAGATC |
| | AGCCCCGCCGGCGGCTACACCAACTACGCCGACAGCGTGAAGGGCAGGTTC |
| | ACCATCAGCAGGGACAACAGCAAGAACACCCTGTACCTGCAGATGAACAGC |
| | CTGAGGGCCGAGGACACCGCCGTGTACTACTGCGCCAGGGGCGAGCTGCCC |
| | TACTACCAGATGAGCAAGGTGATGGACGTGTGGGGCCAGGGCACCCTGGTG |
| | ACCGTGAGCAGCGCCAGCACCAAGGGCCCCAGCGTGTTCCCCCTGGCCCCC |
| | AGCAGCAAGAGCACCAGCGGCGGCACCGCCGCCCTGGGCTGCCTGGTGAAG |
| | GACTACTTCCCCGAGCCCGTGACCGTGAGCTGGAACAGCGGCGCCCTGACC |
| | AGCGGCGTGCACACCTTCCCCGCCGTGCTGCAGAGCAGCGGCCTGTACAGC |
| | CTGAGCAGCGTGGTGACCGTGCCCAGCAGCAGCCTGGGCACCCAGACCTAC |
| | ATCTGCAACGTGAACCACAAGCCCAGCAACACCAAGGTGGACAAGAAGGT |
| | GGAGCCCAAGAGCTGCGACAAGACCCACACCTGCCCCCCCTGCCCCGCCCC |
| | CGAGCTGCTGGGCGGCCCCAGCGTGTTCCTGTTCCCCCCCAAGCCCAAGGAC |
| | ACCCTGATGATCAGCAGGACCCCCGAGGTGACCTGCGTGGTGGTGGACGTG |
| | AGCCACGAGGACCCCGAGGTGAAGTTCAACTGGTACGTGGACGGCGTGGAG |
| | GTGCACAACGCCAAGACCAAGCCCAGGGAGGAGCAGTACAACAGCACCTA |
| | CAGGGTGGTGAGCGTGCTGACCGTGCTGCACCAGGACTGGCTGAACGGCAA |
| | GGAGTACAAGTGCAAGGTGAGCAACAAGGCCCTGCCCGCCCCCATCGAGAA |
| | GACCATCAGCAAGGCCAAGGGCCAGCCCAGGGAGCCCCAGGTGTACACCCT |
| | GCCCCCCAGCAGGGAGGAGATGACCAAGAACCAGGTGAGCCTGACCTGCCT |
| | GGTGAAGGGCTTCTACCCCAGCGACATCGCCGTGGAGTGGGAGAGCAACGG |
| | CCAGCCCGAGAACAACTACAAGACCACCCCCCCCGTGCTGGACAGCGACGG |
| | CAGCTTCTTCCTGTACAGCAAGCTGACCGTGGACAAGAGCAGGTGGCAGCA |
| | GGGCAACGTGTTCAGCTGCAGCGTGATGCACGAGGCCCTGCACAACCACTA |
| | CACCCAGAAGAGCCTGAGCCTGAGCCCCGGCAAG |
| 61 | GAGGTGCAGCTGCTGGAGAGCGGCGGCGGCCTGGTGCAGCCCGGCGGCAGC |
| | CTGAGGCTGAGCTGCGCCGCCAGCGGCTTCACCTTCAGCAGCTACGCCATG |
| | AGCTGGGTGAGGCAGGCCCCCGGCAAGGGCCTGGAGTGGGTGAGCCAGATC |
| | AGCCCCGCCGGCGGCTACACCAACTACGCCGACAGCGTGAAGGGCAGGTTC |
| | ACCATCAGCAGGGACAACAGCAAGAACACCCTGTACCTGCAGATGAACAGC |
| | CTGAGGGCCGAGGACACCGCCGTGTACTACTGCGCCAGGGGCGAGCTGCCC |
| | TTCTTCAGGATGAGCCAGGTGATGGACGTGTGGGGCCAGGGCACCCTGGTG |
| | ACCGTGAGCAGCGCCAGCACCAAGGGCCCCAGCGTGTTCCCCCTGGCCCCC |
| | AGCAGCAAGAGCACCAGCGGCGGCACCGCCGCCCTGGGCTGCCTGGTGAAG |
| | GACTACTTCCCCGAGCCCGTGACCGTGAGCTGGAACAGCGGCGCCCTGACC |
| | AGCGGCGTGCACACCTTCCCCGCCGTGCTGCAGAGCAGCGGCCTGTACAGC |
| | CTGAGCAGCGTGGTGACCGTGCCCAGCAGCAGCCTGGGCACCCAGACCTAC |
| | ATCTGCAACGTGAACCACAAGCCCAGCAACACCAAGGTGGACAAGAAGGT |
| | GGAGCCCAAGAGCTGCGACAAGACCCACACCTGCCCCCCCTGCCCCGCCCC |
| | CGAGCTGCTGGGCGGCCCCAGCGTGTTCCTGTTCCCCCCCAAGCCCAAGGAC |
| | ACCCTGATGATCAGCAGGACCCCCGAGGTGACCTGCGTGGTGGTGGACGTG |
| | AGCCACGAGGACCCCGAGGTGAAGTTCAACTGGTACGTGGACGGCGTGGAG |
| | GTGCACAACGCCAAGACCAAGCCCAGGGAGGAGCAGTACAACAGCACCTA |
| | CAGGGTGGTGAGCGTGCTGACCGTGCTGCACCAGGACTGGCTGAACGGCAA |
| | GGAGTACAAGTGCAAGGTGAGCAACAAGGCCCTGCCCGCCCCCATCGAGAA |
| | GACCATCAGCAAGGCCAAGGGCCAGCCCAGGGAGCCCCAGGTGTACACCCT |
| | GCCCCCCAGCAGGGAGGAGATGACCAAGAACCAGGTGAGCCTGACCTGCCT |
| | GGTGAAGGGCTTCTACCCCAGCGACATCGCCGTGGAGTGGGAGAGCAACGG |
| | CCAGCCCGAGAACAACTACAAGACCACCCCCCCCGTGCTGGACAGCGACGG |
| | CAGCTTCTTCCTGTACAGCAAGCTGACCGTGGACAAGAGCAGGTGGCAGCA |
| | GGGCAACGTGTTCAGCTGCAGCGTGATGCACGAGGCCCTGCACAACCACTA |
| | CACCCAGAAGAGCCTGAGCCTGAGCCCCGGCAAG |
| 62 | GACATCCAGATGACCCAGAGCCCCAGCAGCCTGAGCGCCAGCGTGGGCGAC |
| | AGGGTGACCATCACCTGCAGGGCCAGCCAGTTCCTGAGCAGCTACCTGGCC |
| | TGGTACCAGCAGAAGCCCGGCAAGGCCCCCAAGCTGCTGATCTACGGCGCC |
| | AGCGCCAGGGCCAGCGGCGTGCCCAGCAGGTTCAGCGGCAGCGGCAGCGG |
| | CACCGACTTCACCCTGACCATCAGCAGCCTGCAGCCCGAGGACTTCGCCACC |
| | TACTACTGCCAGCAGTACCTGGCCAGCCCCGCCACCTTCGGCCAGGGCACC |
| | AAGGTGGAGATCAAGAGGACCGTGGCCGCCCCCAGCGTGTTCATCTTCCCC |
| | CCCAGCGACGAGCAGCTGAAGAGCGGCACCGCCAGCGTGGTGTGCCTGCTG |
| | AACAACTTCTACCCCAGGGAGGCCAAGGTGCAGTGGAAGGTGGACAACGCC |
| | CTGCAGAGCGGCAACAGCCAGGAGAGCGTGACCGAGCAGGACAGCAAGGA |
| | CAGCACCTACAGCCTGAGCAGCACCCTGACCCTGAGCAAGGCCGACTACGA |
| | GAAGCACAAGGTGTACGCCTGCGAGGTGACCCACCAGGGCCTGAGCAGCCC |
| | CGTGACCAAGAGCTTCAACAGGGGCGAGTGC |
| 63 | GACATCCAGATGACCCAGAGCCCCAGCAGCCTGAGCGCCAGCGTGGGCGAC |
| | AGGGTGACCATCACCTGCAGGGCCAGCCAGTACTTCAGCAGCTACCTGGCC |
| | TGGTACCAGCAGAAGCCCGGCAAGGCCCCCAAGCTGCTGATCTACGGCGCC |
| | AGCGCCAGGGCCAGCGGCGTGCCCAGCAGGTTCAGCGGCAGCGGCAGCGG |
| | CACCGACTTCACCCTGACCATCAGCAGCCTGCAGCCCGAGGACTTCGCCACC |
| | TACTACTGCCAGCAGTACCTGGCCAGCCCCGCCACCTTCGGCCAGGGCACC |
| | AAGGTGGAGATCAAGAGGACCGTGGCCGCCCCCAGCGTGTTCATCTTCCCC |
| | CCCAGCGACGAGCAGCTGAAGAGCGGCACCGCCAGCGTGGTGTGCCTGCTG |
| | AACAACTTCTACCCCAGGGAGGCCAAGGTGCAGTGGAAGGTGGACAACGCC |
| | CTGCAGAGCGGCAACAGCCAGGAGAGCGTGACCGAGCAGGACAGCAAGGA |
| | CAGCACCTACAGCCTGAGCAGCACCCTGACCCTGAGCAAGGCCGACTACGA |
| | GAAGCACAAGGTGTACGCCTGCGAGGTGACCCACCAGGGCCTGAGCAGCCC |
| | CGTGACCAAGAGCTTCAACAGGGGCGAGTGC |
| 64 | GACATCCAGATGACCCAGAGCCCCAGCAGCCTGAGCGCCAGCGTGGGCGAC |
| | AGGGTGACCATCACCTGCAGGGCCAGCCAGTTCCTGAGCAGCTACCTGGCC |
| | TGGTACCAGCAGAAGCCCGGCAAGGCCCCCAAGCTGCTGATCTACGGCGCC |
| | AGCGCCAGGGCCAGCGGCGTGCCCAGCAGGTTCAGCGGCAGCGGCAGCGG |
| | CACCGACTTCACCCTGACCATCAGCAGCCTGCAGCCCGAGGACTTCGCCACC |
| | TACTACTGCCAGCAGTACCTGGCCAGCCCCGCCACCTTCGGCCAGGGCACC |
| | AAGGTGGAGATCAAGAGGACCGTGGCCGCCCCCAGCGTGTTCATCTTCCCC |
| | CCCAGCGACGAGCAGCTGAAGAGCGGCACCGCCAGCGTGGTGTGCCTGCTG |
| | AACAACTTCTACCCCAGGGAGGCCAAGGTGCAGTGGAAGGTGGACAACGCC |
| | CTGCAGAGCGGCAACAGCCAGGAGAGCGTGACCGAGCAGGACAGCAAGGA |
| | CAGCACCTACAGCCTGAGCAGCACCCTGACCCTGAGCAAGGCCGACTACGA |
| | GAAGCACAAGGTGTACGCCTGCGAGGTGACCCACCAGGGCCTGAGCAGCCC |
| | CGTGACCAAGAGCTTCAACAGGGGCGAGTGC |
| 65 | GACATCCAGATGACCCAGAGCCCCAGCAGCCTGAGCGCCAGCGTGGGCGAC |
| | AGGGTGACCATCACCTGCAGGGCCAGCCAGTTCCTGAGCAGCTTCCTGGCCT |
| | GGTACCAGCAGAAGCCCGGCAAGGCCCCCAAGCTGCTGATCTACGGCGCCA |
| | GCGCCAGGGCCAGCGGCGTGCCCAGCAGGTTCAGCGGCAGCGGCAGCGGC |
| | ACCGACTTCACCCTGACCATCAGCAGCCTGCAGCCCGAGGACTTCGCCACCT |
| | ACTACTGCCAGCAGTACCTGGCCAGCCCCGCCACCTTCGGCCAGGGCACCA |
| | AGGTGGAGATCAAGAGGACCGTGGCCGCCCCCAGCGTGTTCATCTTCCCCC |
| | CCAGCGACGAGCAGCTGAAGAGCGGCACCGCCAGCGTGGTGTGCCTGCTGA |
| | ACAACTTCTACCCCAGGGAGGCCAAGGTGCAGTGGAAGGTGGACAACGCCC |
| | TGCAGAGCGGCAACAGCCAGGAGAGCGTGACCGAGCAGGACAGCAAGGAC |
| | AGCACCTACAGCCTGAGCAGCACCCTGACCCTGAGCAAGGCCGACTACGAG |
| | AAGCACAAGGTGTACGCCTGCGAGGTGACCCACCAGGGCCTGAGCAGCCCC |
| | GTGACCAAGAGCTTCAACAGGGGCGAGTGC |
| 66 | GAGATCGTGCTGACCCAGAGCCCCGGCACCCTGAGCCTGAGCCCCGGCGAG |
| | AGGGCCACCCTGAGCTGCAGGGCCAGCCAGTTCCTGAGCAGCTACCTGGCC |
| | TGGTACCAGCAGAAGCCCGGCCAGGCCCCCAGGCTGCTGATCTACGGCGCC |
| | AGCGCCAGGGCCAGCGGCATCCCCGACAGGTTCAGCGGCAGCGGCAGCGGC |
| | ACCGACTTCACCCTGACCATCAGCAGGCTGGAGCCCGAGGACTTCGCCGTG |
| | TACTACTGCCAGCAGTACCTGGCCAGCCCCGCCACCTTCGGCCAGGGCACC |
| | AAGGTGGAGATCAAGAGGACCGTGGCCGCCCCCAGCGTGTTCATCTTCCCC |
| | CCCAGCGACGAGCAGCTGAAGAGCGGCACCGCCAGCGTGGTGTGCCTGCTG |
| | AACAACTTCTACCCCAGGGAGGCCAAGGTGCAGTGGAAGGTGGACAACGCC |
| | CTGCAGAGCGGCAACAGCCAGGAGAGCGTGACCGAGCAGGACAGCAAGGA |
| | CAGCACCTACAGCCTGAGCAGCACCCTGACCCTGAGCAAGGCCGACTACGA |
| | GAAGCACAAGGTGTACGCCTGCGAGGTGACCCACCAGGGCCTGAGCAGCCC |
| | CGTGACCAAGAGCTTCAACAGGGGCGAGTGC |
| 67 | GAGATCGTGCTGACCCAGAGCCCCGGCACCCTGAGCCTGAGCCCCGGCGAG |
| | AGGGCCACCCTGAGCTGCAGGGCCAGCCAGTTCCTGAGCAGCTACCTGGCC |
| | TGGTACCAGCAGAAGCCCGGCCAGGCCCCCAGGCTGCTGATCTACGGCGCC |
| | AGCGCCAGGGCCAGCGGCATCCCCGACAGGTTCAGCGGCAGCGGCAGCGGC |
| | ACCGACTTCACCCTGACCATCAGCAGGCTGGAGCCCGAGGACTTCGCCGTG |
| | TACTACTGCCAGCAGTACCTGGCCAGCCCCGCCACCTTCGGCCAGGGCACC |
| | AAGGTGGAGATCAAGAGGACCGTGGCCGCCCCCAGCGTGTTCATCTTCCCC |
| | CCCAGCGACGAGCAGCTGAAGAGCGGCACCGCCAGCGTGGTGTGCCTGCTG |
| | AACAACTTCTACCCCAGGGAGGCCAAGGTGCAGTGGAAGGTGGACAACGCC |
| | CTGCAGAGCGGCAACAGCCAGGAGAGCGTGACCGAGCAGGACAGCAAGGA |
| | CAGCACCTACAGCCTGAGCAGCACCCTGACCCTGAGCAAGGCCGACTACGA |
| | GAAGCACAAGGTGTACGCCTGCGAGGTGACCCACCAGGGCCTGAGCAGCCC |
| | CGTGACCAAGAGCTTCAACAGGGGCGAGTGC |
| 68 | GAGATCGTGCTGACCCAGAGCCCCGGCACCCTGAGCCTGAGCCCCGGCGAG |
| | AGGGCCACCCTGAGCTGCAGGGCCAGCCAGTTCCTGAGCAGCTACCTGGCC |
| | TGGTACCAGCAGAAGCCCGGCCAGGCCCCCAGGCTGCTGATCTACGGCGCC |
| | AGCGCCAGGGCCAGCGGCATCCCCGACAGGTTCAGCGGCAGCGGCAGCGGC |
| | ACCGACTTCACCCTGACCATCAGCAGGCTGGAGCCCGAGGACTTCGCCGTG |
| | TACTACTGCCAGCAGTACCTGGCCAGCCCCGCCACCTTCGGCCAGGGCACC |
| | AAGGTGGAGATCAAGAGGACCGTGGCCGCCCCCAGCGTGTTCATCTTCCCC |
| | CCCAGCGACGAGCAGCTGAAGAGCGGCACCGCCAGCGTGGTGTGCCTGCTG |
| | AACAACTTCTACCCCAGGGAGGCCAAGGTGCAGTGGAAGGTGGACAACGCC |
| | CTGCAGAGCGGCAACAGCCAGGAGAGCGTGACCGAGCAGGACAGCAAGGA |
| | CAGCACCTACAGCCTGAGCAGCACCCTGACCCTGAGCAAGGCCGACTACGA |
| | GAAGCACAAGGTGTACGCCTGCGAGGTGACCCACCAGGGCCTGAGCAGCCC |
| | CGTGACCAAGAGCTTCAACAGGGGCGAGTGC |
| 69 | QVQLQESGPGLVKPSETLSLTCTVSGGSVSSGDYYWTWIRQSPGKGLEWIGHIYYSGN |
| | TNYNPSLKSRLTISIDTSKTQFSLKLSSVTAADTAIYYCVRDRVTGAFDIWGQGTMVTVSS |
| | (Anti-EGFR VH AA Seq) |
| 70 | DIQMTQSPSSLSASVGDRVTITCQASQDISNYLNWYQQKPGKAPKLLIYDASNLETGVP |
| | SRFSGSGSGTDFTFTISSLQPEDIATYFCQHFDHLPLAFGGGTKVEIK |
| | (Anti-EGFR VL AA Seq) |
| 71 | SGDYYWT |
| | (Anti-EGFR VH CDR1 AA Seq) |
| 72 | HIYYSGNTNYNPSLK |
| | (Anti-EGFR VH CDR2 AA Seq) |
| 73 | RVTGAFDI |
| | (Anti-EGFR VH CDR3 AA Seq) |
| 74 | QASQDISNYL |
| | (Anti-EGFR VL CDR1 AA Seq) |
| 75 | ASNLET |
| | (Anti-EGFR VL CDR2 AA Seq) |
| 76 | QHFDHLPLA |
| | (Anti-EGFR VL CDR3 AA Seq) |
| 77 | EVQLLESGGGLVQPGGSLRLSCAASGFTFSSYAMSWVRQAPGKGLEWVSQISPAGGYTN |
| | YADSVKGRFTISRDNSKNTLYLQMNSLRAEDTAVYYCARGELPYFRMSKVMDVWGQGTL |
| | VTVSS |
| | (Anti-NRP1 VH AA Seq) |
| 78 | EIVLTQSPGTLSLSPGERATLSCRASQFLSSYLAWYQQKPGQAPRLLIYGASARASG |
| | IPDRFSGSdGSGTDFTLTISRLEPEDFAVYYCQQYLASPATFGQGTKVEIK |
| | (Anti-NRP1 VL AA Seq) |
| 79 | FTFSSYAM |
| | (Anti-NRP1 VH CDR1 AA Seq) |
| 80 | QISPAGGYTNYADSVK |
| | (Anti-NRP1 VH CDR2 AA Seq) |
| 81 | GELPYFRMSKVMDV |
| | (Anti-NRP1 VH CDR3 AA Seq) |
| 82 | GELPYHRMSKVMDV |
| | (Anti-NRP1 VH CDR3 AA Seq) |
| 83 | GELPYYQMSKVMDV |
| | (Anti-NRP1 VH CDR3 AA Seq) |
| 84 | GELPFFRMSQVMDV |
| | (Anti-NRP1 VH CDR3 AA Seq) |
| 85 | RASQFLSSYLA |
| | (Anti-NRP1 VL CDR1 AA Seq) |
| 86 | RASQYFSSYLA |
| | (Anti-NRP1 VL CDR1 AA Seq) |
| 87 | RASQFLSSFLA |
| | (Anti-NRP1 VL CDR1 AA Seq) |
| 88 | GASARAS |
| | (Anti-NRP1 VL CDR2 AA Seq) |
| 89 | QQYLASPAT |
| | (Anti-NRP1 VL CDR3 AA Seq) |

1. A method of enhancing effects of a therapeutic agent in a subject having a cancer that is resistant or refractory to the therapeutic agent, the method comprising:
administering to the subject (i) the therapeutic agent; and (ii) a target protein degrader, such that the effects of the therapeutic agent are enhanced as compared to administering the therapeutic agent alone;
wherein the target protein degrader comprises a bispecific binding molecule comprising:
(a) a target protein binding domain that specifically binds to a target protein on the subject's cancer cells; and
(b) a neuropilin-1 (NRP1) binding domain that binds to NRP1 comprising an antibody or NRP-1-binding fragment thereof.

2. The method of claim 1, wherein the target protein is a receptor tyrosine kinase (RTK).

3. The method of claim 2, wherein the receptor tyrosine kinase is selected from epidermal growth factor receptors (EGFRs), platelet-derived growth factor receptors (PDGFRs), fibroblast growth factor receptors (FGFRs), Met receptors tyrosine kinase (METs), and vascular endothelial growth factors (VEGFRs).

4. The method of claim 2, wherein the receptor tyrosine kinase is EGFR.

5. The method of claim 2, wherein the receptor tyrosine kinase is cMET.

6. The method of claim 2, wherein the receptor tyrosine kinase is HER2.

7. The method of claim 2, wherein the receptor tyrosine kinase is IGF1R.

8. The method of claim 1, wherein the target cell is a cancer cell.

9. The method of claim 8, wherein the cancer cell is selected from the group consisting of lung cancer, breast cancer, colon and rectum cancer, head and neck cancer, esophagogastric cancer, liver cancer, glioblastoma, prostate cancer, cervical cancer, ovarian cancer, bladder cancer, kidney cancer, and pancreatic cancer.

10. The method of claim 8, wherein the cancer cell is a non-small cell lung cancer (NSCLC) cell.

11. The method of claim 1, wherein the target protein binding domain and the NRP1 binding domain are each independently selected from the group consisting of IgG, half antibodies, single-domain antibodies, nanobodies, Fabs, monospecific Fab2, Fc, scFv, minibodies, IgNAR, V-NAR, hcIgG, VHH domain, camelid antibodies, and peptibodies.

12. The method of claim 1, wherein the NRP1 binding domain comprises:
(i) an antibody heavy chain variable (VH) domain comprising CDR1, CDR2 and CDR3 regions (HCDR1, HCDR2 and HCDR3, respectively), wherein HCDR1 consists of the sequence shown in SEQ ID NO: 79, HCDR2 consists of the sequence shown in SEQ ID NO: 80, and HCDR3 consists of the sequence shown in any one of SEQ ID NOs: 81-84; and
(ii) an antibody light chain variable (VL) domain comprising CDR1, CDR2 and CDR3 regions (LCDR1, LCDR2 and LCDR3, respectively), wherein LCDR1 consists of the sequence shown in any one of SEQ ID NOs: 85-87, LCDR2 consists of the sequence shown in SEQ ID NO: 88, and LCDR3 consists of the sequence shown in SEQ ID NO: 89.

13. The method of claim 12, wherein:
(iii) HCDR1 consists of the sequence shown in SEQ ID NO: 79, HCDR2 consists of the sequence shown in SEQ ID NO: 80, and HCDR3 consists of the sequence shown in any one of SEQ ID NO: 84; and
(iv) LCDR1 consists of the sequence shown in SEQ ID NO: 85, LCDR2 consists of the sequence shown in SEQ ID NO: 88, and LCDR3 consists of the sequence shown in SEQ ID NO: 89.

14. The method of claim 1, wherein the enhanced effects comprise increased tumor growth inhibition.

15. The method of claim 1, wherein the enhanced effects comprise increased median survival time.

16. The method of claim 1, wherein the therapeutic agent targets the EGFR pathway.

17. The method of claim 16, wherein the therapeutic agent is selected from the group consisting of Lazertinib, Osimertinib (AZD9291), WZ4002, Cyasterone, Erlotinib (OSI-774) HCl, efitinib (ZD1839), Lapatinib (GW-572016) Ditosylate, Afatinib (BIBW2992), Saracatinib (AZD0530), Vandetanib (ZD6474), Neratinib (HKI-272), Canertinib (CI-1033), Lapatinib (GW-572016), AG-490 (Tyrphostin B42), CP-724714, Dacomitinib (PF-00299804), Sapitinib (AZD8931), CUDC-101, AG-1478 (Tyrphostin AG-1478), PD153035 HCl, Pelitinib (EKB-569), AC480 (BMS-599626), AEE788 (NVP-AEE788), AP26113-analog (ALK-IN-1), OSI-420, WZ3146, HER2-Inhibitor-1, WZ8040, Allitinib tosylate, Rociletinib (CO-1686), Genistein (NPI 031L), Varlitinib, TQB33804 (EGFR-IN-7), Icotinib (BPI-2009H), TAK-285, Daphnetin, Tyrphostin 9, AG-18, AG 555, AZ5104, CL-387785 (EKI-785), Tyrphostin AG-258, AG-556, Tucatinib, Erlotinib (OSI-774), Gefitinib-based PROTAC 3, Zorifertinib (AZD 3759), ErbB2, AV-412 free base, AST-1306, JND3229, BI-4020, Theliatinib (HMPL-309), BDTX-189, Lifirafenib (BGB-283), Pyrotinib (SHR-1258), O-Demethyl-Gefitinib, Epertinib hydrochloride, SU5214, Avitinib (ACO0010), AG 494, and Poziotinib (HM781-36B).

18. The method of claim 16, wherein the therapeutic agent is Osimertinib.

19. The method of claim 1, wherein the therapeutic agent targets the cMET pathway.

20. The method of claim 19, wherein the therapeutic agent is selected from the group consisting of Crizotinib, Cabozantinib, Foretinib, PHA-665752, SU11274, SGX-523, BMS-777607, Tivantinib, JNJ-38877605, PF-04217903, Amuvatinib (MP-470), MGCD-265 analog, Capmatinib, BMS-754807, BMS-794833, AMG-208, MK-2461, Golvatinib, AMG-458, NVP-BVU972, AMG 337, Merestinib, JNJ-38877618, Crizotinib hydrochlorode, Ningetinib, AMG-1, UNC2025, Pamufetinib, Altiratinib, NPS-1304, Savolitinib.

21. The method of claim 19, wherein the therapeutic agent is Crizotinib.

22. The method of claim 1, wherein the therapeutic agent targets a KRAS protein.

23. The method of claim 22, wherein the therapeutic agent is selected from the group consisting of MRTX1133, Sotorasib (AMG510), Adagrasib (MRTX849), LC-2, Deltarasin, BAY-293, ARS-1620, BI-2852, ARS-853 (ARS853), BI-3406, ASP2453, Sotorasib (AMG510) racemate, Pan-RAS-IN-1, MRTX-1257, Zoledronic acid (ZOL 446), Lonafarnib (SCH66336), K-Ras (G12C) inhibitor 9, Salirasib, Alamandine, (Rac)-Antineoplaston A10, K-Ras-IN-1, MCP110, 6H05, K-Ras (G12C) inhibitor 12, Kobe0065, K-Ras (G12C) inhibitor 6, BQU57, Kobe2602, NAV-2729, Antineoplaston A10, Fendiline hydrochloride, KRpep-2d, Pei-illy′ alcohol, RBC8, KY1220, CID-1067700, and Zoledronic acid monohydrate.

24. The method of claim 23, wherein the therapeutic agent is sotorasib.

25. The method of claim 1, wherein the therapeutic agent targets a protein selected from the group consisting of HER2, IGF1R, ALK, Braf, VEGF, and PDGF.

26. The method of claim 1, which further comprises administering to the subject a second therapeutic agent, wherein the effects of the second therapeutic agent are enhanced by the protein target degrader as compared to administering the second therapeutic agent alone.

27. The method of claim 26, wherein the therapeutic agent targets EGFR and the second therapeutic agent targets cMET.

28. The method of claim 27, wherein the therapeutic agent is Osimertinib and the second therapeutic agent is sotorasib.

29. A method of enhancing effects of a receptor tyrosine kinase (RTK) inhibitor in a subject having a cancer that is resistant or refractory to the RTK inhibitor, the method comprising:
administering to the subject (i) the RTK inhibitor; and (ii) a target protein degrader, such that the effects of the RTK inhibitor are enhanced as compared to administering the RTK inhibitor alone;
wherein the target protein degrader comprises a bispecific binding molecule comprising:
(a) a target protein binding domain that specifically binds to an RTK on the subject's cancer cells; and
(b) a neuropilin-1 (NRP1) binding domain that binds to NRP1 comprising an antibody or NRP-1-binding fragment thereof.

30. A method of enhancing effects of an epidermal growth factor receptor (EGFR) inhibitor in a subject having a cancer that is resistant or refractory to the EGFR inhibitor, the method comprising:
administering to the subject (i) the EGFR inhibitor; and (ii) a target protein degrader, such that the effects of the EGFR inhibitor are enhanced as compared to administering the EGFR inhibitor alone;
wherein the target protein degrader comprises a bispecific binding molecule comprising:
(a) a target protein binding domain that specifically binds to EGFR on the subject's cancer cells; and
(b) a neuropilin-1 (NRP1) binding domain that binds to NRP1 comprising an antibody or NRP-1-binding fragment thereof.


SEQUENCE LISTING (89 sequences)

SEQ ID NO: 1 - protein, 474 aa, synthetic construct
  REGION 1..474: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSHTPGNSKPTRTPRR

SEQ ID NO: 2 - protein, 705 aa, synthetic construct
  REGION 1..705: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPYYRMSKVMDVWGQGTLVTVSSGGGGSGGGGSGGGGSDIQMTQSPSSLSASVGDRVTITCRASQYFSSYLAWYQQKPGKAPKLLIYGASSRASGVPSRFSGSGSGTDFTLTISSLQPEDFATYYCQQYLGSPPTFGQGTKVEIK

SEQ ID NO: 3 - protein, 705 aa, synthetic construct
  REGION 1..705: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPYFRMSKVMDVWGQGTLVTVSSGGGGSGGGGSGGGGSDIQMTQSPSSLSASVGDRVTITCRASQFLSSYLAWYQQKPGKAPKLLIYGASARASGVPSRFSGSGSGTDFTLTISSLQPEDFATYYCQQYLASPATFGQGTKVEIK

SEQ ID NO: 4 - protein, 705 aa, synthetic construct
  REGION 1..705: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPYYQMSKVMDVWGQGTLVTVSSGGGGSGGGGSGGGGSDIQMTQSPSSLSASVGDRVTITCRASQYFSSYLAWYQQKPGKAPKLLIYGASARASGVPSRFSGSGSGTDFTLTISSLQPEDFATYYCQQYLASPATFGQGTKVEIK

SEQ ID NO: 5 - protein, 705 aa, synthetic construct
  REGION 1..705: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPFFRMSQVMDVWGQGTLVTVSSGGGGSGGGGSGGGGSDIQMTQSPSSLSASVGDRVTITCRASQFLSSYLAWYQQKPGKAPKLLIYGASARASGVPSRFSGSGSGTDFTLTISSLQPEDFATYYCQQYLASPATFGQGTKVEIK

SEQ ID NO: 6 - protein, 705 aa, synthetic construct
  REGION 1..705: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPYFRMSKVMDVWGQGTLVTVSSGGGGSGGGGSGGGGSDIQMTQSPSSLSASVGDRVTITCRASQFLSSFLAWYQQKPGKAPKLLIYGASARASGVPSRFSGSGSGTDFTLTISSLQPEDFATYYCQQYLASPATFGQGTKVEIK

SEQ ID NO: 7 - protein, 709 aa, synthetic construct
  REGION 1..709: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPYFRMSKVMDVWGQGTLVTVSSGGGGSGGGGSGGGGSDIQMTQSPSSLSASVGDRVTITCRASQFLSSYLAWYQQKPGKAPKLLIYGASARASGVPSRFSGSGSGTDFTLTISSLQPEDFATYYCQQYLASPATFGQGTKVEIK

SEQ ID NO: 8 - protein, 705 aa, synthetic construct
  REGION 1..705: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPYFRMSKVMDVWGQGTLVTVSSGDGSSGGGGASDIQMTQSPSSLSASVGDRVTITCRASQFLSSYLAWYQQKPGKAPKLLIYGASARASGVPSRFSGSGSGTDFTLTISSLQPEDFATYYCQQYLASPATFGQGTKVEIK

SEQ ID NO: 9 - protein, 714 aa, synthetic construct
  REGION 1..714: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPYFRMSKVMDVWGQGTLVTVSSGGGGSGGGGSGGGGSGGGGSDIQMTQSPSSLSASVGDRVTITCRASQFLSSYLAWYQQKPGKAPKLLIYGASARASGVPSRFSGSGSGTDFTLTISSLQPEDFATYYCQQYLASPATFGQGTKVEIK

SEQ ID NO: 10 - protein, 709 aa, synthetic construct
  REGION 1..709: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQMTQSPSSLSASVGDRVTITCRASQFLSSYLAWYQQKPGKAPKLLIYGASARASGVPSRFSGSGSGTDFTLTISSLQPEDFATYYCQQYLASPATFGQGTKVEIKGGGGSGGGGSGGGGSEVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPYFRMSKVMDVWGQGTLVTVSS

SEQ ID NO: 11 - protein, 709 aa, synthetic construct
  REGION 1..709: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTFSSYAMSWVRQAPGKGLEWVSQISPAGGYTNYADSVKGRFTISRDNSKNTLYLQMNSLRAEDTAVYYCARGELPYFRMSKVMDVWGQGTLVTVSSGGGGSGGGGSGGGGSEIVLTQSPGTLSLSPGERATLSCRASQFLSSYLAWYQQKPGQAPRLLIYGASARASGIPDRFSGSGSGTDFTLTISRLEPEDFAVYYCQQYLASPATFGQGTKVEIK

SEQ ID NO: 12 - protein, 214 aa, synthetic construct
  REGION 1..214: note = Synthetic
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 5 aa, synthetic construct
  REGION 1..5: note = Synthetic
GGGGS

SEQ ID NO: 14 - DNA, 1422 bp, synthetic construct
  misc_feature 1..1422: note = Synthetic
  source 1..1422: mol_type = other DNA
caagtgcagctgcaggaatccggccccgggcttgtgaagcctagcgaaacactctcgctcacctgtactgtcagcggtggatcagtgtcctccggcgattactactggacctggattcggcagagccctggaaagggactggagtggatcggacacatctactactccgggaacactaactacaacccgtcgttgaagtccagactgacgatcagtatcgatacctccaagacccagttcagcctgaagctgagctcagtgacagccgccgacactgcaatctactactgcgtgcgggacagagtgaccggagccttcgacatctggggccagggaaccatggtcactgtgtcgtctgcgtcaaccaagggtccgtccgtgtttcccctggccccgtgctcgcggagcacctccgagtccactgccgccttgggctgcctggtcaaagactacttccctgaacccgtgactgtcagctggaactccggagctctgacctcgggagtgcacaccttcccggccgtgctgcaatcgagcggcctctactccctgtcctccgtcgtgaccgtgccatcatcaaacttcggaacccaaacttatacgtgcaacgtcgaccacaagccctccaataccaaagtcgacaagaccgtggagaggaaatgctgcgtggagtgtccgccttgccccgcgccgccggtggccggacctagcgtgttcctgttcccgccgaagccaaaggacactctcatgatctcccgcacccctgaagtcacttgcgtcgtggtggacgtttcccacgaggatcccgaagtgcagttcaattggtacgtggacggggtggaagtacataacgccaagaccaagcccagggaagaacagtttaactccaccttccgggtggtgtcggtgctcactgtggtgcatcaggattggctcaatggaaaggagtacaagtgcaaagtgtcgaacaagggtctgcccgctcctattgaaaagaccatttccaaaaccaagggacagcccagagagcctcaggtctacaccctgcctccgagccgcgaggaaatgaccaagaaccaagtgtctctgacttgcctcgtgaagggattctacccctccgatatcgcggtggagtgggagagcaacgggcagccagagaacaactataagaccaccccgcctatgctggactccgatggctccttcttcttgtactcgaagctgaccgtggacaagtcccgctggcaacagggaaacgtgttcagctgtagcgtgatgcacgaagccctgcacaaccactacacccagaagtccctgtcgctttcccccgggaagggcggtggcggatccggcggcgggggcagcgggggcggcggttcccataccccggggaactcaaagcccacccggactccacggcgc

SEQ ID NO: 15 - DNA, 2121 bp, synthetic construct
  misc_feature 1..2121: note = Synthetic
  source 1..2121: mol_type = other DNA
caggttcagctgcaagagtctggccctggcctggtcaagccttccgaaacactgtctctgacctgcaccgtgtctggcggctctgtgtcctctggcgattactactggacctggatccggcagtctcctggcaaaggcctggaatggatcggccacatctactactccggcaacaccaactacaaccccagcctgaagtcccggctgaccatctccatcgacaccagcaagacccagttctccctgaagctgtcctctgtgaccgccgctgataccgccatctactattgcgtgcgggacagagtgaccggcgcctttgatatttggggccagggcaccatggtcaccgtgtccagtgcttctaccaagggacccagcgtgttccctctggctccttgctccagatccacctccgagtctacagctgctctgggctgtctggtcaaggactacttccctgagcctgtgaccgtgtcctggaattctggcgctctgacatctggcgtgcacacctttccagctgtgctgcagtctagcggcctgtactctctgtcctccgtcgtgaccgtgccttcctctaactttggcacccagacctacacctgtaatgtggaccacaagccatccaacaccaaggtggacaagaccgtggaacggaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtctcacgaggatcccgaggtgcagttcaattggtacgtggacggcgtggaagtgcacaacgccaagaccaagcctagagaggaacagttcaactccaccttcagagtggtgtccgtgctgaccgtggtgcatcaggattggctgaacggcaaagagtacaagtgcaaggtgtccaacaagggcctgcctgctcctatcgaaaagaccatctctaagaccaaggggcagccccgggaacctcaggtttacacactgcctccaagccgggaagagatgaccaagaaccaggtgtccctgacctgtctcgtgaagggcttctacccctccgatatcgccgtggaatgggagtctaatggccagcctgagaacaactacaagaccacacctcctatgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggcggtagcggtggtggcggatctgaagttcagctggttgaatctggcggcggactggttcaaccaggcggatctctgagactgtcttgtgccgcctccggcttcaccttctcctcttacgctatgtcctgggtccgacaggccccaggcaaaggattggagtgggtgtcccagatctctcccgctggcggctacaccaattacgccgactctgtgaagggcagattcaccatctctgccgacacctccaagaacaccgcctacctgcagatgaactccctgagagccgaggacaccgccgtgtactattgtgctagaggcgagctgccctactaccggatgtccaaagtgatggacgtgtggggacagggaaccctcgtgacagtttctagcggtggcggaggtagcggaggcggtggaagcggcggaggcggaagtgatattcagatgacccagtctccttccagcctgtccgcttctgtgggcgatagagtgaccatcacctgtcgggcctctcagtacttctcctcctacctggcttggtatcagcagaagcctggcaaggcccctaagctgctgatctacggcgcctcctctagagctagcggcgtgccctctagattctccggatctggctctggcaccgactttaccctgacaatctccagcctgcagcctgaggacttcgccacctactactgccagcagtacctgggctctcctccaacctttggccagggaacaaaggtcgagatcaagcgctga

SEQ ID NO: 16 - DNA, 2121 bp, synthetic construct
  misc_feature 1..2121: note = Synthetic
  source 1..2121: mol_type = other DNA
caggttcagctgcaagagtctggccctggcctggtcaagccttccgaaacactgtctctgacctgcaccgtgtctggcggctctgtgtcctctggcgattactactggacctggatccggcagtctcctggcaaaggcctggaatggatcggccacatctactactccggcaacaccaactacaaccccagcctgaagtcccggctgaccatctccatcgacaccagcaagacccagttctccctgaagctgtcctctgtgaccgccgctgataccgccatctactattgcgtgcgggacagagtgaccggcgcctttgatatttggggccagggcaccatggtcaccgtgtccagtgcttctaccaagggacccagcgtgttccctctggctccttgctccagatccacctccgagtctacagctgctctgggctgtctggtcaaggactacttccctgagcctgtgaccgtgtcctggaattctggcgctctgacatctggcgtgcacacctttccagctgtgctgcagtctagcggcctgtactctctgtcctccgtcgtgaccgtgccttcctctaactttggcacccagacctacacctgtaatgtggaccacaagccatccaacaccaaggtggacaagaccgtggaacggaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtctcacgaggatcccgaggtgcagttcaattggtacgtggacggcgtggaagtgcacaacgccaagaccaagcctagagaggaacagttcaactccaccttcagagtggtgtccgtgctgaccgtggtgcatcaggattggctgaacggcaaagagtacaagtgcaaggtgtccaacaagggcctgcctgctcctatcgaaaagaccatctctaagaccaaggggcagccccgggaacctcaggtttacacactgcctccaagccgggaagagatgaccaagaaccaggtgtccctgacctgtctcgtgaagggcttctacccctccgatatcgccgtggaatgggagtctaatggccagcctgagaacaactacaagaccacacctcctatgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggcggtagcggtggtggcggatctgaagttcagctggttgaatctggcggcggactggttcaaccaggcggatctctgagactgtcttgtgccgcctccggcttcaccttctcctcttacgctatgtcctgggtccgacaggccccaggcaaaggattggagtgggtgtcccagatctctcccgctggcggctacaccaattacgccgactctgtgaagggcagattcaccatctctgccgacacctccaagaacaccgcctacctgcagatgaactccctgagagccgaggacaccgccgtgtactattgtgctagaggcgagctgccctacttccggatgtccaaagtgatggacgtgtggggacagggaaccctcgtgacagtttctagcggtggcggaggtagcggaggcggtggaagcggcggaggcggaagtgatattcagatgacccagtctccttccagcctgtccgcttctgtgggcgatagagtgaccatcacctgtcgggcctctcagttcctgtcctcctacctggcttggtatcagcagaagcctggcaaggcccctaagctgctgatctacggcgcttctgctagagcttccggcgtgccctccagattttctggctctggatctggcaccgactttaccctgacaatctccagcctgcagcctgaggacttcgccacctactactgccagcagtacctggcctctcctgccacatttggccagggaacaaaggtcgagatcaagcgctga

SEQ ID NO: 17 - DNA, 2121 bp, synthetic construct
  misc_feature 1..2121: note = Synthetic
  source 1..2121: mol_type = other DNA
caggttcagctgcaagagtctggccctggcctggtcaagccttccgaaacactgtctctgacctgcaccgtgtctggcggctctgtgtcctctggcgattactactggacctggatccggcagtctcctggcaaaggcctggaatggatcggccacatctactactccggcaacaccaactacaaccccagcctgaagtcccggctgaccatctccatcgacaccagcaagacccagttctccctgaagctgtcctctgtgaccgccgctgataccgccatctactattgcgtgcgggacagagtgaccggcgcctttgatatttggggccagggcaccatggtcaccgtgtccagtgcttctaccaagggacccagcgtgttccctctggctccttgctccagatccacctccgagtctacagctgctctgggctgtctggtcaaggactacttccctgagcctgtgaccgtgtcctggaattctggcgctctgacatctggcgtgcacacctttccagctgtgctgcagtctagcggcctgtactctctgtcctccgtcgtgaccgtgccttcctctaactttggcacccagacctacacctgtaatgtggaccacaagccatccaacaccaaggtggacaagaccgtggaacggaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtctcacgaggatcccgaggtgcagttcaattggtacgtggacggcgtggaagtgcacaacgccaagaccaagcctagagaggaacagttcaactccaccttcagagtggtgtccgtgctgaccgtggtgcatcaggattggctgaacggcaaagagtacaagtgcaaggtgtccaacaagggcctgcctgctcctatcgaaaagaccatctctaagaccaaggggcagccccgggaacctcaggtttacacactgcctccaagccgggaagagatgaccaagaaccaggtgtccctgacctgtctcgtgaagggcttctacccctccgatatcgccgtggaatgggagtctaatggccagcctgagaacaactacaagaccacacctcctatgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggcggtagcggtggtggcggatctgaagttcagctggttgaatctggcggcggactggttcaaccaggcggatctctgagactgtcttgtgccgcctccggcttcaccttctcctcttacgctatgtcctgggtccgacaggccccaggcaaaggattggagtgggtgtcccagatctctcccgctggcggctacaccaattacgccgactctgtgaagggcagattcaccatctctgccgacacctccaagaacaccgcctacctgcagatgaactccctgagagccgaggacaccgccgtgtactattgtgctagaggcgagctgccctactaccagatgtccaaagtgatggacgtgtggggacagggaaccctcgtgacagtttctagcggtggcggaggtagcggaggcggtggaagcggcggaggcggaagtgatattcagatgacccagtctccttccagcctgtccgcttctgtgggcgatagagtgaccatcacctgtcgggcctctcagtacttctcttcctatctggcatggtatcagcagaagcctggcaaggcccctaagctgctgatctacggcgcttctgctagagcttccggcgtgccctccagattttctggctctggatctggcaccgactttaccctgacaatctccagcctgcagcctgaggacttcgccacctactactgccagcagtacctggcctctcctgccacatttggccagggaacaaaggtcgagatcaagcgctga

SEQ ID NO: 18 - DNA, 2121 bp, synthetic construct
  misc_feature 1..2121: note = Synthetic
  source 1..2121: mol_type = other DNA
caggttcagctgcaagagtctggccctggcctggtcaagccttccgaaacactgtctctgacctgcaccgtgtctggcggctctgtgtcctctggcgattactactggacctggatccggcagtctcctggcaaaggcctggaatggatcggccacatctactactccggcaacaccaactacaaccccagcctgaagtcccggctgaccatctccatcgacaccagcaagacccagttctccctgaagctgtcctctgtgaccgccgctgataccgccatctactattgcgtgcgggacagagtgaccggcgcctttgatatttggggccagggcaccatggtcaccgtgtccagtgcttctaccaagggacccagcgtgttccctctggctccttgctccagatccacctccgagtctacagctgctctgggctgtctggtcaaggactacttccctgagcctgtgaccgtgtcctggaattctggcgctctgacatctggcgtgcacacctttccagctgtgctgcagtctagcggcctgtactctctgtcctccgtcgtgaccgtgccttcctctaactttggcacccagacctacacctgtaatgtggaccacaagccatccaacaccaaggtggacaagaccgtggaacggaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtctcacgaggatcccgaggtgcagttcaattggtacgtggacggcgtggaagtgcacaacgccaagaccaagcctagagaggaacagttcaactccaccttcagagtggtgtccgtgctgaccgtggtgcatcaggattggctgaacggcaaagagtacaagtgcaaggtgtccaacaagggcctgcctgctcctatcgaaaagaccatctctaagaccaaggggcagccccgggaacctcaggtttacacactgcctccaagccgggaagagatgaccaagaaccaggtgtccctgacctgtctcgtgaagggcttctacccctccgatatcgccgtggaatgggagtctaatggccagcctgagaacaactacaagaccacacctcctatgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggcggtagcggtggtggcggatctgaagttcagctggttgaatctggcggcggactggttcaaccaggcggatctctgagactgtcttgtgccgcctccggcttcaccttctcctcttacgctatgtcctgggtccgacaggccccaggcaaaggattggagtgggtgtcccagatctctcccgctggcggctacaccaattacgccgactctgtgaagggcagattcaccatctctgccgacacctccaagaacaccgcctacctgcagatgaactccctgagagccgaggacaccgccgtgtactattgtgctagaggcgagctgccattcttccggatgtcccaagtgatggacgtgtggggacagggaaccctcgtgacagtttctagcggtggcggaggtagcggaggcggtggaagcggcggaggcggaagtgatattcagatgacccagtctccttccagcctgtccgcttctgtgggcgatagagtgaccatcacctgtcgggcctctcagttcctgtctagttatctggcatggtatcagcagaagcccggcaaggctcccaagctgttgatctacggcgcctctgctagagcttccggcgtgccatctagattctccggctctggctctggcaccgactttaccctgacaatctccagcctgcagcctgaggacttcgccacctactactgccagcagtacctggcctctcctgccacatttggccagggaacaaaggtcgagatcaagcgctga

SEQ ID NO: 19 - DNA, 2121 bp, synthetic construct
  misc_feature 1..2121: note = Synthetic
  source 1..2121: mol_type = other DNA
caggttcagctgcaagagtctggccctggcctggtcaagccttccgaaacactgtctctgacctgcaccgtgtctggcggctctgtgtcctctggcgattactactggacctggatccggcagtctcctggcaaaggcctggaatggatcggccacatctactactccggcaacaccaactacaaccccagcctgaagtcccggctgaccatctccatcgacaccagcaagacccagttctccctgaagctgtcctctgtgaccgccgctgataccgccatctactattgcgtgcgggacagagtgaccggcgcctttgatatttggggccagggcaccatggtcaccgtgtccagtgcttctaccaagggacccagcgtgttccctctggctccttgctccagatccacctccgagtctacagctgctctgggctgtctggtcaaggactacttccctgagcctgtgaccgtgtcctggaattctggcgctctgacatctggcgtgcacacctttccagctgtgctgcagtctagcggcctgtactctctgtcctccgtcgtgaccgtgccttcctctaactttggcacccagacctacacctgtaatgtggaccacaagccatccaacaccaaggtggacaagaccgtggaacggaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtctcacgaggatcccgaggtgcagttcaattggtacgtggacggcgtggaagtgcacaacgccaagaccaagcctagagaggaacagttcaactccaccttcagagtggtgtccgtgctgaccgtggtgcatcaggattggctgaacggcaaagagtacaagtgcaaggtgtccaacaagggcctgcctgctcctatcgaaaagaccatctctaagaccaaggggcagccccgggaacctcaggtttacacactgcctccaagccgggaagagatgaccaagaaccaggtgtccctgacctgtctcgtgaagggcttctacccctccgatatcgccgtggaatgggagtctaatggccagcctgagaacaactacaagaccacacctcctatgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggcggtagcggtggtggcggatctgaagttcagctggttgaatctggcggcggactggttcaaccaggcggatctctgagactgtcttgtgccgcctccggcttcaccttctcctcttacgctatgtcctgggtccgacaggccccaggcaaaggattggagtgggtgtcccagatctctcccgctggcggctacaccaattacgccgactctgtgaagggcagattcaccatctctgccgacacctccaagaacaccgcctacctgcagatgaactccctgagagccgaggacaccgccgtgtactattgtgctagaggcgagctgccctacttccagatgtccaaagtgatggacgtgtggggacagggaaccctcgtgacagtttctagcggtggcggaggtagcggaggcggtggaagcggcggaggcggaagtgatattcagatgacccagtctccttccagcctgtccgcttctgtgggcgatagagtgaccatcacctgtcgggcctctcagtacttctactcctatctggcttggtatcagcagaagcctggcaaggcccctaagctgctgatctacggcgcttctgctagagcttccggcgtgccctccagattttctggctctggatctggcaccgactttaccctgacaatctccagcctgcagcctgaggacttcgccacctactactgccagcagtacctggcctctcctgccacatttggccagggaacaaaggtcgagatcaagcgctga

SEQ ID NO: 20 - DNA, 2133 bp, synthetic construct
  misc_feature 1..2133: note = Synthetic
  source 1..2133: mol_type = other DNA
caggttcagctgcaagagtctggccctggcctggtcaagccttccgaaacactgtctctgacctgcaccgtgtctggcggctctgtgtcctctggcgattactactggacctggatccggcagtctcctggcaaaggcctggaatggatcggccacatctactactccggcaacaccaactacaaccccagcctgaagtcccggctgaccatctccatcgacaccagcaagacccagttctccctgaagctgtcctctgtgaccgccgctgataccgccatctactattgcgtgcgggacagagtgaccggcgcctttgatatttggggccagggcaccatggtcaccgtgtccagtgcttctaccaagggacccagcgtgttccctctggctccttccagcaagtctacctctggcggaacagctgctctgggctgtctggtcaaggactacttccctgagcctgtgaccgtgtcctggaattctggcgctctgacatctggcgtgcacacctttccagctgtgctgcagtctagcggcctgtactctctgtcctccgtcgtgaccgtgccttctagctctctgggcacccagacctacatctgcaatgtgaaccacaagccatccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggctaagggccagcctcgggaaccccaggtttacacattgcctccatctcgggacgagctgaccaagaaccaggtgtccctgacctgtctcgtgaagggcttctacccctccgatatcgccgtggaatgggagtccaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggtggatctgaagtgcagctggttgaaagtggcggcggattggttcagcctggcggatctctgagactgtcttgtgccgcctccggctttaccttctcctcctacgctatgtcctgggtccgacaggctcccggaaaaggacttgaatgggtgtcccagatctcccctgctggcggctacaccaattacgccgactctgtgaagggcagattcaccatctctgccgacacctccaagaacaccgcctacctgcagatgaactccctgagagccgaggacaccgccgtgtactattgtgctagaggcgagctgccctacttccggatgtccaaagtgatggacgtgtggggacagggaaccctcgtgacagtttctagtggtggcggaggaagcggcggaggcggttctggcggtggtggatctgatatccagatgacccagtctcctagcagcctgtctgcctctgtgggcgatagagtgaccatcacctgtcgggcctctcagttcctgtccagctacctggcttggtatcagcagaagcctggcaaggcccctaagctgctgatctacggcgcttctgctagagcttccggcgtgccctccagattttctggctctggatctggcaccgactttaccctgacaatctccagcctgcagcctgaggacttcgccacctactactgccagcagtacctggcctctcctgccacatttggccagggaacaaaggtcgagatcaagcgctga

SEQ ID NO: 21 - DNA, 2118 bp, synthetic construct
  misc_feature 1..2118: note = Synthetic
  source 1..2118: mol_type = other DNA
caggttcagctgcaagagtctggccctggcctggtcaagccttccgaaacactgtctctgacctgcaccgtgtctggcggctctgtgtcctctggcgattactactggacctggatccggcagtctcctggcaaaggcctggaatggatcggccacatctactactccggcaacaccaactacaaccccagcctgaagtcccggctgaccatctccatcgacaccagcaagacccagttctccctgaagctgtcctctgtgaccgccgctgataccgccatctactattgcgtgcgggacagagtgaccggcgcctttgatatttggggccagggcaccatggtcaccgtgtccagtgcttctaccaagggacccagcgtgttccctctggctccttccagcaagtctacctctggcggaacagctgctctgggctgtctggtcaaggactacttccctgagcctgtgaccgtgtcctggaattctggcgctctgacatctggcgtgcacacctttccagctgtgctgcagtctagcggcctgtactctctgtcctccgtcgtgaccgtgccttctagctctctgggcacccagacctacatctgcaatgtgaaccacaagccatccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggctaagggccagcctcgggaaccccaggtttacacattgcctccatctcgggacgagctgaccaagaaccaggtgtccctgacctgtctcgtgaagggcttctacccctccgatatcgccgtggaatgggagtccaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggtggatctgaagtgcagctggttgaaagtggcggcggattggttcagcctggcggatctctgagactgtcttgtgccgcctccggctttaccttctcctcctacgctatgtcctgggtccgacaggctcccggaaaaggacttgaatgggtgtcccagatctcccctgctggcggctacaccaattacgccgactctgtgaagggcagattcaccatctctgccgacacctccaagaacaccgcctacctgcagatgaactccctgagagccgaggacaccgccgtgtactattgtgctagaggcgagctgccctacttccggatgtccaaagtgatggacgtgtggggacagggaaccctcgtgacagtgtctagcggagatggatctagtggtggcggaggcgcttccgacatccagatgacacagtctccctccagcctgtctgcctctgtgggcgatagagtgaccatcacctgtcgggcctctcagttcctgtccagctacctggcttggtatcagcagaagcctggcaaggcccctaagctgctgatctacggcgcttctgctagagcttccggcgtgccctccagattttctggctctggatctggcaccgactttaccctgacaatcagcagcctgcagcctgaggacttcgccacctactactgccagcagtacctggcctctcctgccacatttggccagggaacaaaggtcgagatcaagtga

SEQ ID NO: 22 - DNA, 2145 bp, synthetic construct
  misc_feature 1..2145: note = Synthetic
  source 1..2145: mol_type = other DNA
caggttcagctgcaagagtctggccctggcctggtcaagccttccgaaacactgtctctgacctgcaccgtgtctggcggctctgtgtcctctggcgattactactggacctggatccggcagtctcctggcaaaggcctggaatggatcggccacatctactactccggcaacaccaactacaaccccagcctgaagtcccggctgaccatctccatcgacaccagcaagacccagttctccctgaagctgtcctctgtgaccgccgctgataccgccatctactattgcgtgcgggacagagtgaccggcgcctttgatatttggggccagggcaccatggtcaccgtgtccagtgcttctaccaagggacccagcgtgttccctctggctccttccagcaagtctacctctggcggaacagctgctctgggctgtctggtcaaggactacttccctgagcctgtgaccgtgtcctggaattctggcgctctgacatctggcgtgcacacctttccagctgtgctgcagtctagcggcctgtactctctgtcctccgtcgtgaccgtgccttctagctctctgggcacccagacctacatctgcaatgtgaaccacaagccatccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggctaagggccagcctcgggaaccccaggtttacacattgcctccatctcgggacgagctgaccaagaaccaggtgtccctgacctgtctcgtgaagggcttctacccctccgatatcgccgtggaatgggagtccaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggtggatctgaagtgcagctggttgaaagtggcggcggattggttcagcctggcggatctctgagactgtcttgtgccgcctccggctttaccttctcctcctacgctatgtcctgggtccgacaggctcccggaaaaggacttgaatgggtgtcccagatctcccctgctggcggctacaccaattacgccgactctgtgaagggcagattcaccatctctgccgacacctccaagaacaccgcctacctgcagatgaactccctgagagccgaggacaccgccgtgtactattgtgctagaggcgagctgccctacttccggatgtccaaagtgatggacgtgtggggacagggaaccctcgtgacagtttctagtggtggcggaggaagcggcggaggcggttctggcggtggtggctctggcggtggcggatctgatatccagatgacccagtctcctagcagcctgtctgcctctgtgggcgatagagtgaccatcacctgtcgggcctctcagttcctgtccagctacctggcttggtatcagcagaagcctggcaaggcccctaagctgctgatctacggcgcttctgctagagcttccggcgtgccctccagattctctggctctggatctggcaccgactttaccctgacaatctccagcctgcagcctgaggacttcgccacctactactgccagcagtacctggcctctcctgccacatttggccagggaacaaaggtcgagatcaagtga

SEQ ID NO: 23 - DNA, 2130 bp, synthetic construct
  misc_feature 1..2130: note = Synthetic
  source 1..2130: mol_type = other DNA
caggttcagctgcaagagtctggccctggcctggtcaagccttccgaaacactgtctctgacctgcaccgtgtctggcggctctgtgtcctctggcgattactactggacctggatccggcagtctcctggcaaaggcctggaatggatcggccacatctactactccggcaacaccaactacaaccccagcctgaagtcccggctgaccatctccatcgacaccagcaagacccagttctccctgaagctgtcctctgtgaccgccgctgataccgccatctactattgcgtgcgggacagagtgaccggcgcctttgatatttggggccagggcaccatggtcaccgtgtccagtgcttctaccaagggacccagcgtgttccctctggctccttccagcaagtctacctctggcggaacagctgctctgggctgtctggtcaaggactacttccctgagcctgtgaccgtgtcctggaattctggcgctctgacatctggcgtgcacacctttccagctgtgctgcagtctagcggcctgtactctctgtcctccgtcgtgaccgtgccttctagctctctgggcacccagacctacatctgcaatgtgaaccacaagccatccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggctaagggccagcctcgggaaccccaggtttacacattgcctccatctcgggacgagctgaccaagaaccaggtgtccctgacctgtctcgtgaagggcttctacccctccgatatcgccgtggaatgggagtccaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggtggatctgatatccagatgacccagtctcctagcagcctgtctgcctctgtgggcgatagagtgaccatcacctgtcgggcctctcagttcctgtccagctacctggcttggtatcagcagaagcctggcaaggcccctaagctgctgatctacggcgcttctgctagagcttccggcgtgccctccagattttctggctctggatctggcaccgactttaccctgacaatctccagcctgcagcctgaggacttcgccacctactactgccagcagtacctggcctctcctgccacatttggccagggaacaaaggtggaaatcaaaggtggcggcggtagtggtggcggaggaagcggcggaggcggctctgaagttcagcttgttgaatctggcggcggactggttcagcctggcggatctctgagactgtcttgtgccgccagcggcttcaccttctcctcttacgctatgtcctgggtccgacaggccccaggcaaaggattggagtgggtgtcccagatctctcctgctggcggctacaccaattacgccgactctgtgaagggcagattcaccatctctgccgacacctccaagaacaccgcctacctgcagatgaactccctgagagccgaggacaccgccgtgtactattgtgctagaggcgagctgccctacttccggatgtccaaagtgatggacgtgtggggacagggaaccctcgtgacagtgtcctcttga

SEQ ID NO: 24 - DNA, 2130 bp, synthetic construct
  misc_feature 1..2130: note = Synthetic
  source 1..2130: mol_type = other DNA
caggttcagctgcaagagtctggccctggcctggtcaagccttccgaaacactgtctctgacctgcaccgtgtctggcggctctgtgtcctctggcgattactactggacctggatccggcagtctcctggcaaaggcctggaatggatcggccacatctactactccggcaacaccaactacaaccccagcctgaagtcccggctgaccatctccatcgacaccagcaagacccagttctccctgaagctgtcctctgtgaccgccgctgataccgccatctactattgcgtgcgggacagagtgaccggcgcctttgatatttggggccagggcaccatggtcaccgtgtccagtgcttctaccaagggacccagcgtgttccctctggctccttccagcaagtctacctctggcggaacagctgctctgggctgtctggtcaaggactacttccctgagcctgtgaccgtgtcctggaattctggcgctctgacatctggcgtgcacacctttccagctgtgctgcagtctagcggcctgtactctctgtcctccgtcgtgaccgtgccttctagctctctgggcacccagacctacatctgcaatgtgaaccacaagccatccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggctaagggccagcctcgggaaccccaggtttacacattgcctccatctcgggacgagctgaccaagaaccaggtgtccctgacctgtctcgtgaagggcttctacccctccgatatcgccgtggaatgggagtccaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggtggatctgaagtgcagctgttggaaagtggcggcggattggttcagcctggcggatctctgagactgtcttgtgccgcctccggctttaccttctcctcctacgctatgtcctgggtccgacaggctcccggaaaaggacttgaatgggtgtcccagatctcccctgctggcggctacaccaattacgccgactctgtgaagggcagattcaccatctctcgggacaactccaagaacaccctgtacctgcagatgaactccctgagagccgaggacaccgccgtgtactattgtgctagaggcgagctgccctacttccggatgtccaaagtgatggacgtgtggggacagggaaccctcgtgacagtttctagtggtggcggaggaagcggcggaggcggttctggcggtggcggatctgaaattgtgctgacccagtctccaggcacactcagtttgagccctggcgagagagctaccctgagctgtagagcctctcagttcctgtccagctacctggcttggtatcagcagaagccaggacaggcccctcggctgttgatctatggcgcttctgctagagccagcggcatccctgatagattctccggctctggctctggcaccgacttcaccctgacaatctcccggctggaacctgaggacttcgctgtgtactactgccagcagtacctggcctctcctgccacatttggccagggaacaaaggtcgagatcaagtga

SEQ ID NO: 25 - DNA, 642 bp, synthetic construct
  misc_feature 1..642: note = Synthetic
  source 1..642: mol_type = other DNA
gacatccagatgacccagtctccatcatccctgtcggcctcagtgggcgacagagtgaccatcacttgtcaagcctcccaagacattagcaactacctgaactggtaccagcagaagcccggaaaggccccgaagctgctcatctatgacgcttccaaccttgagactggagtgccttcgcgcttctccggctccgggagcggtaccgatttcaccttcaccatctcctccctgcaacccgaggacattgcgacttacttctgccaacatttcgatcacctccctctcgcgttcggcggcggaactaaggtcgagattaagcggaccgtggctgccccgtccgtgttcatcttcccgccgtccgatgaacagctgaagtccggtaccgcatcagtcgtgtgcttgctgaacaacttctacccccgggaagccaaggtccagtggaaagtggacaatgcgctgcagtcgggaaactcgcaggaatccgtgaccgaacaggattcgaaggacagcacatacagcctgtcatccaccctcacgctgtcgaaggccgactacgagaagcacaaagtgtacgcctgcgaagtgacccaccaagggcttagcagccctgtgaccaagtccttcaaccgcggagagtgc

SEQ ID NO: 26 - DNA, 15 bp, synthetic construct
  misc_feature 1..15: note = Synthetic
  source 1..15: mol_type = other DNA
ggcggtggcggatcc

SEQ ID NO: 27 - DNA, 15 bp, synthetic construct
  misc_feature 1..15: note = Synthetic
  source 1..15: mol_type = other DNA
ggcggcgggggcagc

SEQ ID NO: 28 - DNA, 15 bp, synthetic construct
  misc_feature 1..15: note = Synthetic
  source 1..15: mol_type = other DNA
ggcggcggaggatct

SEQ ID NO: 29 - DNA, 15 bp, synthetic construct
  misc_feature 1..15: note = Synthetic
  source 1..15: mol_type = other DNA
ggcggaggcggtagc

SEQ ID NO: 30 - DNA, 15 bp, synthetic construct
  misc_feature 1..15: note = Synthetic
  source 1..15: mol_type = other DNA
ggtggtggcggatct

SEQ ID NO: 31 - DNA, 15 bp, synthetic construct
  misc_feature 1..15: note = Synthetic
  source 1..15: mol_type = other DNA
ggcggcggaggatct

SEQ ID NO: 32 - DNA, 15 bp, synthetic construct
  misc_feature 1..15: note = Synthetic
  source 1..15: mol_type = other DNA
ggcggaggtggaagc

SEQ ID NO: 33 - DNA, 15 bp, synthetic construct
  misc_feature 1..15: note = Synthetic
  source 1..15: mol_type = other DNA
ggaggcggtggatct

SEQ ID NO: 34 - DNA, 15 bp, synthetic construct
  misc_feature 1..15: note = Synthetic
  source 1..15: mol_type = other DNA
ggtggcggaggaagc

SEQ ID NO: 35 - DNA, 15 bp, synthetic construct
  misc_feature 1..15: note = Synthetic
  source 1..15: mol_type = other DNA
ggcggaggcggttct

SEQ ID NO: 36 - DNA, 15 bp, synthetic construct
  misc_feature 1..15: note = Synthetic
  source 1..15: mol_type = other DNA
ggcggtggcggatct

SEQ ID NO: 37 - DNA, 15 bp, synthetic construct
  misc_feature 1..15: note = Synthetic
  source 1..15: mol_type = other DNA
ggcggtggcggatcc

SEQ ID NO: 38 - DNA, 15 bp, synthetic construct
  misc_feature 1..15: note = Synthetic
  source 1..15: mol_type = other DNA
ggcggcgggggcagc

SEQ ID NO: 39 - protein, 709 aa, synthetic construct
  REGION 1..709: note = Synthetic
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTFSSYAMSWVRQAPGKGLEWVSQISPAGGYTNYADSVKGRFTISRDNSKNTLYLQMNSLRAEDTAVYYCARGELPYHRMSKVMDVWGQGTLVTVSSGGGGSGGGGSGGGGSEIVLTQSPGTLSLSPGERATLSCRASQFLSSYLAWYQQKPGQAPRLLIYGASARASGIPDRFSGSGSGTDFTLTISRLEPEDFAVYYCQQYLASPATFGQGTKVEIK

SEQ ID NO: 40 - DNA, 2127 bp, synthetic construct
  misc_feature 1..2127: note = Synthetic
  source 1..2127: mol_type = other DNA
caggttcagctgcaagagtctggccctggcctggtcaagccttccgaaacactgtctctgacctgcaccgtgtctggcggctctgtgtcctctggcgattactactggacctggatccggcagtctcctggcaaaggcctggaatggatcggccacatctactactccggcaacaccaactacaaccccagcctgaagtcccggctgaccatctccatcgacaccagcaagacccagttctccctgaagctgtcctctgtgaccgccgctgataccgccatctactattgcgtgcgggacagagtgaccggcgcctttgatatttggggccagggcaccatggtcaccgtgtccagtgcttctaccaagggacccagcgtgttccctctggctccttccagcaagtctacctctggcggaacagctgctctgggctgtctggtcaaggactacttccctgagcctgtgaccgtgtcctggaattctggcgctctgacatctggcgtgcacacctttccagctgtgctgcagtctagcggcctgtactctctgtcctccgtcgtgaccgtgccttctagctctctgggcacccagacctacatctgcaatgtgaaccacaagccatccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggctaagggccagcctcgggaaccccaggtttacacattgcctccatctcgggacgagctgaccaagaaccaggtgtccctgacctgtctcgtgaagggcttctacccctccgatatcgccgtggaatgggagtccaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggtggatctgaagtgcagctgttggaaagtggcggcggattggttcagcctggcggatctctgagactgtcttgtgccgcctccggctttaccttctcctcctacgctatgtcctgggtccgacaggctcccggaaaaggacttgaatgggtgtcccagatctcccctgctggcggctacaccaattacgccgactctgtgaagggcagattcaccatctctcgggacaactccaagaacaccctgtacctgcagatgaactccctgagagccgaggacaccgccgtgtactattgtgctagaggcgagctgccctaccaccggatgtccaaagtgatggatgtgtggggacagggaaccctcgtgacagtctctagtggtggcggaggaagcggcggaggcggttctggcggtggcggatctgaaattgtgctgacccagtctccaggcacactcagtttgagccctggcgagagagctaccctgagctgtagagcctctcagttcctgtccagctacctggcttggtatcagcagaagccaggacaggcccctcggctgttgatctatggcgcttctgctagagccagcggcatccctgatagattctccggctctggctctggcaccgacttcaccctgacaatctcccggctggaacctgaggacttcgctgtgtactactgccagcagtacctggcctctcctgccacatttggccagggaacaaaggtcgagatcaag

SEQ ID NO: 41 - protein, 453 aa, synthetic construct
  REGION 1..453: note = Synthetic
EVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPYFRMSKVMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 453 aa, synthetic construct
  REGION 1..453: note = Synthetic
EVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPYYQMSKVMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 453 aa, synthetic construct
  REGION 1..453: note = Synthetic
EVQLVESGGGLVQPGGSLRLSCAASGFTFSSYAMSWVRQAPGKGLEWVSQISPAGGYTNYADSVKGRFTISADTSKNTAYLQMNSLRAEDTAVYYCARGELPFFRMSQVMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 453 aa, synthetic construct; identical to SEQ ID NO: 41
  REGION 1..453: note = Synthetic

SEQ ID NO: 45 - protein, 453 aa, synthetic construct
  REGION 1..453: note = Synthetic
EVQLLESGGGLVQPGGSLRLSCAASGFTFSSYAMSWVRQAPGKGLEWVSQISPAGGYTNYADSVKGRFTISRDNSKNTLYLQMNSLRAEDTAVYYCARGELPYFRMSKVMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 453 aa, synthetic construct
  REGION 1..453: note = Synthetic
EVQLLESGGGLVQPGGSLRLSCAASGFTFSSYAMSWVRQAPGKGLEWVSQISPAGGYTNYADSVKGRFTISRDNSKNTLYLQMNSLRAEDTAVYYCARGELPYYQMSKVMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 453 aa, synthetic construct
  REGION 1..453: note = Synthetic
EVQLLESGGGLVQPGGSLRLSCAASGFTFSSYAMSWVRQAPGKGLEWVSQISPAGGYTNYADSVKGRFTISRDNSKNTLYLQMNSLRAEDTAVYYCARGELPFFRMSQVMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 214 aa, synthetic construct
  REGION 1..214: note = Synthetic
DIQMTQSPSSLSASVGDRVTITCRASQFLSSYLAWYQQKPGKAPKLLIYGASARASGVPSRFSGSGSGTDFTLTISSLQPEDFATYYCQQYLASPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 214 aa, synthetic construct
  REGION 1..214: note = Synthetic
DIQMTQSPSSLSASVGDRVTITCRASQYFSSYLAWYQQKPGKAPKLLIYGASARASGVPSRFSGSGSGTDFTLTISSLQPEDFATYYCQQYLASPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 214 aa, synthetic construct; identical to SEQ ID NO: 48
  REGION 1..214: note = Synthetic

SEQ ID NO: 51 - protein, 214 aa, synthetic construct
  REGION 1..214: note = Synthetic
DIQMTQSPSSLSASVGDRVTITCRASQFLSSFLAWYQQKPGKAPKLLIYGASARASGVPSRFSGSGSGTDFTLTISSLQPEDFATYYCQQYLASPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 214 aa, synthetic construct
  REGION 1..214: note = Synthetic
EIVLTQSPGTLSLSPGERATLSCRASQFLSSYLAWYQQKPGQAPRLLIYGASARASGIPDRFSGSGSGTDFTLTISRLEPEDFAVYYCQQYLASPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 214 aa, synthetic construct; identical to SEQ ID NO: 52
  REGION 1..214: note = Synthetic

SEQ ID NO: 54 - protein, 214 aa, synthetic construct; identical to SEQ ID NO: 52
  REGION 1..214: note = Synthetic

SEQ ID NO: 55 - DNA, 1359 bp, synthetic construct
  misc_feature 1..1359: note = Synthetic
  source 1..1359: mol_type = other DNA
gaggtgcagctggtggagagcggcggcggcctggtgcagcccggcggcagcctgaggctgagctgcgccgccagcggcttcaccttcagcagctacgccatgagctgggtgaggcaggcccccggcaagggcctggagtgggtgagccagatcagccccgccggcggctacaccaactacgccgacagcgtgaagggcaggttcaccatcagcgccgacaccagcaagaacaccgcctacctgcagatgaacagcctgagggccgaggacaccgccgtgtactactgcgccaggggcgagctgccctacttcaggatgagcaaggtgatggacgtgtggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 56 - DNA, 1359 bp, synthetic construct
  misc_feature 1..1359: note = Synthetic
  source 1..1359: mol_type = other DNA
gaggtgcagctggtggagagcggcggcggcctggtgcagcccggcggcagcctgaggctgagctgcgccgccagcggcttcaccttcagcagctacgccatgagctgggtgaggcaggcccccggcaagggcctggagtgggtgagccagatcagccccgccggcggctacaccaactacgccgacagcgtgaagggcaggttcaccatcagcgccgacaccagcaagaacaccgcctacctgcagatgaacagcctgagggccgaggacaccgccgtgtactactgcgccaggggcgagctgccctactaccagatgagcaaggtgatggacgtgtggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 57 - DNA, 1359 bp, synthetic construct
  misc_feature 1..1359: note = Synthetic
  source 1..1359: mol_type = other DNA
gaggtgcagctggtggagagcggcggcggcctggtgcagcccggcggcagcctgaggctgagctgcgccgccagcggcttcaccttcagcagctacgccatgagctgggtgaggcaggcccccggcaagggcctggagtgggtgagccagatcagccccgccggcggctacaccaactacgccgacagcgtgaagggcaggttcaccatcagcgccgacaccagcaagaacaccgcctacctgcagatgaacagcctgagggccgaggacaccgccgtgtactactgcgccaggggcgagctgcccttcttcaggatgagccaggtgatggacgtgtggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 58 - DNA, 1359 bp, synthetic construct; identical to SEQ ID NO: 55
  misc_feature 1..1359: note = Synthetic
  source 1..1359: mol_type = other DNA

SEQ ID NO: 59 - DNA, 1359 bp, synthetic construct
  misc_feature 1..1359: note = Synthetic
  source 1..1359: mol_type = other DNA
gaggtgcagctgctggagagcggcggcggcctggtgcagcccggcggcagcctgaggctgagctgcgccgccagcggcttcaccttcagcagctacgccatgagctgggtgaggcaggcccccggcaagggcctggagtgggtgagccagatcagccccgccggcggctacaccaactacgccgacagcgtgaagggcaggttcaccatcagcagggacaacagcaagaacaccctgtacctgcagatgaacagcctgagggccgaggacaccgccgtgtactactgcgccaggggcgagctgccctacttcaggatgagcaaggtgatggacgtgtggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 60 - DNA, 1359 bp, synthetic construct
  misc_feature 1..1359: note = Synthetic
  source 1..1359: mol_type = other DNA
gaggtgcagctgctggagagcggcggcggcctggtgcagcccggcggcagcctgaggctgagctgcgccgccagcggcttcaccttcagcagctacgccatgagctgggtgaggcaggcccccggcaagggcctggagtgggtgagccagatcagccccgccggcggctacaccaactacgccgacagcgtgaagggcaggttcaccatcagcagggacaacagcaagaacaccctgtacctgcagatgaacagcctgagggccgaggacaccgccgtgtactactgcgccaggggcgagctgccctactaccagatgagcaaggtgatggacgtgtggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 61 - DNA, 1359 bp, synthetic construct
  misc_feature 1..1359: note = Synthetic
  source 1..1359: mol_type = other DNA
gaggtgcagctgctggagagcggcggcggcctggtgcagcccggcggcagcctgaggctgagctgcgccgccagcggcttcaccttcagcagctacgccatgagctgggtgaggcaggcccccggcaagggcctggagtgggtgagccagatcagccccgccggcggctacaccaactacgccgacagcgtgaagggcaggttcaccatcagcagggacaacagcaagaacaccctgtacctgcagatgaacagcctgagggccgaggacaccgccgtgtactactgcgccaggggcgagctgcccttcttcaggatgagccaggtgatggacgtgtggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 62 - DNA, 642 bp, synthetic construct
  misc_feature 1..642: note = Synthetic
  source 1..642: mol_type = other DNA
gacatccagatgacccagagccccagcagcctgagcgccagcgtgggcgacagggtgaccatcacctgcagggccagccagttcctgagcagctacctggcctggtaccagcagaagcccggcaaggcccccaagctgctgatctacggcgccagcgccagggccagcggcgtgcccagcaggttcagcggcagcggcagcggcaccgacttcaccctgaccatcagcagcctgcagcccgaggacttcgccacctactactgccagcagtacctggccagccccgccaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgc

SEQ ID NO: 63 - DNA, 642 bp, synthetic construct
  misc_feature 1..642: note = Synthetic
  source 1..642: mol_type = other DNA
gacatccagatgacccagagccccagcagcctgagcgccagcgtgggcgacagggtgaccatcacctgcagggccagccagtacttcagcagctacctggcctggtaccagcagaagcccggcaaggcccccaagctgctgatctacggcgccagcgccagggccagcggcgtgcccagcaggttcagcggcagcggcagcggcaccgacttcaccctgaccatcagcagcctgcagcccgaggacttcgccacctactactgccagcagtacctggccagccccgccaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgc

SEQ ID NO: 64 - DNA, 642 bp, synthetic construct; identical to SEQ ID NO: 62
  misc_feature 1..642: note = Synthetic
  source 1..642: mol_type = other DNA

SEQ ID NO: 65 - DNA, 642 bp, synthetic construct
  misc_feature 1..642: note = Synthetic
  source 1..642: mol_type = other DNA
gacatccagatgacccagagccccagcagcctgagcgccagcgtgggcgacagggtgaccatcacctgcagggccagccagttcctgagcagcttcctggcctggtaccagcagaagcccggcaaggcccccaagctgctgatctacggcgccagcgccagggccagcggcgtgcccagcaggttcagcggcagcggcagcggcaccgacttcaccctgaccatcagcagcctgcagcccgaggacttcgccacctactactgccagcagtacctggccagccccgccaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgc

SEQ ID NO: 66 - DNA, 642 bp, synthetic construct
  misc_feature 1..642: note = Synthetic
  source 1..642: mol_type = other DNA
gagatcgtgctgacccagagccccggcaccctgagcctgagccccggcgagagggccaccctgagctgcagggccagccagttcctgagcagctacctggcctggtaccagcagaagcccggccaggcccccaggctgctgatctacggcgccagcgccagggccagcggcatccccgacaggttcagcggcagcggcagcggcaccgacttcaccctgaccatcagcaggctggagcccgaggacttcgccgtgtactactgccagcagtacctggccagccccgccaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgc

SEQ ID NO: 67 - DNA, 642 bp, synthetic construct; identical to SEQ ID NO: 66
  misc_feature 1..642: note = Synthetic
  source 1..642: mol_type = other DNA

SEQ ID NO: 68 - DNA, 642 bp, synthetic construct; identical to SEQ ID NO: 66
  misc_feature 1..642: note = Synthetic
  source 1..642: mol_type = other DNA

SEQ ID NO: 69 - protein, 119 aa, synthetic construct [sequence omitted]

SEQ ID NO: 70 - protein, 107 aa, synthetic construct [sequence omitted]

SEQ ID NO: 71 - protein, 7 aa, synthetic construct
  REGION 1..7: note = Synthetic: Anti-EGFR VH CDR1 AA Seq
SGDYYWT

SEQ ID NO: 72 - protein, 15 aa, synthetic construct
  REGION 1..15: note = Synthetic: Anti-EGFR VH CDR2 AA Seq
HIYYSGNTNYNPSLK

SEQ ID NO: 73 - protein, 8 aa, synthetic construct
  REGION 1..8: note = Synthetic: Anti-EGFR VH CDR3 AA Seq
RVTGAFDI

SEQ ID NO: 74 - protein, 10 aa, synthetic construct
  REGION 1..10: note = Synthetic: Anti-EGFR VL CDR1 AA Seq
QASQDISNYL

SEQ ID NO: 75 - protein, 6 aa, synthetic construct
  REGION 1..6: note = Synthetic: Anti-EGFR VL CDR2 AA Seq
ASNLET

SEQ ID NO: 76 - protein, 9 aa, synthetic construct
  REGION 1..9: note = Synthetic: Anti-EGFR VL CDR3 AA Seq
QHFDHLPLA

SEQ ID NO: 77 - protein, 123 aa, synthetic construct
  REGION 1..123: note = Synthetic: Anti-NRP1 VH AA Seq
EVQLLESGGGLVQPGGSLRLSCAASGFTFSSYAMSWVRQAPGKGLEWVSQISPAGGYTNYADSVKGRFTISRDNSKNTLYLQMNSLRAEDTAVYYCARGELPYFRMSKVMDVWGQGTLVTVSS

SEQ ID NO: 78 - protein, 108 aa, synthetic construct
  REGION 1..108: note = Synthetic: Anti-NRP1 VL AA Seq
EIVLTQSPGTLSLSPGERATLSCRASQFLSSYLAWYQQKPGQAPRLLIYGASARASGIPDRFSGSDGSGTDFTLTISRLEPEDFAVYYCQQYLASPATFGQGTKVEIK

SEQ ID NO: 79 - protein, 8 aa, synthetic construct
  REGION 1..8: note = Synthetic: Anti-NRP1 VH CDR1 AA Seq
FTFSSYAM

SEQ ID NO: 80 - protein, 16 aa, synthetic construct
  REGION 1..16: note = Synthetic: Anti-NRP1 VH CDR2 AA Seq
QISPAGGYTNYADSVK

SEQ ID NO: 81 - protein, 14 aa, synthetic construct
  REGION 1..14: note = Synthetic: Anti-NRP1 VH CDR3 AA Seq
GELPYFRMSKVMDV

SEQ ID NO: 82 - protein, 14 aa, synthetic construct
  REGION 1..14: note = Synthetic: Anti-NRP1 VH CDR3 AA Seq
GELPYHRMSKVMDV

SEQ ID NO: 83 - protein, 14 aa, synthetic construct
  REGION 1..14: note = Synthetic: Anti-NRP1 VH CDR3 AA Seq
GELPYYQMSKVMDV

SEQ ID NO: 84 - protein, 14 aa, synthetic construct
  REGION 1..14: note = Synthetic: Anti-NRP1 VH CDR3 AA Seq
GELPFFRMSQVMDV

SEQ ID NO: 85 - protein, 11 aa, synthetic construct
  REGION 1..11: note = Synthetic: Anti-NRP1 VL CDR1 AA Seq
RASQFLSSYLA

SEQ ID NO: 86 - protein, 11 aa, synthetic construct
  REGION 1..11: note = Synthetic: Anti-NRP1 VL CDR1 AA Seq
RASQYFSSYLA

SEQ ID NO: 87 - protein, 11 aa, synthetic construct
  REGION 1..11: note = Synthetic: Anti-NRP1 VL CDR1 AA Seq
RASQFLSSFLA

SEQ ID NO: 88 - protein, 7 aa, synthetic construct
  REGION 1..7: note = Synthetic: Anti-NRP1 VL CDR2 AA Seq
GASARAS

SEQ ID NO: 89 - protein, 9 aa, synthetic construct
  REGION 1..9: note = Synthetic: Anti-NRP1 VL CDR3 AA Seq
QQYLASPAT